(19) United States
(12) Patent Application Publication
(10) Pub. No.: US 2020/0354441 A1
(43) Pub. Date: Nov. 12, 2020

(54) TREATMENT OF DISORDERS

(71) Applicant: Apellis Pharmaceuticals, Inc., Waltham, MA (US)

(72) Inventor: Cedric Francois, Prospect, KY (US)

(21) Appl. No.: 16/760,390

(22) PCT Filed: Oct. 30, 2018
(86) PCT No.: PCT/US2018/058291

§ 371 Date: Apr. 29, 2020

Related U.S. Application Data
(60) Provisional application No. 62/579,131, filed on Oct. 30, 2017.

(51) Int. Cl. C07K 16/18; G01N 33/50

(52) U.S. Cl. CPC C07K 16/18; G01N 33/502; A61K 2039/505; G01N 2800/7095; G01N 2333/4716

(57) ABSTRACT

In some aspects, the disclosure provides methods of treating a subject in need of treatment for a chronic inflammatory disorder or autoimmune disorder. In some embodiments provided methods comprise administering an iC3b/C3dg/C3d inhibitor to the subject. In some embodiments the methods comprise administering an iC3b/C3dg/C3d inhibitor and/or a complement inhibitor to the subject, so that the subject is exposed to both. For example, in some embodiments, provided methods may comprise administering a complement inhibitor to a subject who has received one or more doses of an iC3b/C3dg/C3d inhibitor, or may comprise administering an iC3b/C3dg/C3d inhibitor to a subject who has received one or more doses of a complement inhibitor. Also provided are methods of identifying candidate agents for treatment of chronic inflammatory or autoimmune disorders, and/or of characterizing agents for use in such treatment.

### CROSS REFERENCE TO RELATED APPLICATIONS

The present application claims priority to U.S. provisional patent application No. 62/579,131, filed Oct. 30, 2017, the entire contents of which are hereby incorporated by reference.

### BACKGROUND

Complement is a system that includes more than 30 secreted and cell-bound proteins and plays a significant role in both innate and adaptive immunity. The proteins of the complement system act in a series of enzymatic cascades through a variety of protein interactions and cleavage events. Complement activation occurs via three main pathways: the antibody-dependent classical pathway, the alternative pathway, and the mannose-binding lectin (MBL) pathway, all of which lead to a common terminal pathway.

Inappropriate or excessive complement activation is an underlying cause or contributing factor to a number of serious diseases and conditions, and considerable effort has been devoted over the past several decades to exploring various complement inhibitors as therapeutic agents.

### SUMMARY

In some aspects, described herein is a method of treating a subject who has a disorder associated with pathologic C3 fragment accumulation on self cells, the method comprising administering an iC3b/C3dg/C3d inhibitor to the subject.

In some aspects, described herein is a method of treating a subject who has a chronic inflammatory disorder or autoimmune disorder, the method comprising administering an iC3b/C3dg/C3d inhibitor to the subject.

In some aspects, described herein is a method of treating a subject who has a chronic inflammatory disorder or autoimmune disorder comprising administering to the subject one or both of (a) an iC3b/C3dg/C3d inhibitor and (b) a complement inhibitor so that the subject is exposed to both the iC3b/C3dg/C3d inhibitor and the complement inhibitor.

In some embodiments the subject has a disorder associated with aberrantly high level of C3 fragment accumulation on self cells.

In some embodiments the iC3b/C3dg/C3d inhibitor inhibits pathologic macrophage/microglial polarization.

In some embodiments the iC3b/C3dg/C3d inhibitor binds to iC3b, C3dg, and/or C3d and inhibits interaction with CR3, CR4, or both.

In some aspects, described herein is a method of characterizing an agent for treatment of a chronic inflammatory disease or autoimmune disease, the method comprising steps of: (a) determining a cell surface level of one or more of iC3b, C3dg, and C3d on mammalian cells when the agent is present; and (b) comparing the determined level with that observed under otherwise comparable conditions absent the agent or when a reference agent of known effect on the cell surface level(s) is present.

In some aspects, described herein is a method of characterizing an agent for treatment of a chronic inflammatory disease or autoimmune disease, the method comprising steps of: (a) determining extent of macrophage or microglia polarization toward a tissue destructive, pro-inflammatory phenotype when the agent is present; and (b) comparing the determined extent with that observed under otherwise comparable conditions absent the agent or when a reference agent of known effect on extent of macrophage or microglia polarization toward the tissue destructive, pro-inflammatory phenotype is present.

All articles, books, patent applications, patents, other publications, websites, and databases mentioned in this application are incorporated herein by reference. In the event of a conflict between the specification and any of the incorporated references the specification (including any amendments thereto) shall control. Unless otherwise indicated, art-accepted meanings of terms and abbreviations are used herein.

### BRIEF DESCRIPTION OF THE DRAWING

FIG. 1 is a schematic diagram showing steps in the pathway of C3 activation and inactivation. (a) shows intact C3, composed of beta and alpha chains linked by a disulfide bond. The alpha chain has an intramolecular disulfide bond. C3 convertases cleave intact C3 to yield C3a and C3b (b). Cleavage of C3b in the fluid phase or on self cell surfaces removes C3f, resulting in the proteolytically inactive iC3b fragment (c). Next, cleavage of iC3b results in C3c and C3dg (d). C3dg remains attached to surfaces and can be cleaved into C3d and C3g (e).

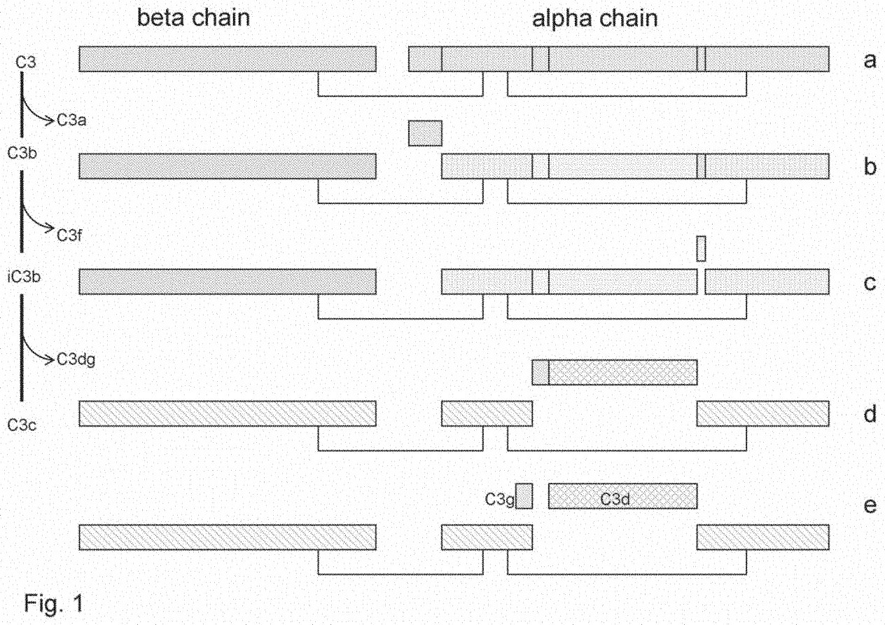

### DETAILED DESCRIPTION OF CERTAIN EMBODIMENTS

### I. Glossary

As used herein, the term “antibody” refers to an immunoglobulin and encompasses full size antibodies and antibody fragments comprising an antigen binding site. Antibodies useful in certain embodiments of the invention may originate from or be derived from a mammal, e.g., a human, non-human primate, rodent (e.g., mouse, rat, rabbit), goat, camelid, or from a bird (e.g., chicken), and may be of any of the various antibody isotypes, e.g., the mammalian isotypes: IgG (e.g., of the IgGl, IgG2, IgG3, or IgG4 subclass), IgM, IgA, IgD, and IgE or isotypes that are not found in mammals, e.g., IgY (found in birds) or IgW (found in sharks). An antibody fragment (Fab) may be, for example, a Fab′, F(ab′)₂, scFv (single-chain variable), single domain antibody (e.g., a VHH), or other fragment that retains or contains an antigen binding site. See, e.g., Allen, T., Nature Reviews Cancer, Vol. 2, 750-765, 2002, and references therein. Antibodies known in the art as diabodies, minibodies, or nanobodies can be used in various embodiments. Bispecific or multispecific antibodies may be used in various embodiments. The heavy and light chain of IgG immunoglobulins (e.g., rodent or human IgGs) contain four framework regions (FR1 through FR4) separated respectively by three complementarity determining regions (CDR1 through CDR3). The CDRs, particularly the CDR3 regions, especially the heavy chain CDR3, are largely responsible for antibody specificity. An antibody may be a chimeric antibody in which, for example, a variable domain of non-human origin, e.g., of rodent (e.g., murine) or non-human primate origin) is fused to a constant domain of human origin, or a “humanized” antibody in which some or all of the complementarity-determining region (CDR) amino acids that constitute an antigen binding site (sometimes along with one or more framework amino acids or regions) are “grafted” from a rodent antibody (e.g., murine antibody) or phage display antibody to a human antibody, thus retaining the specificity of the rodent or phage display antibody. Thus, humanized antibodies may be recombinant proteins in which only the antibody complementarity-determining regions are of non-human origin. It will be appreciated that the alterations to antibody sequence that are involved in the humanization process are generally carried out through techniques at the nucleic acid level, e.g., standard recombinant nucleic acid techniques. In some embodiments only the specificity determining residues (SDRs), the CDR residues that are most crucial in the antibody-ligand interaction, are grafted. The SDRs may be identified, e.g., through use of a database of the three-dimensional structures of the antigen-antibody complexes of known structures or by mutational analysis of the antibody-combining site. In some embodiments an approach is used that involves retention of more CDR residues, namely grafting of so-called “abbreviated” CDRs, the stretches of CDR residues that include all the SDRs. See, e.g., Kashmiri, S V, Methods. 36(1):25-34 (2005), for further discussion of SDR grafting. See, e.g., Almagro J C, Fransson J. Humanization of antibodies. Front Biosci. 13:1619-33 (2008) for review of various methods of obtaining humanized antibodies. It will be understood that “originate from or derived from” refers to the original source of the genetic information specifying an antibody sequence or a portion thereof, which may be different from the species in which an antibody is initially synthesized. For example, “human” domains may be generated in rodents (e.g., mice) whose genome incorporates human immunoglobulin genes or may be generated using phage display. See, e.g., Vaughan, et al, (1998), Nature Biotechnology, 16: 535-539, e.g., for discussion of methods that may be used to generate a fully human antibody. It will be understood that the amino acid sequences of the variable regions of such antibodies are sequences that, while derived from and related to the germline sequences encoding variable domains (V_{H} and/or V_{L} domains) of a particular species (e.g., human), may not naturally exist within that species' antibody germline repertoire in vivo. For example, the human immunoglobulin genes may have been subjected to in vitro mutagenesis (or, when an animal transgenic for human immunoglobulin gene sequences is used, in vivo somatic mutagenesis). An antibody may be polyclonal or monoclonal, though for purposes of the present invention monoclonal antibodies are generally preferred as therapeutic agents. Antibodies may be glycosylated or non-glycosylated. Methods for generating antibodies that specifically bind to virtually any molecule of interest are known in the art. For example, monoclonal or polyclonal antibodies can be purified from natural sources, e.g., from blood or ascites fluid of an animal that produces the antibody (e.g., following immunization with the molecule or an antigenic fragment thereof) or can be produced recombinantly, in cell culture and, e.g., purified from culture medium. Affinity purification may be used, e.g., protein A/G affinity purification and/or affinity purification using the antigen as an affinity reagent. Suitable antibodies can be identified using phage display and related techniques. See, e.g., Kaser, M. and Howard, G., “Making and Using Antibodies: A Practical Handbook” and Sidhu, S., “Phage Display in Biotechnology and Drug Discovery”, CRC Press, Taylor and Francis Group, 2005, for further information. Methods for generating antibody fragments are well known. For example, F(ab′)₂ fragments can be generated, for example, through the use of an Immunopure F(ab′)₂ Preparation Kit (Pierce) in which the antibodies are digested using immobilized pepsin and purified over an immobilized Protein A column. The digestion conditions (such as temperature and duration) may be optimized by one of ordinary skill in the art to obtain a good yield of F(ab′)₂. The yield of F(ab′)₂ resulting from the digestion can be monitored by standard protein gel electrophoresis. F(ab′) can be obtained by papain digestion of antibodies, or by reducing the S—S bond in the F(ab′)₂. As used herein, a “single-chain Fv” or “scFv” antibody fragment comprises the V_{H} and V_{L} domains of an antibody, wherein these domains are present in a single polypeptide chain. Typically, a scFv antibody further comprises a polypeptide linker between the V_{H} and V_{L} domains, although other linkers could be used to connect the domains in certain embodiments.

In some embodiments an antibody may be selected or designed to have any of a variety of desired features and/or to lack any of a variety of non-desired feratures. For example, in some embodiments an antibody lacks or substantially lacks one or more effector functions. Antibody effector functions include antibody-dependent cell-mediated cytotoxicity (ADCC), antibody-dependent phagocytosis (ADP), complement activation, and complement-dependent cytotoxicity (CDC). Antibody effector functions are mediated via the Fc domain of the antibody. ADCC and ADP depend strongly on interactions between the Fc domain and certain Fcγ receptors (e.g., (FcγRI, FcγRIIa, and FcγRIIIa) on natural killer cells, macrophages, and other immune cells. CDC depends on the interaction of Fc domains with C1q. As known in the art, effector functions vary between different antibody classes and subclasses. For example, the IgG3 and IgG1 subclasses have the highest and second highest complement activating activities among the IgG subclasses, while IgG4 lacks complement activating activity. Residues involved in mediating the effector functions have been identified (e.g., in the CHI, hinge, CH2, and/or CH3 portion of the heavy chain), and antibodies can be designed or engineered to reduce effector activity using known methods, such as by introducing alterations into such residues (e.g., substituting alanine for a residue identified as involved in mediating the effector activity of interest) or utilizing CH domains or hinge regions from antibody subclasses that lack the effector activity. In some embodiments, the antibody comprises a CH2 domain that has reduced ability to bind C1q as compared with a human IgG1 CH2 domain. The antibody may, for example, contain a CH2 domain having alterations in one or more of residues relative to a naturally occurring CH2 domain. In some embodiments, the antibody comprises a human IgG4 heavy chain constant region or a variant thereof. In some embodiments, the antibody contains CHI, CH2, and/or CH3 domains from human IgG4. For example, in some embodiments the antibody contains at least CH2 and CH3 from human IgG4. In some embodiments, the antibody does not contain CHI, CH2, and/or CH3 domains from a human IgG1. For example, in some embodiments the antibody does not contain CH2 and CH3 from a human IgG1. In some embodiments the antibody comprises a human IgG2 heavy chain constant region for a variant thereof. In some embodiments the antibody comprises at least CH2 and CH3 from a human IgG2. Examples of antibodies (e.g., variants of IgG2) having reduced or undetectable levels of effector functions are disclosed in US Patent App. Pub. Nos. US20070148167 and 20110212087. In some embodiments, an Fc domain from such antibodies, or substitutions in the Fc domain described in the afore-mentioned publications, may be utilized in the antibodies of the present disclosure. Antibody effector functions may be measured as described in the afore-mentioned US publications or in Tada, M., et al., PLoS One. 9(4):e95787, 2014 (for ADCC) using target cells that express the target of the antibody whose effector function is being assessed. In some embodiments an effector function (e.g., ADCC) may be measured based on binding to FcγRI, FcγRIIa, and FcγRIIIa, e.g., as described in Velayudhan, J BioDrugs. 2016; 30: 339-351.

The terms “approximately” or “about” in reference to a number generally include numbers that fall within ±10%, in some embodiments ±5%, in some embodiments ±1%, in some embodiments ±0.5% of the number unless otherwise stated or otherwise evident from the context (except where such number would impermissibly exceed ±100% of a possible value).

As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied. “Complement activation capacity” refers to the level of complement activation that would result from exposure to a stimulus that causes maximum complement activation. Typically, complement activation capacity is assessed using a sample obtained from a subject (e.g., a blood, plasma, serum, or other fluid sample, which may be diluted appropriately), which sample may be exposed in vitro to a complement activating stimulus. A heat-inactivated sample can be used as a control. It will be understood that the stimulus need not be sufficient to cause maximum complement activation in order to provide a measurement of complement activation capacity. For example, the extent to which complement activation occurs within a defined time period can provide an indication of complement activation capacity.

A “complement component” or “complement protein” is a protein that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, and properdin (also called factor P). Components of the lectin pathway include, e.g., MBL2, MASP-1, MASP-2, and MASP-3. Complement components also include cell-bound receptors for soluble or cell-bound complement components, wherein such receptor mediates one or more biological activities of such soluble complement component following binding of the soluble complement component. Such receptors include, e.g., C5a receptor (C5aR), C5a receptor 2 (C5aR2, often referred to as C5L2), C3a receptor (C3aR), Complement Receptor 1 (CR1), Complement Receptor 2 (CR2), Complement Receptor 3 (CR3, also known as CD45), Complement Receptor 4 (CR4), and Complement receptor of the immunoglobulin family (CRIg) It will be appreciated that the term “complement component” is not intended to include those molecules and molecular structures that serve as “triggers” for complement activation, e.g., antigen-antibody complexes, foreign structures found on microbial or articifial surfaces, etc.

A “complement regulatory protein” is a protein involved in regulating complement activity. A complement regulatory protein may down-regulate complement activity by, e.g., inhibiting complement activation or by inactivating or accelerating decay of one or more activated complement proteins. Examples of complement regulatory proteins include C1 inhibitor, C4 binding protein, clusterin, vitronectin, complement factor H (CFH, sometimes referred to as FH), complement factor I (CFI, sometimes referred to as FI or IF), and the cell-bound proteins CD46, CD55, CD59, CR1, CR2, and CR3. In certain embodiments C-reactive protein is considered a complement regulatory protein. In certain embodiments C-reactive protein is not considered a complement regulatory protein.

A “complement control protein” is a complement regulatory protein comprising multiple SCR modules as described below. Examples include CFH, CD46, CD55, CR1, and CR2.

A “complement-like protein” is a protein that has significant sequence identity to a complement protein or a complement control protein over at least 20% of its length and/or specifically competes with the complement protein or complement control protein for binding to its target, e.g., has an affinity at least 10% as great. The genes encoding such proteins may be found in close proximity to genes encoding the complement protein or complement control protein having a similar sequence. For example, the CFH gene cluster contains numerous CFH-like genes (e.g., CFHR1, CFHR2, CFHR3, CFHR4, and CFHR5).

“Complement-related protein” refers collectively to complement components, complement regulatory proteins, and complement-like proteins; however, wherever the disclosure refers to complement-related proteins in general, it is understood that the invention encompasses embodiments that relate specifically to complement components, complement regulatory proteins, complement-like proteins, and any combination or subset thereof. Any one or more ccomplement-related proteins, whether or not expressly mentioned herein, may be excluded from any one or more embodiments.

As used herein, the term “complement-mediated disorder” is a disorder in which complement activation (e.g., excessive or inappropriate complement activation) is involved, e.g., as a contributing and/or at least partially causative factor. In some embodiments a complement-mediated disorder is one in which one or more complement system biomarkers, e.g., one or more genetic markers, is known to be associated with likelihood of developing or having the disorder, such that results of an assay of such complement system biomarker(s) are indicative of the likelihood that a subject who does not have the disorder will develop the disorder or of the likelihood that a subject has the disorder.

As used herein, the term “immune system cell” or “immune cell” refers to any of a variety of cells that play a role in the immune response. Immune system cells include lymphocytes (T cells, B cells, natural killer (NK) cells); dendritic cells, monocytes, macrophages, eosinophils, mast cells, basophils, and neutrophils. T cells encompass a number of different functional classes that play different roles in the immune response. Different functional classes may be distinguished based on cell surface markers and other properties. Most T cells express an alpha beta (ap) T cell receptor (TCR) through which the cell is able to recognize a specific antigen in the context of an appropriate major histocompatibility complex (MHC) molecule, though a minor subset expresses the γδ TCR. Cytotoxic T cells (CTLs) are typically positive for the cell surface marker CD8, which serves as a co-receptor for the TCR in recognition of MHC Class I molecules on the surface of target cells during antigen-specific T cell activation and/or responses. CTLs and NK cells play important roles by eliminating infected host cells and tumor cells through a variety of mechanisms including the release of cytotoxic substances. Helper T cells are typically positive for the cell surface marker CD4, which serves as a co-receptor for the TCR in recognition of MHC Class II molecules on the surface of APCs during antigen-specific T cell activation. Helper T cells promote the activity of other immune system cells (i.e., provide “help”) by, among other things, releasing cytokines that have a variety of effects such as enhancing survival, proliferation, and/or differentiation. Natural killer cells have the ability to recognize and kill (e.g., by causing lysis or apoptosis) cancerous, stressed, or infected cells without requiring antigen-specific activation by presentation of antigen in the context of MHC. Instead, their activation is regulated by a balance of the activity of activating receptors and inhibitory receptors and cytokines. NK cells typically lack cell surface receptors that are highly specific for a particular antigen and are able to react rapidly without prior exposure to the antigen. As used herein, “effector cells” refers to the activated immune system cells that defend the body in an immune response. Effector T cells include cytotoxic T cells and helper T cells, which carry out cell-mediated responses. Effector B cells are called plasma cells and secrete antibodies. Effector cells also include effector NK cells. Regulatory T cells (Tregs) are a subset of CD4⁺ T cells whose normal roles are to suppress immune responses and maintain self-tolerance. The transcription factors FoxP3 and STAT5 play important roles in their development. Tregs are often CD4⁺CD25⁺ FoxP3⁺ and may be identified based on a cell surface marker expression pattern of CD4⁺CD25⁺CD127^{lo}. Tregs may also be characterized by expression of CTLA4 and GITR. Tregs may suppress the activity of other immune system cell subsets by a variety of mechanisms such as secretion of immunosuppressive cytokines and via cell-cell contact. They may inhibit immune responses at multiple steps, e.g., at the induction of activation (e.g., by inhibiting the ability of APCs to stimulate T cells) and during effector phases. Deficency or impaired functional activity of Tregs has been associated with autoimmune disorder. Where it is intended herein to refer to a T cell that is a Treg, the T cell will be identified as such. Thus, unless expressly indicated a T cell, as used herein, is not a Treg cell. An antigen-presenting cell (APC) is a cell that can process and display antigens in association with major histocompatibility complex (MHC) molecules on its surface. T cells may recognize these complexes using their T cell receptors (TCRs). APCs may also display other molecules (costimulatory proteins) that are required for activating naïve T cells. APCs that express MHC class II molecules include dendritic cells, macrophages, and B cells and may be referred to as professional APCs. Dendritic cells (DCs) are white blood cells that occur in most tissues of the body, particularly epithelial tissues. DCs serve as a link between peripheral tissues and lymphoid organs. Immature DCs sample the surrounding environment and take up antigenic substances such as pathogen components or tumor antigens. They undergo maturation and migrate to lymph nodes or spleen, where they display fragments of processed antigens at their cell surface using MHC Class II (MHCII) complexes. As part of the maturation process, DCs upregulate cell-surface molecules that act as co-stimulators in T cell activation, such as CD80 (B7-1), CD86 (B7-2), and/or CD40. DCs activate helper T cells by presenting them with antigens in the context of MHCII complexes, together with non-antigen specific co-stimulators. DCs and various other APCs have the capacity to activate cytotoxic T cells and B cells through presentation of MHC Class I (MHCI)-peptide complexes (cross-presentation) and costimulators.

“Linked”, as used herein with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In some embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linking moiety” or “linking portion”, the linkage between the two linked moieties is indirect, and typically each of the linked moieties is covalently bonded to the linking moiety. Two moieties may be linked using a “linker”. A linker can be any suitable moiety that reacts with the entities to be linked within a reasonable period of time, under conditions consistent with stability of the entities (portions of which may be protected as appropriate, depending upon the conditions), and in sufficient amount, to produce a reasonable yield. Typically the linker will contain at least two functional groups, one of which reacts with a first entity and the other of which reacts with a second entity. It will be appreciated that after the linker has reacted with the entities to be linked, the term “linker” may refer to the part of the resulting structure that originated from the linker, or at least the portion that does not include the reacted functional groups. A linking moiety may comprise a portion that does not participate in a bond with the entities being linked, and whose main purpose may be to spatially separate the entities from each other. Such portion may be referred to as a “spacer”.

“Polypeptide”, as used herein, refers to a polymer of amino acids, optionally including one or more amino acid analogs. A protein is a molecule composed of one or more polypeptides. A peptide is a relatively short polypeptide, typically between about 2 and 60 amino acids in length, e.g., between 8 and 40 amino acids in length. The terms “protein”, “polypeptide”, and “peptide” may be used interchangeably. Polypeptides used herein may contain amino acids such as those that are naturally found in proteins, amino acids that are not naturally found in proteins, and/or amino acid analogs that are not amino acids. As used herein, an “analog” of an amino acid may be a different amino acid that structurally resembles the amino acid or a compound other than an amino acid that structurally resembles the amino acid. A large number of art-recognized analogs of the 20 amino acids commonly found in proteins (the “standard” amino acids) are known. One or more of the amino acids in a polypeptide may be modified, for example, by the addition of a chemical entity such as a carbohydrate group, a phosphate group, a farnesyl group, an isofamesyl group, a fatty acid group, a linker for conjugation, functionalization, or other modification, etc. Certain non-limiting suitable analogs and modifications are described in WO2004026328 and/or below. The polypeptide may be acetylated, e.g., at the N-terminus and/or amidated, e.g., at the C-terminus.

In general, polypeptides may be obtained or produced using any suitable method known in the art. For example, polypeptides may be isolated from natural sources, produced in vitro or in vivo using recombinant DNA technology in suitable expression systems (e.g., by recombinant host cells or transgenic non-human animals or plants), synthesized through chemical means such as solid phase peptide synthesis and/or using methods involving chemical ligation of synthesized peptides (see, e.g., Kent, S., J Pept Sci., 9(9):574-93, 2003 and U.S. Pub. No. 20040115774), or a combination of these. One of ordinary skill in the art would readily select appropriate method(s). A polypeptide may comprise a tag, e.g., an epitope tag, which tag may facilitate purification and/or detection of the polypeptide. Exemplary tags include, e.g., 6×His, HA, Myc, SNUT, FLAG, TAP, V5, TEV, Thrombin, PreScission, etc. In some embodiments, a tag is cleavable, e.g., the tag comprises a recognition site for cleavage by a protease, or is separated from a portion complement inhibiting portion of the polypeptide by a linking portion that comprises a recognition site for cleavage by a protease. For example, a TEV protease cleavage site can be used.

As used herein, the term “purified” refers to agents that have been separated from most of the components with which they are associated in nature or when originally generated or with which they were associated prior to purification. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate. Any of the agents or substances described herein may be purified.

“Recombinant host cells”, “host cells”, and other such terms, denote prokaryotic or eukaryotic cells or cell lines that contain an exogenous nucleic acid (typically DNA) such as an expression vector comprising a nucleic acid that encodes a polypeptide of interest. It will be understood that such terms include the descendants of the original cell(s) into which the vector or other nucleic acid has been introduced. Appropriate host cells include any of those routinely used in the art for expressing polynucleotides (e.g., for purposes of producing polypeptide(s) encoded by such polynucleotides) including, for example, prokaryotes, such as E. coli; and eukaryotes, including for example, fungi, such as yeast (e.g., Pichia pastoris); insect cells (e.g., Sf9), plant cells, and animal cells, e.g., mammalian cells such as CHO, RI.1, B-W, L-M, African Green Monkey Kidney cells (e.g. COS-1, COS-7, BSC-1, BSC-40 and BMT-10) and cultured human cells. The exogenous nucleic acid may be stably maintained as an episome such as a plasmid or may at least in part be integrated into the host cell's genome, optionally after being copied or reverse transcribed. Terms such as “host cells”, etc., are also used to refer to cells or cell lines that can be used as recipients for an exogenous nucleic acid, prior to introduction of the nucleic acid. A “recombinant polynucleotide” is a polynucleotide that contains nucleic acid sequences that are not found joined directly to one another in nature. For example, the nucleic acid sequences may occur in different genes or different species or one or more of the sequence(s) may be a variant of a naturally occurring sequence or may at least in part be an artificial sequence that is not homologous to a naturally occurring sequence. A “recombinant polypeptide” is a polypeptide that is produced by transcription and translation of an exogenous nucleic acid by a recombinant host cell or by a cell-free in vitro expression system and/or that contains amino acid sequences that are not found joined directly to one another in nature. In the latter case, the recombinant polypeptide may be referred to as a “chimeric polypeptide”. The amino acid sequences in a chimeric polypeptide may, for example, occur in different genes or in different species or one or more of the sequence(s) may be a variant of a naturally occurring sequence or may at least in part be an artificial sequence that is not homologous to a naturally occurring sequence. It will be understood that a chimeric polypeptide may comprise two or more polypeptides. For example, first and second polypeptides A and B of a chimeric polypeptide may be directly linked (A-B or B-A) or may be separated by a third polypeptide portion C (A-C-B or B-C-A). In some embodiments, portion C represents a polypeptide linker which may, for example, comprise multiple glycine and/or serine residues. In some embodiments, two or more polypeptides may be linked by non-polypeptide linker(s).

“Reactive functional groups” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds, N-hydroxysuccinimide esters, maleimides, sulfhydryls, and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989, and Hermanson, G., Bioconjugate Techniques, 2^{nd} ed., Academic Press, San Diego, 2008).

As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

“Specific binding” generally refers to a physical association between a target molecule (e.g., a polypeptide) or molecular complex and a binding molecule such as an antibody or ligand. The association is typically dependent upon the presence of a particular structural feature of the target such as an antigenic determinant, epitope, binding pocket or cleft, recognized by the binding molecule. For example, if an antibody is specific for epitope A, the presence of a polypeptide containing epitope A or the presence of free unlabeled A in a reaction containing both free labeled A and the binding molecule that binds thereto, will reduce the amount of labeled A that binds to the binding molecule. It is to be understood that specificity need not be absolute but generally refers to the context in which the binding occurs. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies or ligands having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target versus the affinity of the binding molecule for other targets, e.g., competitors. If a binding molecule exhibits a high affinity for a target molecule that it is desired to detect and low affinity for nontarget molecules, the antibody will likely be an acceptable reagent. Once the specificity of a binding molecule is established in one or more contexts, it may be employed in other, preferably similar, contexts without necessarily re-evaluating its specificity. In some embodiments, the affinity (as measured by the equilibrium dissociation constant, Kd) of two molecules (or between a molecule and a complex), e.g., two molecules that exhibit specific binding, is 10⁻³ M or less, e.g., 10⁻⁴ M or less, e.g., 10⁻⁵ M or less, e.g., 10⁻⁶M or less, 10⁻⁷M or less, 10⁻⁸M or less, 10⁻⁹ M or less, 10⁻¹⁰ M or less, 10⁻¹¹ M or less, 10⁻¹² M or less, e.g., between 10-13 M and 10⁻³ M (or within any range having any two of the afore-mentioned values as endpoints) under the conditions tested, e.g., under physiological conditions (e.g., conditions such as salt concentration, pH, and/or temperature, etc., that reasonably approximate corresponding conditions in vivo), or other conditions of the assay. Binding affinity can be measured using any of a variety of methods known in the art. For example, assays based on isothermal titration calorimetry or surface plasmon resonance (e.g., Biacore® assays) can be used in certain embodiments. Additionally or alternately, ELISA assays (such as competitive ELISA assays), radioligand assays, fluorescence polarization,

As used herein, a “self cell” refers to an individual's own cells, as contrasted with cells of a different individual or cells of a pathogen.

A “specific binding agent” is an agent that exhibits specific binding to a target.

A “subject” is typically a human, a non-human primate, or another mammal such as a cow, horse, dog, cat, rodent (e.g., mouse or rat), or rabbit. It will be appreciated that, at least in embodiments wherein a complement inhibitor is administered, the subject should express at least one complement component that can be inhibited by the particular complement inhibitor used. For example, a complement inhibitor specific for primate complement would typically be administered to a human or non-human primate or an animal model that has been genetically engineered to express human complement component(s). In some embodiments the subject is male. In some embodiments the subject is female. In some embodiments, a human subject is at least 12 years of age. In some embodiments a subject is an adult, e.g., a human at least 18 years of age, e.g., between 18 and 100 years of age. The term “subject in need of treatment for cancer”, means a subject who has cancer, e.g., a subject who has been diagnosed as suffering from a cancer, and includes subjects in whom the presence of cancer is detectable as well as subjects who receive adjuvant therapy, for example after surgical removal of a cancer, in an effort to eradicate any residual cancer cells. A subject may sometimes be referred to herein as a “patient” or “individual”. A subject in need of treatment for cancer may be referred to as a “cancer patient”. Any method described herein may be expressly limited to human subjects. Where methods relate to particular biomarkers known to exist in humans and not known to exist in non-human animals, it will be understood that the subject or sample in which such biomarker is assayed is typically a human subject or sample obtained from a human subject.

“Treating”, as used herein in regard to treating a subject, refers to providing treatment, i.e, providing any type of medical or surgical management of a subject. The treatment can be provided in order to reverse, alleviate, inhibit the progression of, prevent or reduce the likelihood of a disease, or in order to reverse, alleviate, inhibit or prevent the progression of, prevent or reduce the likelihood of one or more symptoms or manifestations of a disease. “Prevent” refers to causing a disease or symptom or manifestation of a disease not to occur for at least a period of time in at least some individuals, e.g., individuals at risk of developing the disease, symptom, or manifestation. Treating may include administering an agent, carrying out a procedure (e.g., a surgical procedure), or both, for the purposes of obtaining an effect. Treating can include administering a compound or composition to the subject following the development of one or more symptoms or manifestations indicative of a disease, e.g., in order to reverse, alleviate, reduce the severity of, and/or inhibit or prevent the progression of the disease and/or to reverse, alleviate, reduce the severity of, and/or inhibit or one or more symptoms or manifestations of the disease. A compound or composition can be administered to a subject who has developed a disease, or is at increased risk of developing the disease relative to a member of the general population, optionally a member who is matched with the subject in terms of age, sex, and/or other demographic variable(s). It will be understood that a compound, composition, or intervention useful to treat a subject may be referred to as a “treatment” or “therapy”.

A “variant” of a particular polypeptide or polynucleotide has one or more alterations (e.g., additions, substitutions, and/or deletions, which may be referred to collectively as “mutations”) with respect to the polypeptide or nucleic acid, which may be referred to as the “original polypeptide” or “original polynucleotide”, respectively. Thus a variant can be shorter or longer than the polypeptide or polynucleotide of which it is a variant. The terms “variant” encompasses “fragments”. A “fragment” is a continuous portion of a polypeptide that is shorter than the original polypeptide. In certain embodiments of the invention a variant polypeptide has significant sequence identity to the original polypeptide over a continuous portion of the variant that comprises at least 50%, preferably at least 60%, at least 70%, at least 80%, at least 90%, at least 95%, or more, of the length of the variant or the length of the polypeptide, (whichever is shorter). In certain embodiments of the invention a variant polypeptide has substantial sequence identity to the original polypeptide over a continuous portion of the variant that comprises at least 50%, preferably at least 60%, at least 70%, at least 80%, at least 90%, at least 95%, or more, of the length of the variant or the length of the polypeptide, (whichever is shorter). In a non-limiting embodiment a variant has at least 80% identity to the original sequence over a continuous portion of the variant that comprises between 90% and 100% of the variant, e.g., over 100% of the length of the variant or the length of the polypeptide, (whichever is shorter). In another non-limiting embodiment a variant has at least 80% identity to the original sequence over a continuous portion of the variant that comprises between 90% and 100% of the variant, e.g., over 100% of the length of the variant or the length of the polypeptide, (whichever is shorter). In specific embodiments the sequence of a variant polypeptide has N amino acid differences with respect to an original sequence, wherein N is any integer between 1 and 10. In other specific embodiments the sequence of a variant polypeptide has N amino acid differences with respect to an original sequence, wherein N is any integer between 1 and 20. An amino acid “difference” refers to a substitution, insertion, or deletion of an amino acid.

In certain embodiments a fragment or variant possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue):W522-5, Jul. 1, 2004). In many embodiments one, more than one, or all biological functions or activities of a variant or fragment is substantially similar to that of the corresponding biological function or activity of the original molecule. In certain embodiments the activity of a variant or fragment may be at least 20%, at least 50%, at least 60%, at least 70%, at least 80%, or at least 90% of the activity of the original molecule, up to approximately 100%, approximately 125%, or approximately 150% of the activity of the original molecule. In certain embodiments an activity of a variant or fragment is such that the amount or concentration of the variant needed to produce an effect is within 0.5 to 5-fold of the amount or concentration of the original molecule needed to produce that effect. The disclosure contemplates use of variants of any of the complement inhibiting polypeptides disclosed herein, wherein the variant inhibits complement sufficiently to be useful in a method described herein. In some embodiments, a variant lacks or has a substantially reduction in a property that may be undesired such as immunogenicity.

As used herein, “alkyl” refers to a saturated straight, branched, or cyclic hydrocarbon having from about 1 to about 22 carbon atoms (and all combinations and subcombinations of ranges and specific numbers of carbon atoms therein), with from about 1 to about 12, or about 1 to about 7 carbon atoms being preferred in certain embodiments of the invention. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

As used herein, “halo” refers to F, Cl, Br or I.

As used herein, “alkanoyl” refers to an optionally substituted straight or branched aliphatic acyclic residue having about 1 to 10 carbon atoms (and all combinations and subcombinations of ranges and specific number of carbon atoms) therein, e.g., from about 1 to 7 carbon atoms which, as will be appreciated, is attached to a terminal C═O group with a single bond (and may also be referred to as an “acyl group”). Alkanoyl groups include, but are not limited to, formyl, acetyl, propionyl, butyryl, isobutyryl, pentanoyl, isopentanoyl, 2-methyl-butyryl, 2,2-dimethoxypropionyl, hexanoyl, heptanoyl, octanoyl, and the like, and for purposes of the present invention a formyl group is considered an alkanoyl group. “Lower alkanoyl” refers to an optionally substituted straight or branched aliphatic acyclic residue having about 1 to about 5 carbon atoms (and all combinations and subcombinations of ranges and specific number of carbon atoms). Such groups include, but are not limited to, formyl, acetyl, propionyl, butyryl, isobutyryl, pentanoyl, isopentanoyl, etc.

As used herein, “aryl” refers to an optionally substituted, mono- or bicyclic aromatic ring system having from about 5 to about 14 carbon atoms (and all combinations and subcombinations of ranges and specific numbers of carbon atoms therein), with from about 6 to about 10 carbons being preferred. Non-limiting examples include, for example, phenyl and naphthyl.

As used herein, “aralkyl” refers to alkyl radicals bearing an aryl substituent and having from about 6 to about 22 carbon atoms (and all combinations and subcombinations of ranges and specific numbers of carbon atoms therein), with from about 6 to about 12 carbon atoms being preferred in certain embodiments. Aralkyl groups can be optionally substituted. Non-limiting examples include, for example, benzyl, naphthylmethyl, diphenylmethyl, triphenylmethyl, phenylethyl, and diphenylethyl.

As used herein, the terms “alkoxy” and “alkoxyl” refer to an optionally substituted alkyl-O— group wherein alkyl is as previously defined. Exemplary alkoxy and alkoxyl groups include methoxy, ethoxy, n-propoxy, i-propoxy, n-butoxy, and heptoxy.

As used herein, “carboxy” refers to a —C(═O)OH group.

As used herein, “alkoxycarbonyl” refers to a —C(═O)O-alkyl group, where alkyl is as previously defined.

As used herein, “aroyl” refers to a —C(═O)-aryl group, wherein aryl is as previously defined. Exemplary aroyl groups include benzoyl and naphthoyl.

The term “cyclic ring system” refers to an aromatic or non-aromatic, partially unsaturated or fully saturated, 3- to 10-membered ring system, which includes single rings of 3 to 8 atoms in size and bi- and tri-cyclic ring systems which may include aromatic 5- or 6-membered aryl or aromatic heterocyclic groups fused to a non-aromatic ring. These heterocyclic rings include those having from 1 to 3 heteroatoms independently selected from the group consisting of oxygen, sulfur, and nitrogen. In certain embodiments, the term heterocyclic refers to a non-aromatic 5-, 6-, or 7-membered ring or a polycyclic group wherein at least one ring atom is a heteroatom selected from the group consisting of O, S, and N, including, but not limited to, a bi- or tri-cyclic group, comprising fused six-membered rings having between one and three heteroatoms independently selected from the group consisting of the oxygen, sulfur, and nitrogen. In some embodiments, “cyclic ring system” refers to a cycloalkyl group which, as used herein, refers to groups having 3 to 10, e.g., 4 to 7 carbon atoms. Cycloalkyls include, but are not limited to cyclopropyl, cyclobutyl, cyclopentyl, cyclohexyl, cycloheptyl and the like, which, is optionally substituted. In some embodiments, “cyclic ring system” refers to a cycloalkenyl or cycloalkynyl moiety, which is optionally substituted.

Typically, substituted chemical moieties include one or more substituents that replace hydrogen. Exemplary substituents include, for example, halo, alkyl, cycloalkyl, aralkyl, aryl, sulfhydryl, hydroxyl (—OH), alkoxyl, cyano (—CN), carboxyl (—COOH), —C(═O)O-alkyl, aminocarbonyl (—C(═O)NH₂), —N-substituted aminocarbonyl (—C(═O)NHR″), CF₃, CF₂CF₃, and the like. In relation to the aforementioned substituents, each moiety R″ can be, independently, any of H, alkyl, cycloalkyl, aryl, or aralkyl, for example.

As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

As used herein, an “aromatic amino acid” is an amino acid that comprises at least one aromatic ring, e.g., it comprises an aryl group.

As used herein, an “aromatic amino acid analog” is an amino acid analog that comprises at least one aromatic ring, e.g., it comprises an aryl group.

### II. Complement System

In order to facilitate understanding of aspects of the present disclosure, and without intending to limit the invention in any way, this section provides an overview of complement and its pathways of activation. Further details are found, e.g., in Paul, W. E., Fundamental Immunology, Lippincott Williams & Wilkins; 7 ed., 2012, and other standard textbooks. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form the classical pathway C3 convertase, which cleaves C3 to form C3a and C3b. Binding of C3b to C3 convertase produces the classical pathway C5 convertase (C4b2b3b complex). C3a, C4a, and C5a are anaphylotoxins and, among other things, mediate multiple reactions in the acute inflammatory response. C3a and C5a are also chemotactic factors that attract immune system cells such as neutrophils.

The alternative pathway is initiated by hydrolysis of a labile thioester bond in C3, which occurs spontaneously at a low level and converts C3 to a bioactive form (C3(H2O)) in the fluid phase, a process sometimes referred to as “tickover”. The rate of hydrolysis of C3 to C3(H₂O) can be increased by interactions between C3 and various biological and artificial surfaces. Upon hydrolysis, a structural change in the thioester domain (TED) of C3 exposes a binding site for factor B, which is then cleaved by factor D, generating a fluid phase C3 convertase (C3(H₂O)Bb) that activates complement by cleaving C3 into C3a and C3b. C3b binds to targets such as microbial cell surfaces and forms a complex with factor B, which is later cleaved by factor D, resulting in a surface-bound C3 convertase (C3bBb, the alternative pathway C3 convertase), which can be stabilized by binding of factor P (properdin). Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the cascade. Furthermore, binding of another molecule of C3b to the C3 convertase gives rise to a C5 convertase.

The lectin complement pathway is initiated by binding of carbohydrate-binding proteins mannose-binding lectin (MBL), CL-I land the ficolins (H-ficolin, M-ficolin, or L-ficolin) and MBL-associated serine protease (MASP) to certain carbohydrates, e.g., mannose, found on pathogen surfaces. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. Multimers of MBL form a complex with MASP-1 (mannose-binding lectin-associated serine protease), MASP-2, and MASP-3, which are protease zymogens. When the carbohydrate-recognising heads of MBL bind to specifically arranged mannose residues, MASP-1 and MASP-2 are activated and cleave complement components C4 and C2 into C4a, C4b, C2a, and C2b, leading to formation of a C3 convertase (C4bC2a), which is also the classical pathway C3 convertase. Binding of C3b to this C3 convertase produces a C5 convertase, as in the classical pathway.

The C5 convertases generated via any of these pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Sub-lytic amounts of MAC on the membrane of cells may have a variety of deleterious consequences.

Complement activity is regulated by a variety of cell bound proteins and plasma proteins that serve to limit complement activation on, and complement-mediated damage to, mammalian cells. Complement factor I (CFI) is a plasma protein that regulates complement activation by cleaving cell-bound or fluid phase C3b and C4b. Complement control proteins (CCPs), also known as regulators of complement activation (RCA) proteins (U.S. Pat. No. 6,897,290) are characterized by the presence of multiple (typically 4-56) homologous motifs known as short consensus repeats (SCR), complement control protein (CCP) modules, or SUSHI domains, which are about 50-70 amino acids in length and contain a conserved motif including four disulfide-bonded cysteines (two disulfide bonds), proline, tryptophan, and many hydrophobic residues. The CCP family includes complement receptor type 1 (CR1; C3b:C4b receptor), complement receptor type 2 (CR2), membrane cofactor protein (MCP; CD46), decay-accelerating factor (DAF), complement factor H (CFH), and C4b-binding protein (C4 bp). CR1, CR2, MCP, and DAF are cell surface proteins, while CFH and C4 bp are plasma proteins. CCPs differ with respect to ligand specificity and mechanism(s) of complement inhibition. They may accelerate the normal decay of convertases and/or function as cofactors for factor I, to enzymatically cleave C3b and/or C4b into smaller fragments. For example, CFH regulates complement activation on self cells and surfaces by possessing both cofactor activity for the CFI-mediated cleavage of C3b and decay accelerating activity against the alternative pathway C3 convertase. Decay-accelerating factor accelerates decay of the alternative pathway C3 convertase. CD59 is a membrane-bound complement regulatory protein that is structurally unrelated to the CCPs. It can prevent C9 from polymerizing and thus inhibits formation of the MAC.

C3b bound to mammalian cell surfaces is rapidly converted to iC3b by the action of CFI and its cofactors (CFH, MCP, CR1). iC3b cannot bind factor B and therefore cannot form C3 convertases. Cleavage of iC3b by CFI (with the assistance of CR1 as a cofactor) generates C3dg, which remains bound to the cell, and C3c, which is released in the fluid phase. C3dg is cleaved by tissue proteases to form C3d, which remains bound to the cell, and C3g, which is released in the fluid phase.

### III. Methods and Compositions Relating to iC3b/C3dg/C3d Inhibition

In some aspects, described herein are methods and compositions useful for treatment of disorders associated with pathologic C3 fragment deposition on self cells. In some aspects, compositions and methods described herein inhibit pathologic macrophage polarization. Macrophages are a type of immune cell that play important roles both in innate and adaptive immunity. They can have a range of phenotypes with associated properties. Macrophages are found in essentially all tissues and can take various forms (with various names) throughout the body. Resident macrophages are macrophages that are normally present in a tissue, as contrasted with macrophages that enter the tissue or develop from monocytes that enter the tissue under pathologic conditions, e.g., in the setting of inflammation or tissue injury. Microglia, which are the primary resident immune cell in the central nervous system, including the retina, are considered a type of macrophage, although they have a number of distinct activities and characteristics. Wherever the present disclosure refers to macrophages, it should be understood to encompass a specific reference to microglia unless otherwise stated or unless the context clearly dictates otherwise. The primary function of macrophages in innate immunity is to carry out phagocytosis, in which they engulf and digest microbes, foreign substances, and cellular debris, as well as dead, dying, or otherwise unhealthy or abnormal cells. Macrophages recognize a variety of molecules that mark cells or structures bearing them as targets for phagocytosis, a process termed “opsonization”. Among these are the C3 degradation products iC3b, C3dg, and C3d, which become covalently attached to surfaces as a result of complement activation and promote phagocytosis. In adaptive immunity, macrophage functions include recruiting other immune cells and presenting antigens to T cells.

In some aspects, the present disclosure provides the insight that phagocytosis-promoting C3 degradation products can accumulate to considerable levels on the surface of self cells without triggering phagocytosis and may play a significant part in the pathogenesis of chronic inflammatory and autoimmune disorders. Such degradation products can be highly persistent and may serve as an important barometer of danger to resident macrophages. Unlike the anaphylatoxins produced during complement activation, C3b and C3b degradation products such as iC3b, C3dg, and C3d are covalently bound to the cell surface and do not diffuse away. Without wishing to be bound by any theory, accumulation of such C3 degradation products (also referred to as C3 fragments) on self cells may serve as a master switch for many chronic inflammatory diseases.

In some aspects, described herein is the existence of a threshold phenomenon, in that phagocytosis of self cells that become opsonized with C3b fragments is triggered above a certain level of C3b fragment accumulation but rarely occurs before. Self cells that have sub-threshold level of C3 fragments (e.g., iC3b, C3dg, and/or C3d) on their surface may serve as a persistent source of ligands for receptors on the surface of resident immune cells, e.g., resident macrophages or microglia and/or macrophages that enter the tissue or develop from monocytes that enter the tissue under pathologic conditions, e.g., in the setting of inflammation or tissue injury. Cell surface C3 fragments may activate such phagocytic cells, e.g., resident macrophages or microglia, by interacting with these receptors, causing them to mount a response that may result in self cell damage. C3 degradation products that are covalently attached to surfaces serve as ligands for five transmembrane complement receptors: CR1, CR2, CR3, CR4, and CRIg, of which CR2, CR3, and CR4 are of particular interest herein. CR2 is a member of the RCA family and binds to iC3b, C3dg, and C3d via its first two CCP domains. The binding site for CR2 lies within the TED domain, which is present in all of these C3 fragments. In humans, CR2 is present, e.g., on the surface of follicular dendritic cells and B cells. On B cells it forms a complex (CD21/CD19/CD81), which acts as a co-receptor for the B cell receptor (BCR), lowering the threshold for B cell activation and enhancing B cell responses. Complement receptor 3 (CR3) and complement receptor 4 (CR4) are heterodimeric proteins that are members of the β2 family of integrins. CR3 is composed of two subunits: CD11b (also known as integrin subunit α_{M}; Uniprot entry for the human protein is P11215) and CD18 (also known as integrin subunit α₂; Uniprot entry for the human protein is P05107). CR3 is found on monocytes, macrophages, PMNs, NK cells, and certain T and B cell populations. Notably, CR3 is expressed on microglia. CR3 binds to iC3b, C3dg, and C3d as well as a variety of other ligands. It is the main complement receptor involved in phagocytosis of cells and particles bearing C3 fragments on their surface. Complement receptor 4 (CR4) is composed of two subunits, namely CD11c (also known as integrin subunit ax; Uniprot entry for the human protein is P05107) and CD18. CR4 is found on most human dendritic cells and also on monocytes, macrophages, neutrophils, and some T and B cell populations. CR4 binds to the C3c portion of iC3b and facilitates phagocytosis.

Engagement of complement receptors may lead to macrophages/microglial polarization towards a tissue-destructive, pro-inflammatory phenotype. Macrophage polarization refers to the process whereby macrophages express different phenotypes and associated functional programs in response to microenvironmental signals. Different macrophage phenotypes can be defined based on changes in expression of cytokines, receptors and other markers as a response to different classes of stimuli. Macrophage functional states range from the phenotype referred to as M1 (classically activated macrophages) to M2 (alternatively activated/anti-inflammatory). M1 macrophages can produce reactive oxygen species and proinflammatory cytokines such as tumor necrosis factor (TNF) alpha and interleukin (IL)-1β. M2 macrophages encompass a continuum of functional states, which include functions such as secretion of anti-inflammatory cytokines and involvement in wound healing and tissue remodeling and repair.

Macrophages and microglia that are polarized towards a pro-inflammatory phenotype through interactions with phagocytosis-promoting C3 fragments may contribute to tissue damage either directly or indirectly, e.g., through release of molecules that contribute to tissue damage through recruitment of other immune cells. Self cells that go on to accumulate a threshold level of phagocytosis-promoting C3 fragments on their surface may be phagocytosed by resident macrophages/microglia, thereby directly contributing to tissue destruction. These pathogenic mechanisms may play a role in a wide variety of chronic disorders. For example, phagocytosis-promoting C3 fragments may accumulate and persist on retinal cells (e.g., photoreceptors, retinal pigment epithelial cells). C3 fragment accumulation on retinal cells may lead to polarization of resident microglia towards a tissue-destructive, pro-inflammatory phenotype, thereby contributing to the pathogenesis of age-related macular degeneration. Retinal cells that go on to accumulate a threshold level of C3 fragments on their surface may be phagocytosed by, e.g., resident macrophages and/or microglia. Such phagocytosis may contribute directly towards tissue destruction in eyes suffering from age-related macular degeneration, e.g., geographic atrophy. In some embodiments an iC3b/C3dg/C3d inhibitor inhibits phagocytosis of self cells. For example, in some embodiments an iC3b/C3dg/C3d inhibitor inhibits phagocytosis of retinal cells in a subject suffering from an eye disorder, e.g., AMD. In some embodiments the subject with AMD has geographic atrophy.

In some aspects, inhibiting binding of one or more phagocytosis-promoting C3 fragments to CR3 and/or CR4 expressed by macrophage/microglia inhibits their polarization towards a pro-inflammatory, tissue destructive phenotype. In some aspects, inhibiting binding of one or more phagocytosis-promoting C3 fragments to CR2 expressed by B cells or FDCs may contribute indirectly to inhibition of macrophage/microglia polarization towards a pro-inflammatory, tissue destructive phenotype.

Without wishing to be bound by any theory, C3 degradation product accumulation on self cells may occur via a variety of mechanisms. In some instances, self cells may have a deficit in their ability to remove C3 degradation products that are produced via normal “tickover” of complement from their surfaces. In other words, the cells may not maintain the normal homeostatic balance between C3 fragment deposition and removal, thus leading to progressive accumulation of C3 fragments on the cell surface. Without wishing to be bound by any theory, a deficit in a cell's ability to remove C3 degradation products from its surface may result from (i) decreased expression of complement regulatory proteins, which would allow cell-bound C3b to remain active for longer than normal and have a greater chance of participating in convertase formation, resulting in increased level of C3 degradation products on the cell surface; (ii) decreased plasma membrane internalization, e.g., decreased endocytosis, which would cause an increase in the average length of time that a C3 degradation product remains on the cell surface. In some instances autoantibodies that activate complement via classical pathway may bind to antigens on cells or tissues in the body, e.g., in blood vessels, skin, eye, nerves, muscle, connective tissue, heart, kidney, thyroid, lungs, or other organs or tissues in the body. The antigens may be normal proteins or lipids or may have undergone modifications such as oxidation. In some embodiments, a subject has neuromyelitis optica and produces an autoantibody (e.g., an IgG autoantibody) to aquaporin 4. In some embodiments, a subject has pemphigoid and produces an autoantibody (e.g., an IgG or IgE autoantibody) to a structural component of the hemidesmosome (e.g., transmembrane collagen XVII (BP180 or BPAG2) and/or plakin family protein BP230 (BPAG1). In some embodiments, complement activation mediated by autoantibodies bound to the surface of self cells results in C3b deposition on such cells, which is often rapidly converted to iC3b, and then to C3dg and C3d. In some embodiments, a chronic complement-mediated disorder is not characterized by autoantibodies and/or immune complexes. In some instances complement may be activated via the lectin pathway by antigens on cells or tissues in the body, e.g., in blood vessels, skin, eye, nerves, muscle, connective tissue, heart, kidney, thyroid, lungs, or other organs or tissues in the body, e.g., neoantigens exposed as a result of injury or modification. In some embodiments, an increased level of C3 fragment deposition on cells may result at least in part from increased sysnthesis and/or activation of C3 relative to normal levels of C3 activation. In some embodiments, C3 may be activated by protein-containing aggregates and/or such aggregates may stimulate production and/or release of C3 from cells. Even a small imbalance between C3b deposition and C3 degradation product removal could result in extensive C3b degradation product accumulation over time. A reduced or otherwise inadequate level of endocytosis may have a variety of underlying causes. For example, inadequate energy supplies may limit the ability of a cell to carry out endocytosis. As known in the art, mitochondria generate most of the cell's supply of adenosine triphosphate (ATP), which is used as a source of chemical energy, through the process known as oxidative phosphorylation. Without wishing to be bound by any theory, mitochondrial dysfunction, which may result at least in part from oxidative stress, may play a role in accumulation of cell surface C3 fragments, e.g., by leading to an endocytosis deficit.

In some aspects, described herein is a method of treating a subject in need of treatment of a disorder associated with C3 fragment accumulation on self cells, the method comprising administering an iC3b/C3dg/C3d inhibitor to the subject. The term “iC3b/C3dg/C3d inhibitor” refers to an agent that inhibits one or more biological activities of one or more of iC3b, C3dg, and C3d. For purposes of the present disclosure, an iC3b/C3dg/C3d inhibitor is not a complement inhibitor described in the section below entitled “Complement Inhibitors”. The term “iC3b/C3dg/C3d inhibitor” encompasses (A) agents that bind to iC3b, C3dg, and/or C3d and inhibit binding of such C3 degradation product to one or more complement receptors, e.g., CR2, CR3, and/or CR4; (B) agents that bind to one or more complement receptors, e.g., CR2, CR3, and/or CR4, and inhibit binding of one or more cell-bound C3degradation products thereto; (C) agents that promote internalization of cell surface C3 degradation product(s), e.g., by endocytosis. In some embodiments, a complement inhibitor, e.g., a C3 inhibitor, such as an agent that inhibit C3 activity or activation (e.g., as described herein), may be used as to treat a complement-mediated disorder, e.g., any of the complement-mediated disorders described herein, either with or without administration of an iC3b/C3dg/C3d inhibitor as described herein.

In general, an iC3b/C3dg/C3d inhibitor may comprise or consist of an antibody, a polypeptide, a peptide, a nucleic acid (e.g., an aptamer), or a small molecule. In some embodiments an iC3b/C3dg/C3d inhibitor comprises an antibody that specifically binds to one or more of iC3b, C3dg, and C3d and inhibits binding of at least one of these C3b degradation products to CR2, CR3, and/or CR4. In some embodiments the antibody binds to iC3b, C3dg, or both. In some embodiments the antibody binds to iC3b, C3dg, and C3d. In some embodiments the antibody binds to the TED domain. In some embodiments the antibody binds outside the TED domain. In some embodiments the antibody specifically binds to a neoepitope in iC3b that is absent in C3b and is retained in C3dg. In some embodiments the antibody binds to the C3d portion of C3dg. In some embodiments the antibody does not stabilize a C3 or C5 convertase.

Various antibodies that specifically bind to one or more of iC3b, C3dg, and C3d are known in the art, and a number of such antibodies are commercially available. For example, the following antibodies are commercially available: (1) Mouse anti-iC3b antibody catalog A209 (Quidel Corp., San Diego, Calif.), which reacts with a neoantigen on human iC3b, C3dg and C3g. (Tamerius, J. D., M. Pangburn, et al., Detection of a neoantigen on human iC3b and C3d by monoclonal antibody, J. Immunol. 135:2015, 1985). (2) Mouse anti-iC3b monoclonal antibody, clone 013III-1.16 (product code MCA2607, Bio-Rad Antibodies), which reacts with a neoepitope on human iC3b. (3) Rat monocolonal antibody 9 (also known as YB2/90-5-20) (product code HM2199, Hycult Biotech), which reacts with a neoantigen on iC3, iC3b, C3dg and C3g and does not recognize C3 or C3b (described in Lachmann, P et al (1982) J Exp Med, 156: 205; (4) Rat monocolonal antibody 3 (also known as also known as YB2/39-11-1-7) (product code HM2198, Hycult Biotech), which reacts with react with a linear determinant in C3d that is found on C3, C3b, iC3b, C3dg and C3d; (5) clone 1149, Thermo Scientific). Monoclonal antibodies that react with a neoantigen on human iC3b, C3dg and C3g and do not recognize C3 or C3b, and methods of generating such antibodies, are described in Thurman, J M, et al., J Clin Invest. 123(5): 2218-2230 (2013). A rat antihuman C3d monoclonal antibody with specificity to the end sequence of the N-terminal region of C3d is described in Rasmussen K J, et al., J Immunol Methods (2017) 444:51-55. The antibody reportedly can only bind to C3d when it is present as the final end product of cleaved C3. One of ordinary skill in the art could readily generate additional antibodies that bind to one or more C3b degradation products using standard methods. An antibody of non-human origin may be humanized using widely available methods that are routine in the art. Antibody fragments, e.g., scFv antibody fragments, may be generated.

In some embodiments an antibody described in WO2012139069 as binding to iC3b may be used in a composition or method described herein. In some embodiments a molecule described in WO2012139069 as binding to iC3b is not used in a composition or method described herein. In some embodiments an antibody described in WO2011163412 as binding to C3d may be used in a composition or method described herein. In some embodiments a molecule described in WO2011163412 as binding to C3d is not used in a composition or method described herein.

In some embodiments an iC3b/C3dg/C3d inhibitor comprises an antibody that binds to a target receptor (CR2, CR3, and/or CR4) and inhibits binding of its target receptor to one or more C3 degradation products. In some embodiments the antibody binds specifically to a region of CR2, CR3, or CR4 that is involved in interactions with iC3b (e.g., the C3d portion of iC3b) and inhibits such interactions.

In some embodiments an antibody that binds to one or more of iC3b, C3dg, and C3d or that binds to one or more CRs lacks or substantially lacks the ability to promote one or more effector functions, e.g., antibody-dependent cell-mediated cytotoxicity (ADCC), antibody-dependent phagocytosis (ADP), complement activation, and/or complement-dependent cytotoxicity (CDC).

Peptides that bind specifically to a target of interest, e.g., iC3b, C3dg, and/or C3d may be identified using any of a variety of display technologies, such as phage display, yeast display, bacterial display, ribosome display, or mRNA display. Such technologies may additionally or alternately be used to identify peptides that bind to CR2, CR3, and/or CR4. Identified peptides can be further tested to determine their ability to inhibit interaction between iC3b, C3dg, and/or C3d and CR2, CR3, and/or CR4.

A variety of engineered binding proteins that comprise a non-immunoglobulin scaffold (referred to as “non-antibody polypeptides”) may be used as specific binding agents in certain embodiments. For example, anticalins are binding proteins that are constructed on the basis of lipocalins as a scaffold (see, e.g., Skerra, J., J Biotechnol., 74(4):257-75, 2001). Affibodies are binding proteins generated from combinatorial libraries constructed using the protein A-derived Z domain as a scaffold (see, e.g., Nord K, Eur J Biochem., 268(15):4269-77, 2001). Adnectins, also referred to as monobodies, are synthetic binding proteins that are constructed using a fibronectin type III domain (FN3) as a molecular scaffold (see, e.g., Koide S, et al. (2012). “Target-binding proteins based on the 10th human fibronectin type III domain ((10)Fn3)”. Meth. Enzymol. 503: 135-56. Monobodies are generated from combinatorial libraries in which portions of the FN3 scaffold are diversified using molecular display and directed evolution technologies such as phage display, mRNA display and yeast surface display.

In some embodiments an iC3b/C3dg/C3d inhibitor comprises a polypeptide that is at least 90%, 91%, 92%, 93%, 94%, 95%, 96%, 97%, 98%, 99%, 99.5%, or 100% identical to the minimal portion of the extracellular domain (ECD) of CR1, CR2, or CRIg that binds to one or more C3 degradation product(s). The polypeptide blocks binding of one or more of such C3 degradation product(s) to one or more of CR2, CR3, and CR4. In some embodiments the polypeptide is at least 90%, 91%, 92%, 93%, 94%, 95%, 96%, 97%, 98%, 99%, 99.5%, or 100% identical to the minimal portion of the ECD of CR1 that binds to iC3b.

In some embodiments the polypeptide is at least 90%, 91%, 92%, 93%, 94%, 95%, 96%, 97%, 98%, 99%, 99.5%, or 100% identical to the minimal portion of the ECD of CR2 that binds to C3d. In some embodiments the polypeptide comprises at least the portion of CR2 that contains short consensus repeats 1 and 2 (e.g., amino acids 21-147 or 21-148). In some embodiments the polypeptide comprises one or more additional amino acids found C-terminal to amino acid 148 in human CR2. For example, the polypeptide may comprise amino acids 21-149, 21-150, 21-151, 21-152, 21-153, 21-154, 21-155, etc., of human CR2. In some embodiments the polypeptide comprises up to an additional 10, 20, 30, 40, 50, 60, or 70 amino acids found C-terminal to amino acid 148 in human CR2. In some embodiments the polypeptide comprises at least the portion of CR2 that contains SCRs 1, 2, and 3 (e.g., amino acids 21-211 or 21-212). In some embodiments the polypeptide comprises up to an additional 10, 20, 30, 40, 50, 60, or 70 amino acids found C-terminal to amino acid 212 in human CR2. In some embodiments the polypeptide comprises amino acids 21-269 of human CR2.

In some embodiments the polypeptide is at least 90%, 91%, 92%, 93%, 94%, 95%, 96%, 97%, 98%, 99%, 99.5%, or 100% identical to the minimal portion of human α_{M} or α_{X} that binds to iC3b, C3dg, and/or C3d. In some embodiments the polypeptide is at least 90%, 91%, 92%, 93%, 94%, 95%, 96%, 97%, 98%, 99%, 99.5%, or 100% identical to the αI domain of α_{M} (residues 146-334 of UniProt entry P11215). In some embodiments the polypeptide is at least 90%, 91%, 92%, 93%, 94%, 95%, 96%, 97%, 98%, 99%, 99.5%, or 100% identical to the I domain of ax (residues E130-G319 of UniProt entry P20702). In some embodiments the polypeptide contains one or more substitutions relative to the naturally occurring sequence of an αI domain. For example, in some embodiments amino acid 1332 of the αI domain of αM is substituted to G (1332G substitution). In some embodiments F273 of the αI domain of ax is substituted to S and/or F300 of the αI domain of axis substituted to A (F273S and F300A substitutions). In some embodiments analogous substitutions are made at corresponding residues of the αI domain of α_{M}. In some embodiments the substitution(s) increase the stability of the polypeptide in a form that binds to iC3b and/or increase its binding affinity for iC3b. In some embodiments random mutagenesis, optionally followed by directed evolution, may be used to identify substitutions that further increase the stability, binding affinity, and/or specificity of the polypeptide.

In some embodiments the polypeptide is at least 90%, 91%, 92%, 93%, 94%, 95%, 96%, 97%, 98%, 99%, 99.5%, or 100% identical to the minimal portion of human CRIg that binds to iC3b.

In some embodiments the polypeptide comprises two or more portions each at least 90%, 91%, 92%, 93%, 94%, 95%, 96%, 97%, 98%, 99%, 99.5%, or 100% identical to a portion of a human CR, e.g., CR1 and CR2, or CR1 and CR3, or CR2 and CR3, or CR2 and CRIg, wherein the portions are selected such that the polypeptide binds to iC3b, C3dg, and C3d. The portions may be separated by a flexible linking region such that each portion can fold independently and assume the proper conformation for binding to its target.

Nucleic acids may be used as specific binding agents in certain embodiments. Nucleic acid ligands, sometimes referred to as aptamers, are oligonucleotides that bind specifically and with high affinity to a target. They can comprise DNA, RNA, and/or non-standard nucleotides, e.g., 2′-fluoro or 2′-O-methyl modified nucleotides, and may be identified using, e.g., systematic evolution of ligands by exponential enrichment (SELEX) or various directed evolution techniques that are known in the art. See, e.g., Tuerk, C. and Gold, L., Science 249(4968): 505-10, 1990; Brody E N, Gold L. J Biotechnol., 74(1):5-13, 2000.

In some embodiments an iC3b/C3dg/C3d inhibitor binds to one or more of CR2, CR3, and CR4 and competes with one or more cell-bound C3b degradation products for binding to one or more such CR. The iC3b/C3dg/C3d inhibitor that binds to CR2, CR3, and/or CR4 may comprise an antibody, polypeptide, nucleic acid (e.g., aptamer), or small molecule. In some embodiments the iC3b/C3dg/C3d inhibitor comprises a polypeptide at least 90%, 91%, 92%, 93%, 94%, 95%, 96%, 97%, 98%, 99%, 99.5%, or 100% identical to a CR-binding portion of iC3b, C3dg, or C3d. In some embodiments, binding of the iC3b/C3dg/C3d inhibitor to CR2, CR3, and/or CR4 does not mimic the effect of binding of the natural endogenous ligand in regard to its effect on cells expressing CR2, CR3, and/or CR4.

The a subunits of CR3 (α_{M}β₂) and CR4 (α_{X}β₂) contain a domain referred to as an “inserted domain” (αI domain), which plays a key role in recognition of iC3b by both receptors. In some aspects, structural information derived from studies of CR3 or a portion thereof (e.g., the ectodomain, alpha chain, or αI domain) may be used to identify agents that bind to CR3 and inhibit binding of one or more C3 degradation products thereto. The crystal structure of the CR3 αI domain in complex with C3d has been determined (Bajic, G., et al., (2013) PNAS, 110(41): 16426-16431). Residues involved in the CR3:C3d interaction have been identified. In some embodiments an iC3b/C3dg/C3d inhibitor specifically binds to the binding site for CR3 on C3d or specifically binds to the binding site for C3d on CR3. In some embodiments, such agents may be identified through screening, structure-based design, or a combination thereof.

In some aspects, structural information derived from studies of CR4 or a portion thereof (e.g., the ectodomain or alpha chain) may be used to identify agents that bind to CR4 and inhibit binding of one or more C3b degradation products thereto. The crystal structure of a complete CR4 (α_{X}β₂) ectodomain is known in the art (Xie C, et al. (2010) Structure of an integrin with an alphaI domain, complement receptor type 4. EMBO J 29:666-679. Studies of the intact ectodomain of CR4 identified a major binding site for this receptor involving the MG3 and MG4 domains of C3c. The orientation of these domains, and hence the binding interface for CR4, are conserved between C3b and C3c, meaning that this likely to be the case for iC3b also. In some embodiments an iC3b/C3dg/C3d inhibitor specifically binds to the binding site for CR4 on iC3b or specifically binds to the binding site for iC3b on CR4. In some embodiments, such agents may be identified through screening, structure-based design, or a combination thereof.

Studies of CR3 (α_{M}β₂) and CR4 (α_{X}β₂) in complex with iC3b revealed that α_{X}β₂ binds to two distinct sites on iC3b (both within the C3c moiety), and a single α_{M}β₂ integrin binds to two different sites on iC3b (one in the C3d moiety and one in the C3c moiety that is distinct from those to which α_{X}β₂ binds) (Xu, S. et al., (2017) PNAS, 114(13): 3403-3408). It was also found that the primary binding site of α_{X}β₂ and the β_{M}β₂ binding site appear to be sufficiently distinct such that no overlap between α_{X}β₂ and α_{M}β₂ would occur upon their simultaneous binding to the same iC3b molecule.

In certain embodiments, binding of CR3 to both of its binding sites on iC3b is inhibited. In some embodiments this may be accomplished using an agent that binds to iC3b and inhibits binding of CR3 to both of its binding sites. In some embodiments the agent is a bifunctional agent that binds to both binding sites. In some embodiments binding of CR3 to both of its binding sites on iC3b is inhibited using an agent that binds to CR3 and inhibits its binding to both of its binding sites on iC3b.

In certain embodiments, binding of CR4 to both of its binding sites on iC3b is inhibited. In some embodiments this may be accomplished using an agent that binds to iC3b and inhibits binding of CR4 to both of its binding sites. In some embodiments the agent is a bifunctional agent that binds to both binding sites. In some embodiments binding of CR4 to both of its binding sites on iC3b is inhibited using an agent that binds to CR4 and inhibits its binding to both of its binding sites on iC3b.

In certain embodiments binding of both CR3 and CR4 to iC3b is inhibited. In some embodiments this may be accomplished using an agent that binds to iC3b and inhibits binding of both CR3 and CR4 thereto. The agent may, for example, sterically hinder binding of both CR3 and CR4 or cause a change in conformation of iC3b that alters the structure of binding sites for both CR3 and CR4. In some embodiments binding of both CR3 and CR4 to iC3b is inhibited using two distinct agents, one of which inhibits binding of CR3 and the other of which inhibits binding of CR4. In some embodiments the agent(s) are antibodies. In some embodiments the agent(s) are non-antibody polypeptides.

The present disclosure provides embodiments in which each type of specific binding agent described herein (e.g., antibodies, non-antibody polypeptides, nucleic acids, small molecules) is employed as an iC3b/C3dg/C3d inhibitor. Such agents may bind to iC3b, C3dg, and/or C3d or to CR2, CR3, and/or CR4.

The ability of an agent to inhibit binding of a C3 degradation product to one or more CRs may be measured by contacting the C3 degradation product and the CR with the agent, measuring binding of the C3 degradation product to the CR, and comparing the amount of binding the C3 degradation product to the CR with the amount of binding that occurs in the absence of the agent. If the amount of binding of the C3b degradation product to the CR that occurs in the presence of the agent is less than the amount of binding that occurs in the absence of the agent, then the agent inhibits binding of the C3 degradation product to the CR. In some embodiments the C3 degradation product and the CR may be contacted in vitro as isolated proteins in solution. In some embodiments, the C3b degradation product, the CR, or both, is covalently or noncovalently attached to a solid support, e.g., beads (which may be made at least in part of agarose, polystyrene, or other suitable material). In some embodiments cells expressing one or more CRs may be contacted with isolated C3b degradation product in vitro. In some embodiments cells having one or more C3b degradation product on their surface may be contacted with an isolated CR in solution. In some embodiments, cells having one or more C3 degradation product on their surface may be contacted with cells expressing one or more CRs. In some embodiments, any of the methods relating to a CR and a C3 degradation product may utilize a portion of the CR that retains ability to bind to the C3 degradation product. For example, comprising the ectodomain of the relevant CR may be used. In some embodiments a cell expresses only a single CR of interest (e.g., CR2, CR3, or CR4). The cell may naturally express only the CR of interest or may be a genetically modified cell.

In some embodiments a polypeptide described in WO2010033868 as binding to CR2 may be used in a composition or method described herein. In some embodiments a polypeptide described in WO2010033868 described as binding to CR2 is not used in a composition or method described herein.

In some embodiments an iC3b/C3dg/C3d inhibitor is a small molecule. Small molecules, e.g., from compound libraries, may be screened to identify iC3b/C3dg/C3d inhibitors. In some embodiments an iC3b/C3dg/C3d inhibitor is a statin, e.g., simvastatin, atorvastatin, fluvastatin, lovastatin, pitavastatin, pravastatin, or rosuvastatin. In some embodiments a compound used in a composition or method described herein is not a statin. In some embodiments a molecule described in WO2016179057 as binding to C3d may be used in a composition or method described herein. In some embodiments a molecule described in WO2016179057 as binding to C3d is not used in a composition or method described herein.

A structure generated at least in part using, e.g., nuclear magnetic resonance, homology modeling, and/or X-ray crystallography may be used. In some embodiments a structure obtained with a ligand bound to a receptor may be used. In some embodiments a computer-aided computational approach sometimes referred to as “virtual screening” is used in the identification of candidate iC3b/C3dg/C3d inhibitors. Structures of compounds, e.g., small molecules may be screened for ability to bind to a region (e.g., a “pocket”) accessible to the compound and located at an appropriate position to inhibit interaction between iC3b, C3dg, or C3d, and CR2, CR3, or CR4. A variety of docking and pharmacophore-based algorithms are known in the art, and computer programs implementing such algorithms are available. Commonly used programs include Gold, Dock, Glide, FlexX, Fred, and LigandFit (including the most recent releases thereof). See, e.g., Ghosh, S., et al., Current Opinion in Chemical Biology, 10(3): 194-2-2, 2006; McInnes C, Current Opinion in Chemical Biology; 11 (5): 494-502, 2007, and references in either of the foregoing articles, which are incorporated herein by reference. In some embodiments a virtual screening algorithm may involve two major phases: searching (also called “docking”) and scoring. During the first phase, the program automatically generates a set of candidate complexes of two molecules (test compound and target molecule) and determines the energy of interaction of the candidate complexes. The scoring phase assigns scores to the candidate complexes and selects a structure that displays favorable interactions based at least in part on the energy. To perform virtual screening, this process may be repeated with a large number of test compounds to identify those that, for example, display the most favorable interactions with the target. In some embodiments, low-energy binding modes of a small molecule within an active site or possible active site or other target region are identified. In some embodiments a compound capable of docking at a site where mutations are known to inhibit activity of the target is identified. Variations may include the use of rigid or flexible docking algorithms and/or including the potential binding of water molecules. In some embodiments the three-dimensional structure of an enzyme's active site may be used to identify potential inhibitors. Agent(s) that have the potential to bind in or near an active site may be identified. These predictions may then be tested using the actual compound. A new inhibitor thus identified may then be used to obtain a structure of the enzyme in an inhibitor/enzyme complex to show how the molecule is binding to the active site. Further changes may be made to the inhibitor, e.g., to try to improve binding. This cycle may be repeated until an inhibitor of sufficient predicted or actual potency (e.g., a desired potency for therapeutic purposes) is identified.

Numerous small molecule structures are available and can be used for virtual screening. A collection of compound structures may sometimes referred to as a “virtual library”. For example, ZINC is a publicly available database containing structures of millions of commercially available compounds that can be used for virtual screening (http://zinc.docking.org/ or http://zinc15.docking.org; Shoichet, J. Chem. Inf. Model., 45(1): 177-82, 2005; Sterling and Irwin, (2015) J. Chem. Inf Model, 55 (11), pp 2324-2337). A database containing about 250,000 small molecule structures is available on the National Cancer Institute (U.S.) website. In some embodiments multiple small molecules may be screened, e.g., up to 50,000; 100,000; 250,000; 500,000, or up to 1 million, 2 million, 5 million, 10 million, or more. Compounds can be scored and, optionally, ranked by their potential to bind to a target. Compounds identified in virtual screens can be tested in cell-free or cell-based assays or in animal models to confirm their ability to inhibit activity of a target molecule and/or to assess their biological and/or pharmacological activity. Computational approaches may be used to predict one or more physico-chemical, pharmacokinetic and/or pharmacodynamic properties of compounds identified in a physical or virtual screen. Such information may be used, e.g., to select one or more hits for, e.g., further testing, development, or use. For example, small molecules having characteristics typical of “drug-like” molecules may be selected and/or small molecules having one or more undesired characteristics may be avoided.

In some embodiments an iC3b/C3dg/C3d inhibitor binds to its target (e.g., iC3b, C3dg, C3d, CR2, CR3, or CR4) with a K_{d} of between 0.0001 μM and 1.0 μM, e.g., between 0.1 μM and 1.0 μM, between 0.01 μM and 0.1 μM, between 0.001 μM and 0.01 μM, between 0.0001 μM and 0.001 μM, between 0.0001 μM and 0.001 μM, or less. In some embodiments a method comprises screening to identify a compound that binds to iC3b, C3dg, C3d, CR2, CR3, or CR4 with a K_{d} within any of the aforementioned ranges (or less).

In some embodiments an iC3b/C3dg/C3d inhibitor has an IC₅₀ of 10 mM or less for inhibiting one or more biological activities of a C3 degradation product. For example, in some embodiments an iC3b/C3dg/C3d inhibitor has an IC₅₀ of 10 mM or less for inhibiting binding of a C3 degradation product to CR2, CR3, or CR4, or a portion of CR2, CR3, or CR4 that comprises a binding site for the C3 degradation product. The IC₅₀ for inhibiting binding is the concentration of the iC3b/C3dg/C3d inhibitor that reduces binding of the C3 degradation product to the CR by 50%. The IC₅₀ may be measured using a competition binding assay. It will be appreciated that the natural ligand (iC3b, C3dg, or C3d) is usually used at a low concentration relative to the concentration of the CR, usually at or below its K_{d} value. In some embodiments the IC₅₀ is between 1 μm and 10 μm. In some embodiments the IC₅₀ is between 0.1 μm and 1.0 μm. In some embodiments the IC₅₀ is between 0.01 μM and 0.1 μM. In some embodiments the IC₅₀ is between 0.001 μM and 0.01 μM. In some embodiments the IC₅₀ is between 0.0001 μM and 0.001 μM. In some embodiments the IC₅₀ is less than 0.0001 μM. In some embodiments the biological activity of a C3 degradation product is measured as an effect on macrophages or microglia.

In certain embodiments an iC3b/C3dg/C3d inhibitor selectively inhibits binding of a C3 degradation product to CR3 relative to its ability to inhibit binding of such degradation product to CR2. In certain embodiments an iC3b/C3dg/C3d inhibitor selectively inhibits binding of a C3 degradation product to CR4 relative to its ability to inhibit binding of such degradation product to CR2. In certain embodiments an iC3b/C3dg/C3d inhibitor selectively inhibits binding of a C3 degradation product to CR3 relative to its ability to inhibit binding of such degradation product to CR2 and selectively inhibits binding of a C3 degradation product to CR4 relative to its ability to inhibit binding of such degradation product to CR2. In certain embodiments an iC3b/C3dg/C3d inhibitor selectively inhibits binding of a C3 degradation product to CR3 relative to its ability to inhibit binding of such degradation product to CR4. In certain embodiments an iC3b/C3dg/C3d inhibitor selectively inhibits binding of a C3 degradation product to CR4 relative to its ability to inhibit binding of such degradation product to CR3. In certain embodiments an iC3b/C3dg/C3d inhibitor selectively inhibits binding of a C3 degradation product to CR3 and/or CR4 relative to its ability to inhibit binding of such degradation product to CR1. In certain embodiments an iC3b/C3dg/C3d inhibitor selectively inhibits binding of a C3 degradation product to CR3 and/or CR4 relative to its ability to inhibit binding of such degradation product to CRIg. Selectivity may be measured as relative IC50, where an agent is selective for inhibiting binding of a first CR relative to a second CR if the IC50 for inhibiting binding to the first CR is less than the IC50 for inhibiting binding to the second CR by a factor of at least 2, e.g., between 2 and 5, between 5 and 10, between 10 and 100, between 100 and 1000, between 1000 and 10,000, or more.

In some embodiments the K_{d} of an iC3b/C3dg/C3d inhibitor for binding to iC3b, C3dg, or C3d is at least 10-fold, at least 50-fold, at least 100-fold, or at least 1000-fold lower than its K_{d} for binding to C3b. In some embodiments the K_{d} of an iC3b/C3dg/C3d inhibitor for binding to iC3b, C3dg, or C3d is at least 10-fold, at least 50-fold, at least 100-fold, or at least 1000-fold lower than its K_{d} for binding to C3. In some embodiments an iC3b/C3dg/C3d inhibitor does not detectably bind to C3. In some embodiments an iC3b/C3dg/C3d inhibitor does not detectably bind to C3b. In some embodiments an iC3b/C3dg/C3d inhibitor does not detectably bind to CRIg.

In some embodiments an iC3b/C3dg/C3d inhibitor does not comprise and is not linked to a detectable label. For example, the iC3b/C3dg/C3d inhibitor does not comprise and is not linked to a radioactive moiety, a fluorophore, a fluorescent polypeptide, a metal atom, a magnetic particle, or a quantum dot. In some aspects, a detectable label may be any moiety that contributes to making an entity detectable but whose absence does not affect the ability of the agent to serve as an iC3b/C3dg/C3d inhibitor.

In some embodiments an iC3b/C3dg/C3d inhibitor does not comprise a complement inhibitor. For example, in some embodiments the iC3b/C3dg/C3d inhibitor does not comprise a complement inhibiting portion of factor H or CD59, e.g., the iC3b/C3dg/C3d inhibitor lacks or substantially lacks the capacity to act as a cofactor for factor I or to accelerate the decay of convertases. “Substantially lacks” in this context means that the molecule has less than 5% of the activity of full length factor H or CD59.

In general, the ability of an agent to bind to a C3 degradation product or CR or to inhibit binding of a C3 degradation product to a CR may be measured using any of a variety of assays commonly used in the art to measure binding interactions between agents and proteins. Many such assays are known to those of ordinary skill in the art, including, e.g., immunoblots, enzyme-linked immunosorbent assays (ELISAs), including competitive ELISAs, radioimmunoassay (RIA), surface plasmon resonance, flow cytometry, fluorescence activated cell sorting (FACS), nuclear magnetic resonance (NMR) spectroscopy, analytical ultracentrifugation, and gel filtration chromatography. In some aspects, such assays may be used to measure the binding affinity of an agent to a C3 degradation product or CR.

In some aspects of any screening and/or characterization methods, test agents are contacted with test cells (and optionally control cells) or used in cell-free assays at a predetermined concentration. In some embodiment the concentration is about up to 1 nM. In some embodiments the concentration is between about 1 nM and about 100 nM. In some embodiments the concentration is between about 100 nM and about 10 μM. In some embodiments the concentration is at or above 10 μM, e.g., between 10 μM and 100 μM. Following incubation for an appropriate time, optionally a predetermined time, the effect of compounds or composition on a parameter of interest in the test cells is determined by an appropriate method known to one of ordinary skill in the art, e.g., as described herein. Cells can be contacted with compounds for various periods of time. In certain embodiments cells are contacted for between 5 minutes and 2 hours, between 2 hours and 12 hours, between 12 hours and 20 days, e.g., for between 1 and 10 days, for between 2 and 5 days, or any intervening range or particular value. Cells can be contacted transiently or continuously. If desired, the compound can be removed prior to assessing the effect on the cells.

An iC3b/C3dg/C3d inhibitor may be used in the treatment of a wide variety of chronic disorders in various embodiments. Generally, a chronic disorder is one that persists for at least 6 months. The terms “disorder” and “disease” are used interchangeably herein. In some embodiments the disorder is a chronic inflammatory disorder, which term encompasses a range of ailments characterized by self-perpetuating immune insults to a variety of tissues and that seem to be dissociated from the initial insult that caused the ailment (which may be unknown).

In some embodiments, the disorder is a chronic eye disorder. In some embodiments, the chronic eye disorder is characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. Macular degeneration related conditions include, e.g., age-related macular degeneration (AMD). Early and intermediate AMD are characterized by the presence of drusen, localized deposits of lipoproteinaceous material that accumulate in the space between the RPE and Bruch's membrane. In some embodiments, a subject has early or intermediate AMD. In some embodiments, a subject is in need of treatment for dry AMD. In some embodiments, a subject is in need of treatment for geographic atrophy (GA), a condition characterized by discrete area(s) of loss of retinal pigment epithelium associated with loss of the overlying photoreceptors. In some embodiments, a subject is in need of treatment for wet AMD. In some embodiments, a subject is in need of treatment for advanced AMD (geographic atrophy or wet AMD). In some instances, GA and wet AMD may co-exist in the same eye or a subject may have GA in one eye and wet AMD in the other eye. In some embodiments, compound(s) may be administered to an eye exhibiting drusen, e.g., to inhibit progression to advanced AMD. In some embodiments, compound(s) may be administered to an eye exhibiting GA, wet AMD, or both.

In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis.

In some embodiments the disorder affects the nervous system, e.g., the central nervous system (CNS) and/or peripheral nervous system (PNS). Examples of such disorders include, e.g., multiple sclerosis, neuromyelitis optica and other chronic demyelinating diseases, amyotrophic lateral sclerosis, chronic pain, stroke, allergic neuritis, Huntington's disease, Alzheimer's disease, and Parkinson's disease. In some embodiments the disorder affecting the nervous system is a neurodegenerative disease, i.e., a disease characterized by progressive loss of structure and/or function of neurons, typically including or progessing to death of neurons. Examples of such diseases include amyotrophic lateral sclerosis, Huntington's disease, Alzheimer's disease, and Parkinson's disease. In some embodiments the disorder is traumatic brain injury, spinal cord injury, or stroke (e.g., ischemic stroke). In some embodiments, described herein is a method of treating any of the foregoing disorders affecting the nervous system, the method comprising administering an iC3b/C3dg/C3d inhibitor to a subject in need of treatment for the disorder. In some embodiments the disorder is a neuropsychiatric disorder such as depression or schizophrenia.

In some embodiments, the disorder is a chronic respiratory disorder. In some embodiments, a chronic respiratory disorder is asthma or chronic obstructive pulmonary disease (COPD). In some embodiments, a chronic respiratory disorder is pulmonary fibrosis (e.g., idiopathic pulmonary fibrosis), radiation-induced lung injury, allergic bronchopulmonary aspergillosis, hypersensitivity pneumonitis (also known as allergic alveolitis), eosinophilic pneumonia, interstitial pneumonia, sarcoid, Wegener's granulomatosis, or bronchiolitis obliterans.

In some embodiments the disorder is allergic rhinitis, chronic rhinosinusitis, or nasal polyposis. In some embodiments described herein is a method of treating a subject in need of treatment for allergic rhinitis, chronic rhinosinusitis (CRS), or nasal polyposis, the method comprising administering an iC3b/C3dg/C3d inhibitor to a subject in need of treatment for the disorder. In some embodiments the disorder is CRS with nasal polyposis. In some embodiments the disorder is CRS without nasal polyposis. In some embodiments the disorder is nasal polyposis and the subject does not have CRS.

In some embodiments, the disorder affects the musculoskeletal system. Examples of such disorders include inflammatory joint conditions (e.g., arthritis such as rheumatoid arthritis or psoriatic arthritis, juvenile chronic arthritis, spondyloarthropathies Reiter's syndrome, gout). In some embodiments, a musculoskeletal system disorder results in symptoms such as pain, stiffness and/or limitation of motion of the affected body part(s). Inflammatory myopathies include dermatomyositis, polymyositis, and various others are disorders of chronic muscle inflammation of unknown etiology that result in muscle weakness. In some embodiments, the disorder is myasthenia gravis. In some embodiments, described herein is method of treating any of the foregoing disorders affecting the musculoskeletal system, the method comprising administering an iC3b/C3dg/C3d inhibitor to a subject in need of treatment for the disorder.

In some embodiments the disorder is a complement-mediated hemolytic condition, e.g., paroxysmal nocturnal hemoglobinuria or autoimmune hemolytic anemia or the HELLP syndrome or atypical hemolytis uremic syndrome.

In some embodiments, the disorder affects the integumentary system. Examples of such disorders include, e.g., pemphigoid, atopic dermatitis, psoriasis, pemphigus, systemic lupus erythematosus, dermatomyositis, scleroderma, sclerodermatomyositis, Sjogren syndrome, and chronic urticaria. In some aspects, described herein is a method of treating any of the foregoing disorders affecting the integumentary system, the method comprising administering an iC3b/C3dg/C3d inhibitor to a subject in need of treatment for the disorder.

In some embodiments the disorder affects the circulatory system. For example, in some embodiments the disorder is a vasculitis or other disorder associated with vessel inflammation, e.g., blood vessel and/or lymph vessel inflammation. In some embodiments, a vasculitis is polyarteritis nodosa, Wegener's granulomatosis, giant cell arteritis, Churg-Strauss syndrome, microscopic polyangiitis, Henoch-Schonlein purpura, Takayasu's arteritis, Kawasaki disease, or Behcet's disease. In some embodiments, a subject, e.g., a subject in need of treatment for vasculitis, is positive for antineutrophil cytoplasmic antibody (ANCA).

In some embodiments the disorder affects the gastrointestinal system. For example, the disorder may be inflammatory bowel disease, e.g., Crohn's disease or ulcerative colitis. In some embodiments, described herein a method of treating an inflammatory disorder that affects the gastrointestinal system, the method comprising administering an iC3b/C3dg/C3d inhibitor to a subject in need of treatment for the disorder.

In some embodiments the disorder is a thyroiditis (e.g., Hashimoto's thryoiditis, Graves' disease, post-partum thryoiditis), myocarditis, hepatitis (e.g., hepatitis C or autoimmune hepatitis), or chronic pancreatitis.

In some embodiments the disorder is a nephropathy (a disorder affecting the kidneys), e.g., IgA nephropathy, lupus nephritis, C3 glomerulopathy. In some embodiments, described herein a method of treating an inflammatory disorder that affects the gastrointestinal system, the method comprising administering an iC3b/C3dg/C3d inhibitor to a subject in need of treatment for the disorder.

In some embodiments described herein is a method of treating a subject suffering from chronic pain, the method comprising administering an iC3b/C3dg/C3d inhibitor to the subject. In some embodiments the subject suffers from chronic inflammatory pain or neuropathic pain. Neuropathic pain has been defined as pain initiated or caused by a primary lesion or dysfunction in the nervous system, in particular, pain arising as a direct consequence of a lesion or disease affecting the somatosensory system. For example, neuropathic pain may arise from lesions that involve the somatosensory pathways with damage to small fibres in peripheral nerves and/or to the spino-thalamocortical system in the CNS. In some embodiments, neuropathic pain arises from autoimmune disease (e.g., multiple sclerosis), metabolic disease (e.g., diabetes), infection (e.g., viral disease such as shingles or HIV), vascular disease (e.g., stroke), trauma (e.g., injury, surgery), or cancer. For example, neuropathic pain can be pain that persists after healing of an injury or after cessation of a stimulus of peripheral nerve endings or pain that arises due to damage to nerves. Exemplary conditions of or associated with neuropathic pain include painful diabetic neuropathy, post-herpetic neuralgia (e.g., pain persisting or recurring at the site of acute herpes zoster 3 or more months after the acute episode), trigeminal neuralgia, cancer related neuropathic pain, chemotherapy-associated neuropathic pain, HIV-related neuropathic pain (e.g., from HIV neuropathy), central/post-stroke neuropathic pain, neuropathy associated with back pain, e.g., low back pain (e.g., from radiculopathy such as spinal root compression, e.g., lumbar root compression, which compression may arise due to disc herniation), spinal stenosis, peripheral nerve injury pain, phantom limb pain, polyneuropathy, spinal cord injury related pain, myelopathy, and multiple sclerosis. Activation of microglial cells has been implicated in the development and perpetuation of chronic pain via mechanisms such as release of inflammatory mediators and neurotransmitters that induce sustained activation of neuronal sensory pathways. In certain embodiments an iC3b/C3dg/C3d inhibitor is administered to treat chronic pain associated with one or more of the afore-mentioned conditions.

In some embodiments an iC3b/C3dg/C3d inhibitor is administered to a subject prior to, during, or after the subject receives a transplant, in order to protect a graft from damage mediated by the subject's immune system. Transplantation is a therapeutic approach of increasing importance, providing a means to replace organs and tissues that have been damaged through trauma, disease, or other conditions. Kidneys, liver, lungs, pancreas, and heart are among the organs that can be successfully transplanted. Tissues that are frequently transplanted include bones, cartilage, tendons, cornea, skin, heart valves, and blood vessels. Pancreatic islet or islet cell transplantation is a promising approach for treatment of diabetes, e.g., type I diabetes. For purposes of the invention, an organ, tissue, or cell (or population of cells) that is be transplanted, is being transplanted, or has been transplanted may be referred to as a “graft”. A graft may have an increased susceptibility to accumulate C3 fragments on its surface relative to native tissues or organs. For example, a graft may contain antigens that result in complement activation or may have diminished endocytotic activity.

In some embodiments the disorder to be treated with an iC3b/C3dg/C3d inhibitor is refractory to one or more standard therapies. “Standard therapy” refers to therapy that is art-accepted as a treatment for the disorder. The therapy is typically approved by a government agency for treatment of at least one disorder but may or may not be approved for treatment of the disorder that is treated with the iC3b/C3dg/C3d inhibitor. In some embodiments the standard therapy comprises steroids. In some embodiments the standard therapy comprises an immunosuppressive agent.

### IV. Combination Therapy with a iC3b/C3dg/C3d Inhibitor and a Complement Inhibitor

Complement inhibition that inhibits activation of C3 (either by acting directly on C3 or acting on one or more complement components upstream of C3) can inhibit generation of C3 fragments. C3 fragments that are already present on self cell surfaces may eventually be removed by plasma membrane internalization (e.g., by endocytosis). Without wishing to be bound by any theory, the presence of sub-threshold amounts of C3 fragments on self cells may delay the achievement of maximum therapeutic efficacy (e.g., the achievement of maximum disease-modifying effect) following initiation of effective treatment with a complement inhibitor that acts on or upstream of C3. Although such complement inhibitors can inhibit further C3 fragment deposition, maximum therapeutic efficacy may, in some instances, not be achieved until cell surface C3b fragments have been largely removed from the plasma membrane, e.g., by endocytosis.

In some aspects, the efficacy of complement inhibitor therapy for treating a disorder may be enhanced by combining such therapy with administration of an iC3b/C3dg/C3d inhibitor. For example, in some embodiments, the efficacy of a C3 inhibitor may be enhanced by combining such therapy with administration of an iC3b/C3dg/C3d inhibitor. In some aspects, the efficacy of an alternative pathway inhibitor may be enhanced by combining such therapy with administration of an iC3b/C3dg/C3d inhibitor. In some embodiments the alternative pathway inhibitor is a factor D inhibitor or factor B inhibitor. In some aspects, the efficacy of a classical pathway inhibitor may be enhanced by combining such therapy with administration of an iC3b/C3dg/C3d inhibitor. In some embodiments the classical pathway inhibitor is a C1s inhibitor. In some aspects, the efficacy of a lectin pathway inhibitor may be enhanced by combining such therapy with administration of an iC3b/C3dg/C3d inhibitor. In some embodiments the lectin pathway inhibitor is a MASP-1 inhibitor, MASP-2 inhibitor, or a MASP-3 inhibitor. In some aspects, the efficacy of a C5 inhibitor may be enhanced by combining such therapy with administration of an iC3b/C3dg/C3d inhibitor and, in some embodiments, a complement inhibitor that inhibits C3 activation. In some embodiments the disorder may be any of the disorders described above.

In some embodiments, a complement inhibitor and an iC3b/C3dg/C3d inhibitor may be administered in combination for a period of at least 3 months, e.g., between 3 months and 6 months, between 6 months and 12 months, between 12 months and 24 months, or more. In some embodiments, treatment with the iC3b/C3dg/C3d inhibitor may be discontinued after a period of time while treatment with the complement inhibitor is continued. In some embodiments, for example, the subject may be treated with both a complement inhibitor and an iC3b/C3dg/C3d inhibitor for a period of at least 3 months, e.g., between 3 months and 6 months, between 6 months and 12 months, or between 12 months and 24 months, at which time treatment with the iC3b/C3dg/C3d inhibitor may be discontinued for a period of at least 6 months, at least 12 months, or more, e.g., indefinitely. In some embodiments, if treatment with two agents is started at different times, the duration of combination therapy with the two agents may be calculated as the time period between the first dose of the agent that is started second and the last dose of the agent that is stopped first. In some embodiments, if treatment with two agents is started at the same time, the duration of combination therapy with the two agents may be calculated as the time period between the first doses of both agents and the last dose of the agent that is stopped first. In some embodiments, treatment with the iC3b/C3dg/C3d inhibitor may continue until the level of iC3b/C3dg/C3d on self cells in a tissue or organ affected by a chronic inflammatory or autoimmune disorder is within normal limits. In some embodiments treatment with the iC3b/C3dg/C3d inhibitor may resume if the subject experiences an episode of excessive complement activation, experiences increased symptoms, and/or exhibits signs of recurrence or worsening of the disorder.

Complement Inhibitors

General

A variety of different complement inhibitors may be used in various embodiments of the compositions and methods described herein. In general, the complement inhibitor may belong to any of various compound classes such as peptides, polypeptides, antibodies (e.g., human or humanized monoclonal antibodies, which may be full size, fragments, single-chain, single domain antibodies, etc.), small molecules, and nucleic acids (e.g., nucleic acid aptamers that bind to a complement component; RNAi agents such as short interfering RNAs that inhibit expression of a complement component by, e.g., causing RNAi-mediated cleavage of mRNA that encodes a complement component or inhibiting translation of such mRNA; antisense oligonucleotides that inhibit expression of a complement component by, e.g., inhibiting translation of such mRNA. In certain embodiments a complement inhibitor comprises an antibody or other specific binding agent that binds to a complement component and, e.g., inhibits its activity or cleavage. In certain embodiments a complement inhibitor inhibits an enzymatic activity of a complement protein. The enzymatic activity may be proteolytic activity, such as ability to cleave another complement protein. In some embodiments, a complement inhibitor inhibits cleavage of C3, C4, C5, or factor B. In some embodiments, a complement inhibitor acts on a complement component that lies upstream of C3 in the complement activation cascade. In some embodiments, a complement inhibitor binds to C3. In some embodiments a complement inhibitor that inhibits C3 activation or activity is used. In some embodiments, a complement inhibitor binds to C4. In some embodiments, a complement inhibitor binds to factor B. In some embodiments, a complement inhibitor binds to factor D. In some embodiments, a complement inhibitor binds to C5. In certain embodiments a complement inhibitor that inhibits at least the classical pathway of complement activation is used. In certain embodiments a complement inhibitor that inhibits at least the alternative pathway of complement activation is used. In certain embodiments a complement inhibitor that inhibits both the classical and the alternative pathway is used. In certain embodiments a complement inhibitor that inhibits the classical pathway, the alternative pathway, and the MBL pathway is used. In some embodiments, a complement inhibitor inhibits activation of at least one complement receptor protein. In certain embodiments the complement receptor protein is a receptor for C3a, e.g., C3aR. In certain embodiments the complement receptor protein is a receptor for C5a, e.g., C5aR or C5L2. In certain embodiments the complement inhibitor does not bind to C5. In certain embodiments the complement inhibitor does not bind to C5aR, C5a, or C5L2. In certain embodiments the complement inhibitor binds to C3a or C3aR. In certain embodiments the complement inhibitor does not bind to C3a or C3aR.

In some embodiments, a complement inhibitor comprises an antibody that substantially lacks the capacity to activate complement. For example, the antibody may have less than 20%, less than 10%, less than 5%, or less than 1% complement stimulating activity as compared with full length human IgG1. In some embodiments, the antibody comprises a CH2 domain that has reduced ability to bind C1q as compared with human IgG1 CH2 domain. In some embodiments, the antibody contains CHI, CH2, and/or CH3 domains from human IgG4 and/or does not contain CHI, CH2, and/or CH3 domains from human IgG1.

In some embodiments, a complement inhibitor has a molecular weight of between 50 Daltons and 1 kilodalton (kD). In some embodiments, a complement inhibitor has a molecular weight between 1 kD and 2 kD, between 2 kD and 5 kD, between 5 kD and 10 kD, between 10 kD and 20 kD, between 20 kD and 30 kD, between 30 kD and 50 kD, between 50 kD and 100 kD, or between 100 kD and 200 kD.

In some embodiments, a complement inhibitor may be at least in part identical to a naturally occurring complement inhibiting agent or a variant or fragment thereof. A variety of different omplement inhibiting polypeptides are produced by viruses (e.g., Poxviruses, Herpesviruses), bacteria (e.g., Staphylococcus), and other microorganisms. Complement inhibiting proteins are produced by various parasites, e.g., ectoparasites, such as ticks. A complement inhibitor can comprise at least a portion of a mammalian complement control or complement regulatory protein or receptor. See Ricklin, D., et al., Nature Biotechnology, 25(11): 1265-75, 2007, for discussion of certain complement inhibitors that are or have been in preclinical or clinical development for various disorders.

In some embodiments a complement inhibitor is used in an amount sufficient to inhibit expression or activity of one or more complement components by at least 20%, 30%, 40%, 50%, 60%, 70%, 80%, 90%, 95%, or 100% relative to a suitable control, e.g., between 50% and 75%, 75% and 90%, or 90% and 100% relative to a suitable control. In some embodiments a complement inhibitor is used in an amount sufficient to inhibit complement activation capacity or complement activation via the classical, alternative, by at least 20%, 30%, 40%, 50%, 60%, 70%, 80%, 90%, 95%, or 100% relative to a suitable control, e.g., between 50% and 75%, 75% and 90%, or 90% and 100% relative to a suitable control.

The following sections further discuss non-limiting exemplary complement inhibitors of use in embodiments of the present invention. Complement inhibitors have been classified in various groups for purposes of convenience. It will be understood that certain complement inhibitors fall into multiple categories.

In some embodiments a complement inhibitor comprises an adnectin, affibody, anticalin, or other type of engineered polypeptide sometimes used in the art in lieu of an antibody, wherein the engineered polypeptide binds to a complement component, e.g., C3, C4, factor B, factor D, or C5.

In some embodiments, a complement inhibitor that binds to substantially the same binding site (e.g., a binding site on a complement component such as C3, C5, factor B, factor D, or an active complement split product) as a complement inhibitor described herein is used. In general, the ability of first and second agents to bind to substantially the same site on a target molecule, such as a complement component or receptor, can be assessed using methods known in the art, such as competition assays, molecular modeling, etc. (See, e.g., discussion of compstatin analog mimetics.) Optionally the first and/or second agent can be labeled with a detectable label, e.g., a radiolabel, fluorescent label, etc. Optionally the target molecule, first agent, or second agent is immobilized on a support, e.g., a slide, filter, chip, beads, etc. In some embodiments, a second antibody that binds to substantially the same binding site as a first antibody comprises one or more CDR(s) that are at least 90%, 91%, 92%, 93%, 94%, 95%, 96%, 97%, 98%, 99%, or 100% identical to CDR(s) of the first antibody.

Compounds that Inhibit C3 Activation or Activity

Compstatin Analogs and Mimetics

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7(3):619-27, 1998) to refer to a peptide having the same sequence as SEQ ID NO: 2 disclosed in U.S. Pat. No. 6,319,897, but amidated at the C terminus as shown in Table 1 (SEQ ID NO: 8). The term “compstatin” is used herein consistently with such usage (i.e., to refer to SEQ ID NO: 8). Compstatin analogs that have higher complement inhibiting activity than compstatin have been developed. See, e.g., WO2004/026328 (PCT/US2003/029653), Morikis, D., et al., Biochem Soc Trans. 32 (Pt 1):28-32, 2004, Mallik, B., et al., J. Med. Chem., 274-286, 2005; Katragadda, M., et al. J. Med. Chem., 49: 4616-4622, 2006; WO2007062249 (PCT/US2006/045539); WO2007044668 (PCT/US2006/039397), WO/2009/046198 (PCT/US2008/078593); WO/2010/127336 (PCT/US2010/033345); US Pat. App. Pub. Nos. 20140113874; WO2015142701 (PCT/US15/20672); US Pat. App. Pub. No. 20170173107. In certain embodiments a compstatin analog described in PCT/US2012/054180 (published as WO/2013/036778) and/or in US Pat. Pub. No. 20150158915 may be used in a composition or method described herein.

Compstatin analogs may be acetylated or amidated, e.g., at the N-terminus and/or C-terminus. For example, compstatin analogs may be acetylated at the N-terminus and amidated at the C-terminus. Consistent with usage in the art, “compstatin” as used herein, and the activities of compstatin analogs described herein relative to that of compstatin, refer to compstatin amidated at the C-terminus (Mallik, 2005, supra).

Concatamers or multimers of compstatin or a complement inhibiting analog thereof are also of use in the present invention.

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

The activity of a compstatin analog may be expressed in terms of its IC₅₀ (the concentration of the compound that inhibits complement activation by 50%), with a lower IC₅₀ indicating a higher activity as recognized in the art. The activity of a preferred compstatin analog for use in the present invention is at least as great as that of compstatin. It is noted that certain modifications known to reduce or eliminate complement inhibiting activity and may be explicitly excluded from any embodiment of the invention. The IC₅₀ of compstatin has been measured as 12 μM using an alternative pathway-mediated erythrocyte lysis assay (WO2004/026328). It will be appreciated that the precise IC₅₀ value measured for a given compstatin analog will vary with experimental conditions (e.g., the serum concentration used in the assay). Comparative values, e.g., obtained from experiments in which IC₅₀ is determined for multiple different compounds under substantially identical conditions, are of use. In one embodiment, the IC₅₀ of the compstatin analog is no more than the IC₅₀ of compstatin. In certain embodiments of the invention the activity of the compstatin analog is between 2 and 99 times that of compstatin (i.e., the analog has an IC₅₀ that is less than the IC₅₀ of compstatin by a factor of between 2 and 99). For example, the activity may be between 10 and 50 times as great as that of compstatin, or between 50 and 99 times as great as that of compstatin. In certain embodiments of the invention the activity of the compstatin analog is between 99 and 264 times that of compstatin. For example, the activity may be 100, 110, 120, 130, 140, 150, 160, 170, 180, 190, 200, 210, 220, 230, 240, 250, 260, or 264 times as great as that of compstatin. In certain embodiments the activity is between 250 and 300, 300 and 350, 350 and 400, or 400 and 500 times as great as that of compstatin. The invention further contemplates compstatin analogs having activities between 500 and 1000 times that of compstatin, or more, e.g., between 1000 and 2000 times that of compstatin, or more. In certain embodiments the IC₅₀ of the compstatin analog is between about 0.2 μM and about 0.5 μM. In certain embodiments the IC₅₀ of the compstatin analog is between about 0.1 μM and about 0.2 μM. In certain embodiments the IC₅₀ of the compstatin analog is between about 0.05 μM and about 0.1 μM. In certain embodiments the IC₅₀ of the compstatin analog is between about 0.001 μM and about 0.05 μM.

The K_{d} of compstatin binding to C3 can be measured using isothermal titration calorimetry (Katragadda, et al., J Biol. Chem., 279(53), 54987-54995, 2004). Binding affinity of a variety of compstatin analogs for C3 has been correlated with their activity, with a lower K_{d} indicating a higher binding affinity, as recognized in the art. A linear correlation between binding affinity and activity was shown for certain analogs tested (Katragadda, 2004, supra; Katragadda 2006, supra). In certain embodiments of the invention the compstatin analog binds to C3 with a K_{d} of between 0.1 μM and 1.0 μM, between 0.05 μM and 0.1 μM, between 0.025 μM and 0.05 μM, between 0.015 μM and 0.025 μM, between 0.01 μM and 0.015 μM, or between 0.001 μM and 0.01 μM.

Compounds “designed or identified based on compstatin” include, but are not limited to, compounds that comprise an amino acid chain whose sequence is obtained by (i) modifying the sequence of compstatin (e.g., replacing one or more amino acids of the sequence of compstatin with a different amino acid or amino acid analog, inserting one or more amino acids or amino acid analogs into the sequence of compstatin, or deleting one or more amino acids from the sequence of compstatin); (ii) selection from a phage display peptide library in which one or more amino acids of compstatin is randomized, and optionally further modified according to method (i); or (iii) identified by screening for compounds that compete with compstatin or any analog thereof obtained by methods (i) or (ii) for binding to C3 or a fragment thereof. Many useful compstatin analogs comprise a hydrophobic cluster, a β-turn, and a disulfide bridge.

In certain embodiments of the invention the sequence of the compstatin analog comprises or consists essentially of a sequence that is obtained by making 1, 2, 3, or 4 substitutions in the sequence of compstatin, i.e., 1, 2, 3, or 4 amino acids in the sequence of compstatin is replaced by a different standard amino acid or by a non-standard amino acid. In certain embodiments of the invention the amino acid at position 4 is altered. In certain embodiments of the invention the amino acid at position 9 is altered. In certain embodiments of the invention the amino acids at positions 4 and 9 are altered. In certain embodiments of the invention only the amino acids at positions 4 and 9 are altered. In certain embodiments of the invention the amino acid at position 4 or 9 is altered, or in certain embodiments both amino acids 4 and 9 are altered, and in addition up to 2 amino acids located at positions selected from 1, 7, 10, 11, and 13 are altered. In certain embodiments of the invention the amino acids at positions 4, 7, and 9 are altered. In certain embodiments of the invention amino acids at position 2, 12, or both are altered, provided that the alteration preserves the ability of the compound to be cyclized. Such alteration(s) at positions 2 and/or 12 may be in addition to the alteration(s) at position 1, 4, 7, 9, 10, 11, and/or 13. Optionally the sequence of any of the compstatin analogs whose sequence is obtained by replacing one or more amino acids of compstatin sequence further includes up to 1, 2, or 3 additional amino acids at the C-terminus. In one embodiment, the additional amino acid is Gly. Optionally the sequence of any of the compstatin analogs whose sequence is obtained by replacing one or more amino acids of compstatin sequence further includes up to 5, or up to 10 additional amino acids at the C-terminus. It should be understood that compstatin analogs may have any one or more of the characteristics or features of the various embodiments described herein, and characteristics or features of any embodiment may additionally characterize any other embodiment described herein, unless otherwise stated or evident from the context. In certain embodiments of the invention the sequence of the compstatin analog comprises or consists essentially of a sequence identical to that of compstatin except at positions corresponding to positions 4 and 9 in the sequence of compstatin.

Compstatin and certain compstatin analogs having somewhat greater activity than compstatin contain only standard amino acids (“standard amino acids” are glycine, leucine, isoleucine, valine, alanine, phenylalanine, tyrosine, tryptophan, aspartic acid, asparagine, glutamic acid, glutamine, cysteine, methionine, arginine, lysine, proline, serine, threonine and histidine). Certain compstatin analogs having improved activity incorporate one or more non-standard amino acids. Useful non-standard amino acids include singly and multiply halogenated (e.g., fluorinated) amino acids, D-amino acids, homo-amino acids, N-alkyl amino acids, dehydroamino acids, aromatic amino acids (other than phenylalanine, tyrosine and tryptophan), ortho-, meta- or para-aminobenzoic acid, phospho-amino acids, methoxylated amino acids, and α,α-disubstituted amino acids. In certain embodiments of the invention, a compstatin analog is designed by replacing one or more L-amino acids in a compstatin analog described elsewhere herein with the corresponding D-amino acid. Such compounds and methods of use thereof are an aspect of the invention. Exemplary non-standard amino acids of use include 2-naphthylalanine (2-NaI), 1-naphthylalanine (1-NaI), 2-indanylglycine carboxylic acid (2Ig1), dihydrotrpytophan (Dht), 4-benzoyl-L-phenylalanine (Bpa), 2-α-aminobutyric acid (2-Abu), 3-α-aminobutyric acid (3-Abu), 4-α-aminobutyric acid (4-Abu), cyclohexylalanine (Cha), homocyclohexylalanine (hCha), 4-fluoro-L-tryptophan (4fW), 5-fluoro-L-tryptophan (5fW), 6-fluoro-L-tryptophan (6fW), 4-hydroxy-L-tryptophan (4OH-W), 5-hydroxy-L-tryptophan (5OH-W), 6-hydroxy-L-tryptophan (6OH-W), 1-methyl-L-tryptophan (1MeW), 4-methyl-L-tryptophan (4MeW), 5-methyl-L-tryptophan (5MeW), 7-aza-L-tryptophan (7aW), α-methyl-L-tryptophan (αMeW), β-methyl-L-tryptophan (βMeW), N-methyl-L-tryptophan (NMeW), ornithine (om), citrulline, norleucine, γ-glutamic acid, etc.

In certain embodiments of the invention the compstatin analog comprises one or more Trp analogs (e.g., at position 4 and/or 7 relative to the sequence of compstatin). Exemplary Trp analogs are mentioned above. See also Beene, et. al. Biochemistry 41: 10262-10269, 2002 (describing, inter alia, singly- and multiply-halogenated Trp analogs); Babitzke & Yanofsky, J. Biol. Chem. 270: 12452-12456, 1995 (describing, inter alia, methylated and halogenated Trp and other Trp and indole analogs); and U.S. Pat. Nos. 6,214,790, 6,169,057, 5,776,970, 4,870,097, 4,576,750 and 4,299,838. Other Trp analogs include variants that are substituted (e.g., by a methyl group) at the α or β carbon and, optionally, also at one or more positions of the indole ring. Amino acids comprising two or more aromatic rings, including substituted, unsubstituted, or alternatively substituted variants thereof, are of interest as Trp analogs. In certain embodiments of the invention the Trp analog, e.g., at position 4, is 5-methoxy, 5-methyl-, 1-methyl-, or 1-formyl-tryptophan. In certain embodiments of the invention a Trp analog (e.g., at position 4) comprising a 1-alkyl substituent, e.g., a lower alkyl (e.g., C₁-C₅) substituent is used. In certain embodiments, N(α) methyl tryptophan or 5-methyltryptophan is used. In some embodiments, an analog comprising a 1-alkanyol substituent, e.g., a lower alkanoyl (e.g., C₁-C₅) is used. Examples include 1-acetyl-L-tryptophan and L-β-tryptophan.

In certain embodiments the Trp analog has increased hydrophobic character relative to Trp. For example, the indole ring may be substituted by one or more alkyl (e.g., methyl) groups. In certain embodiments the Trp analog participates in a hydrophobic interaction with C3. Such a Trp analog may be located, e.g., at position 4 relative to the sequence of compstatin. In certain embodiments the Trp analog comprises a substituted or unsubstituted bicyclic aromatic ring component or two or more substituted or unsubstituted monocyclic aromatic ring components.

In certain embodiments the Trp analog has increased propensity to form hydrogen bonds with C3 relative to Trp but does not have increased hydrophobic character relative to Trp. The Trp analog may have increased polarity relative to Trp and/or an increased ability to participate in an electrostatic interaction with a hydrogen bond donor on C3. Certain exemplary Trp analogs with an increased hydrogen bond forming character comprise an electronegative substituent on the indole ring. Such a Trp analog may be located, e.g., at position 7 relative to the sequence of compstatin.

In certain embodiments of the invention the compstatin analog comprises one or more Ala analogs (e.g., at position 9 relative to the sequence of compstatin), e.g., Ala analogs that are identical to Ala except that they include one or more CH₂ groups in the side chain. In certain embodiments the Ala analog is an unbranched single methyl amino acid such as 2-Abu. In certain embodiments of the invention the compstatin analog comprises one or more Trp analogs (e.g., at position 4 and/or 7 relative to the sequence of compstatin) and an Ala analog (e.g., at position 9 relative to the sequence of compstatin).

In certain embodiments of the invention the compstatin analog is a compound that comprises a peptide that has a sequence of (X′aa)ₙ-Gln-Asp-Xaa-Gly-(X″aa)ₘ, (SEQ ID NO: 2) wherein each X′aa and each X″aa is an independently selected amino acid or amino acid analog, wherein Xaa is Trp or an analog of Trp, and wherein n>1 and m>1 and n+m is between 5 and 21. The peptide has a core sequence of Gln-Asp-Xaa-Gly, where Xaa is Trp or an analog of Trp, e.g., an analog of Trp having increased propensity to form hydrogen bonds with an H-bond donor relative to Trp but, in certain embodiments, not having increased hydrophobic character relative to Trp. For example, the analog may be one in which the indole ring of Trp is substituted with an electronegative moiety, e.g., a halogen such as fluorine. In one embodiment Xaa is 5-fluorotryptophan. Absent evidence to the contrary, one of skill in the art would recognize that any non-naturally occurring peptide whose sequence comprises this core sequence and that inhibits complement activation and/or binds to C3 will have been designed based on the sequence of compstatin. In an alternative embodiment Xaa is an amino acid or amino acid analog other than a Trp analog that allows the Gln-Asp-Xaa-Gly peptide to form a β-turn.

In certain embodiments of the invention the peptide has a core sequence of X′aa-Gln-Asp-Xaa-Gly (SEQ ID NO: 3), where X′aa and Xaa are selected from Trp and analogs of Trp. In certain embodiments of the invention the peptide has a core sequence of X′aa-Gln-Asp-Xaa-Gly (SEQ ID NO: 3), where X′aa and Xaa are selected from Trp, analogs of Trp, and other amino acids or amino acid analogs comprising at least one aromatic ring. In certain embodiments of the invention the core sequence forms a β-turn in the context of the peptide. The β-turn may be flexible, allowing the peptide to assume two or more conformations as assessed for example, using nuclear magnetic resonance (NMR). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention X′aa is an analog of Trp that has increased hydrophobic character relative to Trp. For example, X′aa may be 1-methyltryptophan. In certain embodiments of the invention Xaa is an analog of Trp that has increased propensity to form hydrogen bonds relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan.

In certain embodiments of the invention the peptide has a core sequence of X′aa-Gln-Asp-Xaa-Gly-X″aa (SEQ ID NO: 4), where X′aa and Xaa are each independently selected from Trp and analogs of Trp and X″aa is selected from His, Ala, analogs of Ala, Phe, and Trp. In certain embodiments of the invention X′aa is an analog of Trp that has increased hydrophobic character relative to Trp, such as 1-methyltryptophan or another Trp analog having an alkyl substituent on the indole ring (e.g., at position 1, 4, 5, or 6). In certain embodiments X′aa is an analog of Trp that comprises a substituted or unsubstituted bicyclic aromatic ring component or two or more substituted or unsubstituted monocyclic aromatic ring components. In certain embodiments of the invention X′aa is selected from the group consisting of 2-napthylalanine, 1-napthylalanine, 2-indanylglycine carboxylic acid, dihydrotryptophan, and benzoylphenylalanine. In certain embodiments of the invention Xaa is an analog of Trp that has increased propensity to form hydrogen bonds with C3 relative to Trp but, in certain embodiments, not having increased hydrophobic character relative to Trp. In certain embodiments of the invention the analog of Trp that has increased propensity to form hydrogen bonds relative to Trp comprises a modification on the indole ring of Trp, e.g., at position 5, such as a substitution of a halogen atom for an H atom at position 5. For example, Xaa may be 5-fluorotryptophan. In certain embodiments X″aa is Ala or an analog of Ala such as Abu or another unbranched single methyl amino acid. In certain embodiments of the invention the peptide has a core sequence of X′aa-Gln-Asp-Xaa-Gly-X″aa (SEQ ID NO: 4), where X′aa and Xaa are each independently selected from Trp, analogs of Trp, and amino acids or amino acid analogs comprising at least one aromatic side chain, and X″aa is selected from His, Ala, analogs of Ala, Phe, and Trp. In certain embodiments X″aa is selected from analogs of Trp, aromatic amino acids, and aromatic amino acid analogs.

In certain preferred embodiments of the invention the peptide is cyclic. The peptide may be cyclized via a bond between any two amino acids, one of which is (X′aa)ₙ and the other of which is located within (X″aa)ₘ. In certain embodiments the cyclic portion of the peptide is between 9 and 15 amino acids in length, e.g., 10⁻¹² amino acids in length. In certain embodiments the cyclic portion of the peptide is 11 amino acids in length, with a bond (e.g., a disulfide bond) between amino acids at positions 2 and 12. For example, the peptide may be 13 amino acids long, with a bond between amino acids at positions 2 and 12 resulting in a cyclic portion 11 amino acids in length.

In certain embodiments the peptide comprises or consists of the sequence X′aa1-X′aa2-X′aa3-X′aa4-Gln-Asp-Xaa-Gly-X″aa1-X″aa2-X″aa3-X″aa4-X″aa5 (SEQ ID NO: 5). In certain embodiments X′aa4 and Xaa are selected from Trp and analogs of Trp, and X′aa1, X′aa2, X′aa3, X″aa1, X″aa2, X″aa3, X″aa4, and X″aa5 are independently selected from among amino acids and amino acid analogs. In certain embodiments X′aa4 and Xaa are selected from aromatic amino acids and aromatic amino acid analogs. Any one or more of X′aa1, X′aa2, X′aa3, X″aa1, X″aa2, X″aa3, X″aa4, and X″aa5 may be identical to the amino acid at the corresponding position in compstatin. In one embodiment, X″aa1 is Ala or a single methyl unbranched amino acid. The peptide may be cyclized via a covalent bond between (i) X′aa1, X′aa2, or X′aa3; and (ii) X″aa2, X″aa3, X″aa4 or X″aa5. In one embodiment the peptide is cyclized via a covalent bond between X′aa2 and X″aa4. In one embodiment the covalently bound amino acid are each Cys and the covalent bond is a disulfide (S—S) bond. In some embodiments the covalent bond is a C—C, C—O, C—S, or C—N bond. In certain embodiments one of the covalently bound residues is an amino acid or amino acid analog having a side chain that comprises a primary or secondary amine, the other covalently bound residue is an amino acid or amino acid analog having a side chain that comprises a carboxylic acid group, and the covalent bond is an amide bond. Amino acids or amino acid analogs having a side chain that comprises a primary or secondary amine include lysine and diaminocarboxylic acids of general structure NH₂(CH₂)ₙCH(NH₂)COOH such as 2,3-diaminopropionic acid (dapa), 2,4-diaminobutyric acid (daba), and omithine (om), wherein n=1 (dapa), 2 (daba), and 3 (om), respectively. Examples of amino acids having a side chain that comprises a carboxylic acid group include dicarboxylic amino acids such as glutamic acid and aspartic acid. Analogs such as beta-hydroxy-L-glutamic acid may also be used. In some embodiments a peptide is cyclized with a thioether bond, e.g., as described in PCT/US2011/052442 (WO/2012/040259) or US Pat. App. Pub. No. 20140113874. For example, in some embodiments a disulfide bond in any of the peptides is replaced with a thioether bond. In some embodiments, a cystathionine is formed. In some embodiments the cystathionine is a delta-cystathionine or a gamma-cystathionine. In some embodiments a modification comprises replacement of a Cys-Cys disulfide bond between cysteines at X′aa2 and X″aa4 in SEQ ID NO: 5 (or corresponding positions in other sequences) with addition of a CH₂, to form a homocysteine at X′aa2 or X″aa4, and introduction of a thioether bond, to form a cystathionine. In one embodiment, the cystathionine is a gamma-cystathionine. In some embodiments, the cystathionine is a delta-cystathionine. Another modification of use in certain embodiments comprises replacement of the disulfide bond with a thioether bond without the addition of a CH₂, thereby forming a lantithionine. In some embodiments a compstatin analog having a thioether in place of a disulfide bond has increased stability, at least under some conditions, as compared with the compstatin analog having the disulfide bond.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 6); wherein:

Xaa1 is Ile, Val, Leu, B¹-Ile, B¹-Val, B¹-Leu or a dipeptide comprising Gly-Ile or B¹-Gly-Ile, and B¹ represents a first blocking moiety;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of the L-Thr, D-Thr, Ile, Val, Gly, Ala, or Asn optionally is replaced by a second blocking moiety B²; and the two Cys residues are joined by a disulfide bond. In some embodiments, Xaa4 is Leu, Nle, His, or Phe or a depeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B²; and the two Cys residues are joined by a disulfide bond.

In some embodiments Xaa1 is absent or is any amino acid or amino acid analog, and Xaa2, Xaa2*, Xaa3, and Xaa4 are as defined above. If Xaa1 is absent, the N-terminal Cys residue may have a blocking moiety B¹ attached thereto.

In some embodiments, Xaa4 is any amino acid or amino acid analog and Xaa1, Xaa2, Xaa2*, and Xaa3 are as defined above. In some embodiments, Xaa4 is a dipeptide selected from the group consisting of: Thr-Ala and Thr-Asn, wherein the carboxy terminal —OH or the Ala or Asn is optionally replaced by a second blocking moiety B².

In any of the embodiments of the compstatin analog of SEQ ID NO: 6, Xaa2 may be Trp.

In any of the embodiments of the compstatin analog of SEQ ID NO: 6, Xaa2 may be an analog of Trp comprising a substituted or unsubstituted bicyclic aromatic ring component or two or more substituted or unsubstituted monocyclic aromatic ring components. For example, the analog of Trp may be selected from 2-naphthylalanine (2-NaI), 1-naphthylalanine (1-NaI), 2-indanylglycine carboxylic acid (Ig1), dihydrotrpytophan (Dht), and 4-benzoyl-L-phenylalanine.

In any of the embodiments of the compstatin analog of SEQ ID NO: 6, Xaa2 may be an analog of Trp having increased hydrophobic character relative to Trp. For example, the analog of Trp may be selected from 1-methyltryptophan, 4-methyltryptophan, 5-methyltryptophan, and 6-methyltryptophan. In one embodiment, the analog of Trp is 1-methyltryptophan. In one embodiment, Xaa2 is 1-methyltryptophan, Xaa2* is Trp, Xaa3 is Ala, and the other amino acids are identical to those of compstatin.

In any of the embodiments of the compstatin analog of SEQ ID NO: 6, Xaa2* may be an analog of Trp such as an analog of Trp having increased hydrogen bond forming propensity with C3 relative to Trp, which, in certain embodiments, does not have increased hydrophobic character relative to Trp. In certain embodiments the analog of Trp comprises an electronegative substituent on the indole ring. For example, the analog of Trp may be selected from 5-fluorotryptophan and 6-fluorotryptophan.

In certain embodiments of the invention Xaa2 is Trp and Xaa2* is an analog of Trp having increased hydrogen bond forming propensity with C3 relative to Trp which, in certain embodiments, does not have increased hydrophobic character relative to Trp. In certain embodiments of the compstatin analog of SEQ ID NO: 6, Xaa2 is analog of Trp having increased hydrophobic character relative to Trp such as an analog of Trp selected from 1-methyltryptophan, 4-methyltryptophan, 5-methyltryptophan, and 6-methyltryptophan, and and Xaa2* is an analog of Trp having increased hydrogen bond forming propensity with C3 relative to Trp which, in certain embodiments, does not have increased hydrophobic character relative to Trp. For example, in one embodiment Xaa2 is methyltryptophan and Xaa2* is 5-fluorotryptophan.

In certain of the afore-mentioned embodiments, Xaa3 is Ala. In certain of the afore-mentioned embodiments Xaa3 is a single methyl unbranched amino acid, e.g., Abu.

The invention further provides compstatin analogs of SEQ ID NO: 6, as described above, wherein Xaa2 and Xaa2* are independently selected from Trp, analogs of Trp, and other amino acids or amino acid analogs that comprise at least one aromatic ring, and Xaa3 is His, Ala or an analog of Ala, Phe, Trp, an analog of Trp, or another aromatic amino acid or aromatic amino acid analog.

In certain embodiments of the invention the blocking moiety present at the N- or C-terminus of any of the compstatin analogs described herein is any moiety that stabilizes a peptide against degradation that would otherwise occur in mammalian (e.g., human or non-human primate) blood or interstitial fluid. For example, blocking moiety B could be any moiety that alters the structure of the N-terminus of a peptide so as to inhibit cleavage of a peptide bond between the N-terminal amino acid of the peptide and the adjacent amino acid. Blocking moiety B² could be any moiety that alters the structure of the C-terminus of a peptide so as to inhibit cleavage of a peptide bond between the C-terminal amino acid of the peptide and the adjacent amino acid. Any suitable blocking moieties known in the art could be used. In certain embodiments of the invention blocking moiety B¹ comprises an acyl group (i.e., the portion of a carboxylic acid that remains following removal of the —OH group). The acyl group typically comprises between 1 and 12 carbons, e.g., between 1 and 6 carbons. For example, in certain embodiments of the invention blocking moiety B¹ is selected from the group consisting of: formyl, acetyl, proprionyl, butyryl, isobutyryl, valeryl, isovaleryl, etc. In one embodiment, the blocking moiety B is an acetyl group, i.e., Xaa1 is Ac-Ile, Ac-Val, Ac-Leu, or Ac-Gly-Ile.

In certain embodiments of the invention blocking moiety B² is a primary or secondary amine (—NH₂ or —NHR¹, wherein R is an organic moiety such as an alkyl group).

In certain embodiments of the invention blocking moiety B¹ is any moiety that neutralizes or reduces the positive charge that may otherwise be present at the N-terminus at physiological pH. In certain embodiments of the invention blocking moiety B² is any moiety that neutralizes or reduces the negative charge that may otherwise be present at the C-terminus at physiological pH.

In certain embodiments of the invention, the compstatin analog is acetylated or amidated at the N-terminus and/or C-terminus, respectively. A compstatin analog may be acetylated at the N-terminus, amidated at the C-terminus, and or both acetylated at the N-terminus and amidated at the C-terminus. In certain embodiments of the invention a compstatin analog comprises an alkyl or aryl group at the N-terminus rather than an acetyl group.

In certain embodiments, the compstatin analog is a compound that comprises a peptide having a sequence:

Xaa1-Cys-Val-Xaa2-Gln-Asp-Xaa2*-Gly-Xaa3-His-Arg-Cys-Xaa4 (SEQ ID NO: 7); wherein:

Xaa1 is Ile, Val, Leu, Ac-Ile, Ac-Val, Ac-Leu or a dipeptide comprising Gly-Ile or Ac-Gly-Ile;
Xaa2 and Xaa2* are independently selected from Trp and analogs of Trp;
Xaa3 is His, Ala or an analog of Ala, Phe, Trp, or an analog of Trp;
Xaa4 is L-Thr, D-Thr, Ile, Val, Gly, a dipeptide selected from Thr-Ala and Thr-Asn, or a tripeptide comprising Thr-Ala-Asn, wherein a carboxy terminal —OH of any of L-Thr, D-Thr, Ile, Val, Gly, Ala, or Asn optionally is replaced by —NH₂; and the two Cys residues are joined by a disulfide bond. In some embodiments, Xaa4 is Leu, Nle, His, or Phe or a depeptide selected from Xaa5-Ala and Xaa5-Asn, or a tripeptide Xaa5-Ala-Asn, wherein Xaa5 is selected from Leu, Nle, His or Phe, and wherein a carboxy terminal —OH of any of the L-Thr, D-Thr, Ile, Val, Gly, Leu, Nle, His, Phe, Ala, or Asn optionally is replaced by a second blocking moiety B2; and the two Cys residues are joined by a disulfide bond.

In some embodiments, Xaa1, Xaa2, Xaa2*, Xaa3, and Xaa4 are as described above for the various embodiments of SEQ ID NO: 6. For example, in certain embodiments Xaa2* is Trp. In certain embodiments Xaa2 is an analog of Trp having increased hydrophobic character relative to Trp, e.g., 1-methyltryptophan. In certain embodiments Xaa3 is Ala. In certain embodiments Xaa3 is a single methyl unbranched amino acid.

In certain embodiments of the invention Xaa1 is Ile and Xaa4 is L-Thr.

In certain embodiments of the invention Xaa1 is Ile, Xaa2* is Trp, and Xaa4 is L-Thr.

The invention further provides compstatin analogs of SEQ ID NO: 7, as described above, wherein Xaa2 and Xaa2* are independently selected from Trp, analogs of Trp, other amino acids or aromatic amino acid analogs, and Xaa3 is His, Ala or an analog of Ala, Phe, Trp, an analog of Trp, or another aromatic amino acid or aromatic amino acid analog.

In certain embodiments of any of the compstatin analogs described herein, an analog of Phe is used rather than Phe.

Table 1 provides a non-limiting list of compstatin analogs useful in the present invention. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 1 are amidated at the C-terminus. Bold text is used to indicate certain modifications. Activity relative to compstatin is based on published data and assays described therein (WO2004/026328, WO2007044668, Mallik, 2005; Katragadda, 2006). Where multiple publications reporting an activity were consulted, the more recently published value is used, and it will be recognized that values may be adjusted in the case of differences between assays. It will also be appreciated that in certain embodiments of the invention the peptides listed in Table 1 are cyclized via a disulfide bond between the two Cys residues when used in the therapeutic compositions and methods of the invention. Alternate means for cyclizing the peptides are also within the scope of the invention. As noted above, in various embodiments of the invention one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments of the invention, for example, a compstatin analog comprises an N-methyl glycine, e.g., at the position corresponding to position 8 of compstatin and/or at the position corresponding to position 13 of compstatin. In some embodiments, one or more of the compstatin analogs in Table 1 contains at least one N-methyl glycine, e.g., at the position corresponding to position 8 of compstatin and/or at the position corresponding to position 13 of compstatin. In some embodiments, one or more of the compstatin analogs in contains at least one N-methyl isoleucine, e.g., at the position corresponding to position 13 of compstatin. For example, a Thr at or near the C-terminal end of a peptide whose sequence is listed in Table 1 may be replaced by N-methyl Ile. As will be appreciated, in some embodiments the N-methylated amino acids comprise N-methyl Gly at position 8 and N-methyl Ile at position 13. In some embodiments the N-methylated amino acids comprise N-methyl Gly in a core sequence such as SEQ ID NO: 3 or SEQ ID NO: 4.

TABLE 1

| | | SEQ ID | Activity over |
|---|---|---|---|
| Peptide | Sequence | NO: | compstatin |
| Compstatin | _{H-}ICVVQDWGHHRCT_{-CONH2} | 8 | * |
| Ac-compstatin | _{Ac-}ICVVQDWGHHRCT_{-CONH2} | 9 | 3× more |
| Ac-V4Y/H9A | _{Ac-}ICVYQDWGAHRCT_{-CONH2} | 10 | 14× more |
| Ac-V4W/H9A -OH | _{Ac-}ICVWQDWGAHRCT_{-COOH} | 11 | 27× more |
| Ac-V4W/H9A | _{Ac-}ICVWQDWGAHRCT_{-CONH2} | 12 | 45× more |
| Ac-V4W/H9A/T13dT -OH | _{Ac-}ICVWQDWGAHRCdT_{-COOH} | 13 | 55× more |
| Ac-V4(2-Nal)/H9A | _{Ac-}ICV(2-Nal)QDWGAHRCT_{-CONH2} | 14 | 99× more |
| Ac-V4(2-Nal)/H9A -OH | _{Ac-}ICV(2-Nal)QDWGAHRCT_{-COOH} | 15 | 38× more |
| Ac-V4(1-Nal)/H9A -OH | _{Ac-}ICV(1-Nal)QDWGAHRCT_{-COOH} | 16 | 30× more |
| Ac-V42Igl/H9A | _{Ac-}ICV(2-Igl)QDWGAHRCT_{-CONH2} | 17 | 39× more |
| Ac-V42Igl/H9A -OH | _{Ac-}ICV(2-Igl)QDWGAHRCT_{-COOH} | 18 | 37× more |
| Ac-V4Dht/H9A -OH | _{Ac-}ICVDhtQDWGAHRCT_{-COOH} | 19 | 5× more |
| Ac-V4(Bpa)/H9A -OH | _{Ac-}ICV(Bpa)QDWGAHRCT_{-COOH} | 20 | 49× more |
| Ac-V4(Bpa)/H9A | _{Ac-}ICV(Bpa)QDWGAHRCT_{-CONH2} | 21 | 86× more |
| Ac-V4(Bta)/H9A -OH | _{Ac-}ICV(Bta)QDWGAHRCT_{-COOH} | 22 | 65× more |
| Ac-V4(Bta)/H9A | _{Ac-}ICV(Bta)QDWGAHRCT_{-CONH2} | 23 | 64× more |
| Ac-V4W/H9(2-Abu) | _{Ac-}ICVWQDWG(2-Abu)HRCT_{-CONH2} | 24 | 64× more |
| +G/V4W/H9A +AN -OH | _{H}-GICVWQDWGAHRCTAN_{-COOH} | 25 | 38× more |
| Ac-V4(5fW)/H9A | _{Ac-}ICV(5fW)QDWGAHRCT_{-CONH2} | 26 | 31× more |
| Ac-V4(5-MeW)/H9A | _{Ac-}ICV(5-methyl-W)QDWGAHRCT_{-CONH2} | 27 | 67× more |
| Ac-V4(1-MeW)/H9A | _{Ac-}ICV(1-methyl-W)QDWGAHRCT_{-CONH2} | 28 | 264× more |
| Ac-V4W/W7(5fW)/H9A | _{Ac-}ICVWQD(5fW)GAHRCT_{-CONH2} | 29 | 121× more |
| Ac-V4(5fW)/W7(5fW)/H9A | _{Ac-}ICV(5fW)QD(5fW)GAHRCT_{-CONH2} | 30 | NA |
| Ac-V4(5-MeW)/W7(5fW)H9A | _{Ac-}ICV(5-methyl-W)QD(5fW)GAHRCT_{-CONH2} | 31 | NA |
| Ac-V4(1MeW)/W7(5fW)/H9A | _{Ac-}ICV(1-methyl-W)QD(5fW)GAHRCT_{-CONH2} | 32 | 264× more |
| +G/V4(6fW)/W7(6fW)H9A+N-OH | _{H-}GICV(6fW)QD(6fW)GAHRCTN_{-COOH} | 33 | 126× more |
| Ac-V4(1-formyl-W)/H9A | _{Ac-}ICV(1-formyl-W)QDWGAHRCT_{-CONH2} | 34 | 264× more |
| Ac-V4(5-methoxy-W)/H9A | _{Ac-}ICV(1-methoxy-W)QDWGAHRCT_{-CONH2} | 35 | 76× more |
| G/V4(5f-W)/W7(5fW)/H9A+N-OH | _{H-}GICV(5fW)QD(5fW)GAHRCTN_{-COOH} | 36 | 112× more |

NA = not available

In certain embodiments of the compositions and methods of the invention the compstatin analog has a sequence selected from sequences 9-36. In certain embodiments of the compositions and methods of the invention the compstatin analog has a sequence selected from SEQ ID NOs: 14, 21, 28, 29, 32, 33, 34, and 36. In certain embodiments of the compositions and/or methods of the invention the compstatin analog has a sequence selected from SEQ ID NOs: 30 and 31. In one embodiment of the compositions and methods of the invention the compstatin analog has a sequence of SEQ ID NO: 28. In one embodiment of the compositions and methods of the invention the compstatin analog has a sequence of SEQ ID NO: 32. In one embodiment of the compositions and methods of the invention the compstatin analog has a sequence of SEQ ID NO: 34. In one embodiment of the compositions and methods of the invention the compstatin analog has a sequence of SEQ ID NO: 36.

In some embodiments a blocking moiety B¹ comprises an amino acid, which may be represented as Xaa0. In some embodiments blocking moiety B2 comprises an amino acid, which may be represented as XaaN. In some embodiments blocking moiety B¹ and/or B² comprises a non-standard amino acid, such as a D-amino acid, N-alkyl amino acid (e.g., N-methyl amino acid). In some embodiments a blocking moiety B and/or B² comprises a non-standard amino acid that is an analog of a standard amino acid. In some embodiments an amino acid nalog comprises a lower alkyl, lower alkoxy, or halogen substituent, as compared with a standard amino acid of which it is an analog. In some embodiments a substituent is on a side chain. In some embodiments a substituent is on an alpha carbon atom. In some embodiments, a blocking moiety B comprising an amino acid, e.g., a non-standard amino acid, further comprises a moiety B^{1a}. For example, blocking moiety B¹ may be represented as B^{1a}-Xaa0. In some embodiments B^{1a} neutralizes or reduces a positive charge that may otherwise be present at the N-terminus at physiological pH. In some embodiments B^{1a} comprises or consists of, e.g., an acyl group that, e.g., comprises between 1 and 12 carbons, e.g., between 1 and 6 carbons. In certain embodiments blocking moiety B^{1a} is selected from the group consisting of: formyl, acetyl, proprionyl, butyryl, isobutyryl, valeryl, isovaleryl, etc. In some embodiments, a blocking moiety B² comprising an amino acid, e.g., a non-standard amino acid, may further comprise a moiety B^{2a} For example, blocking moiety B² may be represented as XaaN-B^{2a}, where N represents the appropriate number for the amino acid (which will depend on the numbering used in the rest of the peptide). In some embodiments B^{2a} neutralizes or reduces a negative charge that may otherwise be present at the C-terminus at physiological pH. In some embodiments B^{2a} comprises or consists of a primary or secondary amine (e.g., NH₂). It will be understood that a blocking activity of moiety B^{1a}-Xaa0 and/or XaaN-B^{2a} may be provided by either or both components of the moiety in various embodiments. In some embodiments a blocking moiety or portion thereof, e.g., an amino acid residue, may contribute to increasing affinity of the compound for C3 or C3b and/or improve the activity of the compound. In some embodiments a contribution to affinity or activity of an amino acid residue may be at least as important as a contribution to blocking activity. For example, in some embodiments Xaa0 and/or XaaN in B^{1a}-Xaa0 and/or XaaN-B^{2a} may function mainly to increase affinity or activity of the compound, while B^{1a} and/or B^{2a} may inhibit digestion of and/or neutralize a charge of the peptide. In some embodiments a compstatin analog comprises the amino acid sequence of any of SEQ ID NOs: 5-36, wherein SEQ ID NOs: 5-36 is further extended at the N- and/or C-terminus. In some embodiments, the sequence may be represented as B^{1a}-Xaa0-SEQUENCE-XaaN-B^{2a} where SEQUENCE represents any of SEQ ID NOs: 5-36, wherein B^{1a} and B^{2a} may independently be present or absent. For example, in some embodiments a compstatin analog comprises B^{1a}-Xaa0-X′aa1-X′aa2-X′aa3-X′aa4-Gln-Asp-Xaa-Gly-X″aa1-X″aa2-X″aa3-X″aa4-X″aa5-XaaN-B^{2a} (SEQ ID NO: 37), where X′aa1-X′aa2-X′aa3-X′aa4, Xaa, X″aa1, X″aa2, X″aa3, X″aa4, and X″aa5 are as set forth above for SEQ ID NO: 5.

In some embodiments a compstatin analog comprises B^{1a}-Xaa0-Xaa1-Cys-Val-Xaa2-Gln-Asp-Xaa2*-Gly-Xaa3-His-Arg-Cys-Xaa4-XaaN-B^{2a} (SEQ ID NO: 38), where Xaa1, Xaa2, Xaa2*, Xaa3, and Xaa4 are as set forth above for SEQ ID NO: 6 or wherein Xaa1, Xaa2, Xaa2*, Xaa3, and Xaa4 are as set forth for SEQ ID NO: 6 or SEQ ID NO: 7.

In some embodiments a compstatin analog comprises B^{1a}-Xaa0-Xaa1-Xaa2-Xaa3-Xaa4-Xaa5-Xaa6-Xaa7-Xaa8-Xaa9-Xaa10-Xaa11-Xaa12-Xaa13-XaaN-B^{2a} (SEQ ID NO: 39) wherein Xaa1, Xaa2, Xaa3, Xaa4, Xaa5, Xaa6, Xaa7, Xaa8, Xaa9, Xaa10, Xaa11, Xaa12, and Xaa13 are identical to amino acids at positions 1-13 of any of SEQ ID NOs: 9-36.

In some embodiments Xaa0 and/or XaaN in any compstatin analog sequence comprises an amino acid that comprises an aromatic ring having an alkyl substituent at one or more positions. In some embodiments an alkyl substituent is a lower alkyl substituent. For example, in some embodiments an alkyl substituent is a methyl or ethyl group. In some embodiments a substituent is located at any position that does not destroy the aromatic character of the compound. In some embodiments a substituent is located at any position that does not destroy the aromatic character of a ring to which the substituent is attached. In some embodiments a substituent is located at position 1, 2, 3, 4, or 5. In some embodiments Xaa0 comprises an O-methyl analog of tyrosine, 2-hydroxyphenylalanine or 3-hydroxyphenylalanine. For purposes of the present disclosure, a lower case “m” followed by a three letter amino acid abbreviation may be used to specifically indicate that the amino acid is an N-methyl amino acid. For example, where the abbreviation “mGly” appears herein, it denotes N-methyl glycine (also sometimes referred to as sarcosine or Sar). In some embodiments Xaa0 is or comprises mGly, Tyr, Phe, Arg, Trp, Thr, Tyr(Me), Cha, mPhe, mVal, mIle, mAla, DTyr, DPhe, DArg, DTrp, DThr, DTyr(Me), mPhe, mVal, mIle, DAla, or DCha. For example, in some embodiments a compstatin analog comprises a peptide having a sequence B¹-Ile-[Cys-Val-Trp(Me)-Gln-Asp-Trp-mGly-Ala-His-Arg-Cys]-mIle-B² (SEQ ID NO: 40) or B¹-Ile-[Cys-Val-Trp(Me)-Gln-Asp-Trp-mGly-Ala-His-Arg-Cys]-mIle-B² (SEQ ID NO: 41). The two Cys residues are joined by a disulfide bond in the active compounds. In some embodiments the peptide is acetylated at the N-terminus and/or amidated at the C-terminus. In some embodiments B¹ comprises B^{1a}-Xaa0 and/or B2 comprises XaaN-B2a, as described above. For example, in some embodiments B comprises or consists of Gly, mGly, Tyr, Phe, Arg, Trp, Thr, Tyr(Me), mPhe, mVal, mIle, mAla, DTyr, DPhe, DTrp, DCha, DAla and B² comprises NH₂, e.g., a carboxy terminal —OH of mIle is replaced by NH₂. In some embodiments B¹ comprises or consists of mGly, Tyr, DTyr, or Tyr(Me) and B² comprises NH₂, e.g., a carboxy terminal —OH of mIle is replaced by NH₂. In some embodiments an Ile at position Xaa1 is replaced by Gly. Complement inhibition potency and/or C3b binding parameters of selected compstatin analogs are described in WO/2010/127336 (PCT/US2010/033345), US Pat. App. Pub. No. 20120178694 and/or in Qu, et al., Immunobiology (2012), doi:10.1016/j.imbio.2012.06.003.

In some embodiments a blocking moiety or portion thereof, e.g., an amino acid residue, may contribute to increasing affinity of the compound for C3 or C3b and/or improve the activity of the compound. In some embodiments a contribution to affinity or activity of an amino acid or amino acid analog may be more significant than a blocking activity.

In certain embodiments of the compositions and methods of the invention the compstatin analog has a sequence as set forth in Table 1, but where the Ac-group is replaced by an alternate blocking moiety B¹, as described herein. In some embodiments the —NH₂ group is replaced by an alternate blocking moiety B², as described herein.

In one embodiment, the compstatin analog binds to substantially the same region of the β chain of human C3 as does compstatin. In one embodiment the compstatin analog is a compound that binds to a fragment of the C-terminal portion of the β chain of human C3 having a molecular weight of about 40 kDa to which compstatin binds (Soulika, A. M., et al., Mol. Immunol., 35:160, 1998; Soulika, A. M., et al., Mol. Immunol. 43(12):2023-9, 2006). In certain embodiments the compstatin analog is a compound that binds to the binding site of compstatin as determined in a compstatin-C3 structure, e.g., a crystal structure or NMR-derived 3D structure. In certain embodiments the compstatin analog is a compound that could substitute for compstatin in a compstatin-C3 structure and would form substantially the same intermolecular contacts with C3 as compstatin. In certain embodiments the compstatin analog is a compound that binds to the binding site of a peptide having a sequence set forth in Table 1, e.g., SEQ ID NO: 14, 21, 28, 29, 32, 33, 34, or 36 or another compstatin analog sequence disclosed herein in a peptide-C3 structure, e.g., a crystal structure. In certain embodiments the compstatin analog is a compound that binds to the binding site of a peptide having SEQ ID NO: 30 or 31 in a peptide-C3 structure, e.g., a crystal structure. In certain embodiments the compstatin analog is a compound that could substitute for the peptide of SEQ ID NO: 9-36, e.g., a compound that could substitute for the peptide of SEQ ID NO: 14, 21, 28, 29, 32, 33, 34, or 36 or another compstatin analog sequence disclosed herein in a peptide-C3 structure and would form substantially the same intermolecular contacts with C3 as the peptide. In certain embodiments the compstatin analog is a compound that could substitute for the peptide of SEQ ID NO: 30 or 31 in a peptide-C3 structure and would form substantially the same intermolecular contacts with C3 as the peptide.

One of ordinary skill in the art will readily be able to determine whether a compstatin analog binds to a fragment of the C-terminal portion of the β chain of C3 using routine experimental methods. For example, one of skill in the art could synthesize a photocrosslinkable version of the compstatin analog by including a photo-crosslinking amino acid such as p-benzoyl-L-phenylalanine (Bpa) in the compound, e.g., at the C-terminus of the sequence (Soulika, A. M., et al, supra). Optionally additional amino acids, e.g., an epitope tag such as a FLAG tag or an HA tag could be included to facilitate detection of the compound, e.g., by Western blotting. The compstatin analog is incubated with the fragment and crosslinking is initiated. Colocalization of the compstatin analog and the C3 fragment indicates binding. Surface plasmon resonance may also be used to determine whether a compstatin analog binds to the compstatin binding site on C3 or a fragment thereof. One of skill in the art would be able to use molecular modeling software programs to predict whether a compound would form substantially the same intermolecular contacts with C3 as would compstatin or a peptide having the sequence of any of the peptides in Table 1, e.g., SEQ ID NO: 14, 21, 28, 29, 32, 33, 34, or 36, or in some embodiments SEQ ID NO: 30 or 31 or another compstatin analog sequence disclosed herein.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or W2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3^{rd} ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) or similar molecules to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S. W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, Calif., which also provides details of appropriate conjugation procedures. In some embodiments, a compstatin analog is fused to the Fc domain of an immunoglobulin or a portion thereof. In some embodiments a compstatin analog is conjugated to an albumin moiety or to an albumin binding moiety, e.g., an albumin binding peptide or small molecule. Thus in some embodiments a compstatin analog is modified with one or more polypeptide or non-polypeptide components, e.g., the compstatin analog is pegylated or conjugated to another moiety. In some embodiments the component is not the Fc domain of an immunoglobulin or a portion thereof. A compstatin analog can be provided as a multimer or as part of a supramolecular complex, which can include either a single molecular species or multiple different species (e.g., multiple different analogs).

In some embodiments, a compstatin analog is a multivalent compound comprising a plurality of compstatin analog moieties covalently or noncovalently linked to a polymeric backbone or scaffold. The compstatin analog moieties can be identical or different. In certain embodiments of the invention the multivalent compound comprises multiple instances, or copies, of a single compstatin analog moiety. In some embodiments of the disclosure the multivalent compound comprises one or more instances of each of two of more non-identical compstatin analog moieties, e.g., 3, 4, 5, or more different compstatin analog moieties. In certain embodiments of the invention the number of compstatin analog moieties (“n”) is between 2 and 6. In some embodiments of the invention n is between 7 and 20. In some embodiments of the invention n is between 20 and 100. In some embodiments n is between 100 and 1,000. In some embodiments of the invention n is between 1,000 and 10,000. In some embodiments n is between 10,000 and 50,000. In some embodiments n is between 50,000 and 100,000. In some embodiments n is between 100,000 and 1,000,000.

The compstatin analog moieties may be attached directly to the polymeric scaffold or may be attached via a linking moiety that connects the compstatin analog moiety to the polymeric scaffold. The linking moiety may be attached to a single compstatin analog moiety and to the polymeric scaffold. Alternately, a linking moiety may have multiple compstatin analog moieties joined thereto so that the linking moiety attaches multiple compstatin analog moieties to the polymeric scaffold.

In some embodiments, a compstatin analog comprises an amino acid having a side chain comprising a primary or secondary amine, e.g., a Lys residue. For example, any of the compstatin analog sequences disclosed herein may be extended or modified by addition of a linker comprising one or more amino acids, e.g., one or more amino acids comprising a primary or secondary amine, e.g., in a side chain thereof. For example, a Lys residue, or a sequence comprising a Lys residue, is added at the N-terminus and/or C-terminus of the compstatin analog. In some embodiments, the Lys residue is separated from the cyclic portion of the compstatin analog by a rigid or flexible spacer. A linker or spacer may, for example, comprise a substituted or unsubstituted, saturated or unsaturated alkyl chain, oligo(ethylene glycol) chain, and/or other moieties. The length of the chain may be, e.g., between 2 and 20 carbon atoms. In some embodiments the spacer is or comprises a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. A PEG moiety or similar molecule or polymeric scaffold may be linked to the primary or secondary amine, optionally via a linker. In some embodiments, a bifunctional linker is used. A bifunctional linker may comprise two reactive functional groups, which may be the same or different in various embodiments. In various embodiments, one or more linkers, spacers, and/or techniques of conjugation described in Hermanson, supra, is used.

Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In some embodiments, short chains are prepolymerized, functionalized, and then a mixture of short chains of different composition are assembled into longer polymers.

In some aspects a moiety such as a polyethylene glycol (PEG) chain, polyoxazoline (POZ) chain, or other polymer(s), e.g., polypeptides, that, e.g., stabilize the compound, increase its lifetime in the body, increase its solubility, decrease its immunogenicity, and/or increase its resistance to degradation may be referred to herein as a “clearance reducing moiety” (CRM), and a compstatin analog comprising such a moiety may be referred to as a long-acting compstatin analog. In some embodiments a CRM comprises a non-polypeptide polymer or polypeptide that has a molecular weight of between 5 kD and 150 kD, e.g., at least 5, 10, 20, 30, 40, 50, 60, 70, 80, 90, 100 kD, or more, e.g., between 100 and 120, or 120 and 150 kD. In some embodiments the polymer is linear. In some embodiments the polymer is branched.

In some aspects, a long-acting compstatin analog comprises a compound of formula M-L-A, wherein A is a moiety that comprises a CRM, L is an optionally present linking portion, and M comprises a compstatin analog moiety. The compstatin analog moiety can comprise any compstatin analog, e.g., any compstatin analog described above, in various embodiments. Formula M-L-A encompasses embodiments in which L-A is present at the N-terminus of the compstatin analog moiety, embodiments in which L-A is present at the C-terminus of the compstatin analog moiety, embodiments in which L-A is attached to a side chain of an amino acid of the compstatin analog moiety, and embodiments where the same or different L-As are present at both ends of M. In some embodiments the same or different M (or M-L) may be present at the ends of a linear CRM or at the termini of branches of a branched CRM. It will be appreciated that when certain compstatin analog(s) are present as a compstatin analog moiety in a compound of formula M-L-A, a functional group of the compstatin analog will have reacted with a functional group of L to form a covalent bond to A or L. For example, a long-acting compstatin analog in which the compstatin analog moiety comprises a compstatin analog that contains an amino acid with a side chain containing a primary amine (NH₂) group (which compstatin analog can be represented by formula R¹—(NH₂)), can have a formula R¹—NH-L-A in which a new covalent bond to L (e.g., N—C) has been formed and a hydrogen lost. Thus the term “compstatin analog moiety” includes molecular structures in which at least one atom of a compstatin analog participates in a covalent bond with a second moiety, which may, e.g., modification of a side chain. Similar considerations apply to compstatin analog moieties present in multivalent compounds. In some embodiments, a blocking moiety at the N-terminus or C-terminus of a compstatin analog is replaced by L-A in the structure of a long-acting compstatin analog.

In some embodiments, L comprises an unsaturated moiety such as —CH═CH— or —CH₂—CH═CH—; a moiety comprising a non-aromatic cyclic ring system (e.g., a cyclohexyl moiety), an aromatic moiety (e.g., an aromatic cyclic ring system such as a phenyl moiety); an ether moiety (—C—O—C—); an amide moiety (—C(═O)—N—); an ester moiety (—CO—O—); a carbonyl moiety (—C(═O)—); an imine moiety (—C═N—); a thioether moiety (—C—S—C—); an amino acid residue; and/or any moiety that can be formed by the reaction of two compatible reactive functional groups. In certain embodiments, one or more moieties of a linking portion is/are substituted by independent replacement of one or more of the hydrogen (or other) atoms thereon with one or more moieties including, but not limited to aliphatic; aromatic, aryl; alkyl, aralkyl, alkanoyl, aroyl, alkoxy; thio; F; Cl; Br; I; —NO2; —CN; —CF3; —CH2CF3; —CHCl2; —CH2OH; —CH2CH2OH; —CH2NH2; —CH2SO2CH3; or —NGRG1 wherein G is —O—, —S—, —NRG2—, —C(═O)—, —S(═O)—, —SO2-, —C(═O)O—, —C(═O)NRG2-, —OC(═O)—, —NRG2C(═O)—, —OC(═O)O—, —OC(═O)NRG2-, —NRG2C(═O)O—, —NRG2C(═O)NRG2-, —C(═S)—, —C(═S)S—, —SC(═S)—, —SC(═S)S—, —C(═NRG2)-, —C(═NRG2)O—, —C(═NRG2)NRG3-, —OC(═NRG2)-, —NRG2C(═NRG3)-, —NRG2SO2-, —NRG2SO2NRG3-, or —SO2NRG2-, wherein each occurrence of RG1, RG2 and RG3 independently includes, but is not limited to, hydrogen, halogen, or an optionally substituted aliphatic, aromatic, or aryl moiety. It will be appreciated that cyclic ring systems when present as substituents may optionally be attached via a linear moiety. Combinations of substituents and variables are preferably those that result in the formation of stable compounds useful in any one or more of the methods described herein.

In some embodiments, a compstatin analog of use in compositions and methods described herein is a long-acting compstatin analog that has a terminal half-life of at least 3, 4, 5, 6, or 7 days. In some embodiments such a long-acting compstatin analog is a pegylated compstatin analog or a compstatin analog comprising a recombinant polypeptide. In some embodiments a long-acting compstatin analog comprises any of SEQ ID NOs: 3-41.

Exemplary long-acting compstatin analogs are described in any of the following: PCT/US2012/037648 (published as WO/2012/155107); U.S. Ser. No. 14/116,591 (published as US Pat. App. Pub. No. 20140323407); PCT/US2013/070424 (published as WO/2014/078734); PCT/US2013/070417 (published as WO/2014/078731); U.S. Ser. No. 14/443,143 (published as US Pat. Pub. No. 20160215020).

In some embodiments, a long-acting compstatin analog has an average plasma half-life of at least 1 day, e.g., 1-3 days, 3-7 days, 7-14 days, or 14-28 days, when administered IV at a dose of 10 mg/kg to humans or to non-human primates. In some embodiments, average plasma half-life of a long-acting compstatin analog following administration IV at a dose of 10 mg/kg to humans or to non-human primates is increased by at least a factor of 2, e.g., by a factor of 2-5, 5-10, 10-50, or 50-100-fold as compared with that of a corresponding compstatin analog having the same amino acid sequence (and, if applicable, one or more blocking moiet(ies)) but not comprising the CRM. In some embodiments, a plasma half-life is a terminal half-life after administration of a single IV dose. In some embodiments, a plasma half-life is a terminal half-life after steady state has been reached following administration of multiple IV doses.

The structure of compstatin is known in the art, and NMR structures for a number of compstatin analogs having higher activity than compstatin are also known (Malik, supra). Structural information may be used to design compstatin mimetics. In some embodiments, a compstatin mimetic is any compound that competes with compstatin or any compstatin analog (e.g., a compstatin analog whose sequence is set forth in Table 1) for binding to C3 or a fragment thereof (such as a 40 kD fragment of the β chain to which compstatin binds). In some embodiments, the compstatin mimetic has an activity equal to or greater than that of compstatin. In some embodiments, the compstatin mimetic is more stable, orally available, or has a better bioavailability than compstatin. The compstatin mimetic may be a peptide, nucleic acid, or small molecule. In certain embodiments the compstatin mimetic is a compound that binds to the binding site of compstatin as determined in a compstatin-C3 structure, e.g., a crystal structure or a 3-D structure derived from NMR experiments. In certain embodiments the compstatin mimetic is a compound that could substitute for compstatin in a compstatin-C3 structure and would form substantially the same intermolecular contacts with C3 as compstatin. In certain embodiments the compstatin mimetic is a compound that binds to the binding site of a peptide having a sequence set forth in Table 1, e.g., SEQ ID NO: 14, 21, 28, 29, 32, 33, 34, or 36 or other compstatin analog sequence or in certain embodiments SEQ ID NO: 30 or 31, in a peptide-C3 structure. In certain embodiments the compstatin mimetic is a compound that could substitute for a peptide having a sequence set forth in Table 1, e.g., SEQ ID NO: 14, 21, 28, 29, 32, 33, 34, or 36 or other compstatin analog sequence or in certain embodiments SEQ ID NO: 30 or 31, in a peptide-C3 structure and would form substantially the same intermolecular contacts with C3 as the peptide. In certain embodiments the compstatin mimetic has a non-peptide backbone but has side chains arranged in a sequence designed based on the sequence of compstatin.

One of skill in the art will appreciate that once a particular desired conformation of a short peptide has been ascertained, methods for designing a peptide or peptidomimetic to fit that conformation are well known. See, e.g., G. R. Marshall (1993), Tetrahedron, 49: 3547-3558; Hruby and Nikiforovich (1991), in Molecular Conformation and Biological Interactions, P. Balaram & S. Ramasehan, eds., Indian Acad. of Sci., Bangalore, PP. 429-455), Eguchi M, Kahn M., Mini Rev Med Chem., 2(5):447-62, 2002. Of particular relevance to the present invention, the design of peptide analogs may be further refined by considering the contribution of various side chains of amino acid residues, e.g., for the effect of functional groups or for steric considerations as described in the art for compstatin and analogs thereof, among others.

It will be appreciated by those of skill in the art that a peptide mimic may serve equally well as a peptide for the purpose of providing the specific backbone conformation and side chain functionalities required for binding to C3 and inhibiting complement activation. Accordingly, it is contemplated as being within the scope of the present invention to produce and utilize C3-binding, complement-inhibiting compounds through the use of either naturally-occurring amino acids, amino acid derivatives, analogs or non-amino acid molecules capable of being joined to form the appropriate backbone conformation. Anon-peptide analog, or an analog comprising peptide and non-peptide components, is sometimes referred to herein as a “peptidomimetic” or “isosteric mimetic,” to designate substitutions or derivations of a peptide that possesses much the same backbone conformational features and/or other functionalities, so as to be sufficiently similar to the exemplified peptides to inhibit complement activation. More generally, a compstatin mimetic is any compound that would position pharmacophores similarly to their positioning in compstatin, even if the backbone differs.

The use of peptidomimetics for the development of high-affinity peptide analogs is well known in the art. Assuming rotational constraints similar to those of amino acid residues within a peptide, analogs comprising non-amino acid moieties may be analyzed, and their conformational motifs verified, by means of the Ramachandran plot (Hruby & Nikiforovich 1991), among other known techniques.

One of skill in the art will readily be able to establish suitable screening assays to identify additional compstatin mimetics and to select those having desired inhibitory activities. For example, compstatin or an analog thereof could be labeled (e.g., with a radioactive or fluorescent label) and contacted with C3 in the presence of different concentrations of a test compound. The ability of the test compound to diminish binding of the compstatin analog to C3 is evaluated. A test compound that significantly diminishes binding of the compstatin analog to C3 is a candidate compstatin mimetic. For example, a test compound that diminishes steady-state concentration of a compstatin analog-C3 complex, or that diminishes the rate of formation of a compstatin analog-C3 complex by at least 25%, or by at least 50%, is a candidate compstatin mimetic. One of skill in the art will recognize that a number of variations of this screening assay may be employed. Compounds to be screened include natural products, libraries of aptamers, phage display libraries, compound libraries synthesized using combinatorial chemistry, etc. The invention encompasses synthesizing a combinatorial library of compounds based upon the core sequence described above and screening the library to identify compstatin mimetics. Any of these methods could also be used to identify new compstatin analogs having higher inhibitory activity than compstatin analogs tested thus far.

Other Compounds that Inhibit C3 Activation or Activity

Other compounds, e.g., polypeptides, small molecules, antibodies (e.g., monoclonal antibodies), aptamers, etc., that bind to C3 or C3a receptors (C3aR) are of use in certain embodiments of the invention. In certain embodiments the complement inhibitor comprises an Efb protein from Staphylococcus aureus or a variant or derivative or mimetic thereof that can bind to C3 and inhibit its activation and/or bind to and inhibit C3b. Exemplary agents are described in PCT Application Pub. WO/2004/094600. In certain embodiments the complement inhibitor comprises a Staphylococcus complement inhibitor (SCIN) protein from Staphylococcus aureus or a variant or derivative or mimetic of such protein that can bind to C3 convertase and inhibit its activation and/or bind to and inhibit C3b. Aptamers that bind to and inhibit C3 may be identified using methods such as SELEX. U.S. Pat. Pub. No. 20030191084 discloses aptamers that bind to C1q, C3 and C5. Exemplary antibodies that bind to C3 are disclosed in U.S. Pat. Pub. No. 20100111946 and/or 20120288493.

In some embodiments, a protease that degrades C3 may be used as a complement inhibitor. For example, U.S. Pat. No. 6,676,943 discloses human complement C3-degrading protein from Streptococcus pneumoniae. Such proteins, or variants thereof, may be used in certain embodiments of the invention. U.S. Pat. Pub. No. 20140242062 discloses variants of MT-SP1 protease. Such a protease may be used to degrade C3 in certain embodiments. In some embodiments the protease known as CB-2782 may be used.

U.S. Pat. No. 5,942,405, PCT/B2006/002557 (WO/2007/034277—ARYL SUBSTITUTED IMIDAZO [4,5-C] PYRIDINE COMPOUNDS AS C3A RECEPTOR ANTAGONISTS); PCT/IB2006/002568 (WO/2007/034282 —DIARYL-IMIDAZOLE COMPOUNDS CONDENSED WITH A HETEROCYCLE AS C3A RECEPTOR ANTAGONISTS) PCT/IB2006/002561 (WO2007034278—FUSED IMIDAZOLE DERIVATIVES AS C3A RECEPTOR ANTAGONISTS) PCT/US2007/026237 (WO2008079371) MODULATORS OF C3A RECEPTOR AND METHODS OF USE THEREOF disclose exemplary C3aR antagonists. In some embodiments, an RNAi agent that inhibits expression of C3 or C3aR may be used.

Compounds that Inhibit Factor B Activation or Activity

In certain embodiments a complement inhibitor inhibits activation or activity of factor B. For example, the complement inhibitor may bind to factor B and, e.g., inhibit activation of factor B. Exemplary agents that inhibit activation or activity of factor B include, e.g., antibodies (e.g., monoclonal antibodies), antibody fragments, peptides, small molecules, and aptamers. Exemplary antibodies that inhibit factor B are described in U.S. Pat. Pub. Nos. 20050260198 and 20130216529. In certain embodiments an antibody or antigen-binding fragment selectively binds to factor B within the third short consensus repeat (SCR) domain. In certain embodiments the antibody prevents formation of a C3bBb complex. In certain embodiments the antibody or antigen-binding fragment prevents or inhibits cleavage of factor B by factor D. In some embodiments, an antibody binds to the Bb portion of factor B. PCT/US2008/074489 (WO/2009/029669) discloses exemplary antibodies, e.g., the antibody produced by the hybridoma clone deposited under ATCC Accession Number PTA-8543. In some embodiments, a humanized version of said antibody is used, which may be an antibody fragment. In certain embodiments a complement inhibitor, e.g., antibody, small molecule, aptamer, polypeptide, or peptide, binds to substantially the same binding site on factor B as an antibody described in U.S. Pat. Pub. No. 20050260198 or WO/2009/029669. In some embodiments, the complement inhibitor comprises the monoclonal antibody fragment known as TA106 (formerly under development by Taligen Therapeutics), or antibody, small molecule, aptamer, polypeptide, or peptide, binds to substantially the same binding site on factor B as TA106 is used. In some embodiments, a peptide that binds to and inhibits factor B is identified using, for example, a method such as phage display. In some embodiments, a complement inhibitor comprises an aptamer that binds to and inhibits factor B. In some embodiments, an RNAi agent that inhibits expression of factor B may be used.

Compounds that Inhibit Factor D Activity

In certain embodiments the complement inhibitor inhibits factor D. For example, the complement inhibitor may bind to factor D. Exemplary agents include antibodies (e.g., monoclonal antibodies), antibody fragments, peptides, small molecules, and aptamers. Exemplary antibodies that inhibit factor D are described in U.S. Pat. No. 7,112,327. In certain embodiments the complement inhibitor is an antibody, small molecule, aptamer, or polypeptide that binds to substantially the same binding site on factor D as an antibody described in U.S. Pat. No. 7,112,327. FCFD4514S (formerly under development by Tanox as TNX-234, now under development for by Genentech as lampalizumab), is a humanized monoclonal antibody fragment that binds Factor D. In certain embodiments the complement inhibitor comprises FCFD4514S or an antibody, small molecule, aptamer, or polypeptide that binds to substantially the same binding site on factor D as FCFD4514S. Exemplary polypeptides that inhibit alternative pathway activation and are believed to inhibit factor D are disclosed in U.S. Pub. No. 20040038869. Exemplary small molecules that inhibit factor D are disclosed in U.S. Pat. Pub. Nos. 20150239837, 20150239838, 20150239919. In some embodiments eptides that bind to and inhibit factor D, which may be identified using methods such as phage display, or aptamers that bind to and inhibit factor D, which may be identified using methods such as SELEX, may be used. In some embodiments, an RNAi agent that inhibits expression of factor D may be used.

Mammalian Complement Regulatory Proteins and Complement Receptors

In some embodiments the complement inhibitor comprises at least a portion of a mammalian, e.g., human, complement regulatory protein or complement receptor. Examples of complement regulatory proteins include, e.g., CFH, CFH related proteins (such as CFHR1), CFI, CR1, DAF, MCP, CD59, and C4 bp. In some embodiments the complement regulatory polypeptide is one that is normally membrane-bound in its naturally occurring state. In some embodiments of the invention a fragment of such polypeptide that lacks some or all of a transmembrane and/or intracellular domain is used. Soluble forms of complement receptor 1 (sCR1), or soluble portions of other complement receptors, for example, are of use in certain embodiments. For example the compounds known as TP10 or TP20 (Avant Therapeutics) can be used. In some embodiments a soluble complement control protein, e.g., CFH or a CFH related protein, is used. In some embodiments the complement inhibitor is a C3b/C4b Complement Receptor-like molecule such as those described in U.S. Pat. Pub. No. 20020192758. Variants and fragments of mammalian complement regulatory proteins or receptors that retain complement inhibiting activity can be used in certain embodiments. In some embodiments an engineered polypeptide derived in part from CHF may be used, such as those described in WO2013142362, e.g., comprising a first domain comprising CCP modules 1-4, which mediates C3b binding and exerts regulatory activities, and a second domain comprising CCP modules 19-20, which enhances binding to C3b and allows recognition of self-surfaces. The domains may be linked using a linker, such as a glycine linker.

In certain embodiments of the invention the complement inhibitor comprises a chimeric polypeptide comprising a first polypeptide that inhibits complement activation, linked, e.g., covalently linked, to a second polypeptide that inhibits complement activation and/or that binds to a complement component or complement activation product. In some embodiments, at least one of the polypeptides comprises at least a portion of a mammalian complement regulatory protein. The chimeric polypeptide may contain one or more additional domains located, e.g., between the first and second polypeptides or at a terminus. For example, the first and second polypeptides can be separated by a spacer polypeptide.

In some embodiments, the first and second polypeptides each comprise at least a portion of a mammalian complement regulatory protein. In some embodiments complement inhibitor comprises at least a portion of DAF and at least a portion of MCP. Exemplary chimeric polypeptides are disclosed, e.g., in U.S. Pat. No. 5,679,546, e.g., CAB-2 (also known as MLN-2222). In some embodiments the polypeptide comprises at least 4 SCR domains of at least one mammalian complement regulatory protein or complement receptor. In some embodiments the polypeptide comprises at least 4 SCR domains of each of first and second distinct mammalian complement regulatory proteins.

In some embodiments, a chimeric polypeptide comprises at least a portion of complement receptor 1 (CR1), complement receptor 2 (CR2), complement receptor 3 (CR3), complement receptor 4 (CR4) or a variant or fragment of CR1, CR2, CR3, or CR4 that binds to one or more complement components or complement activation products such as C3b, iC3b, C3d, and/or C3dg. In some embodiments, the polypeptide comprises at least 4 SCRs, e.g., at least 4 SCRs of CR1 or CR2. For example, the polypeptide can comprise the 4 N-terminal SCRs of CR2 (e.g., residues 1-250 of the mature protein). In some embodiments the chimeric polypeptide comprises at least 4 SCR domains of a mammalian complement regulatory protein and at least 4 SCR domains of a mammalian complement receptor.

Compounds that Inhibit Properdin

In some embodiments of the invention antiproperdin antibodies (e.g., monoclonal antibodies), antibody fragments, or other anti-properdin agents are used. See, e.g., U.S. Pat. Pub. Nos. 20030198636, 20110008340 or 20130295102 for examples.

Compounds that Inhibit Lectin Pathway Components

In some embodiments the compounds inhibit one or more components of the lectin pathway. Such compounds include, e.g., antibodies, (e.g., monoclonal antibodies), antibody fragments, polypeptides, small molecules, and aptamers that bind to one or more components of the lectin pathway, e.g., MASP-1, MASP-2, and/or MASP-3; peptides that compete with MASP-1 for binding to MASP-3; compounds that inhibit expression of one or more lectin pathway components (e.g., siRNA or antisense agents). See, e.g., WO/2007/117996, WO/2013/192240, US Patent Application Pub. Nos. 20070172483 and 20130344073. Peptide inhibitors of MASP-1 and MASP-2, termed SGMI-1 and SGMI-2, respectively, are described in Heja et al., J Biol Chem 287:20290 (2012) and Heja et al., PNAS 109: 10498 (2012). SGMI-1 and SGMI-2 are each 36 amino acid peptides that block the lectin pathway of complement activation without affecting the classical or alternative pathways (Heja et al., supra). The amino acid sequences of the SGMI-1 and SGMI-2 inhibitors are as follows: SGMI-1-full-length: LEVTCEPGTTFKDKCNTCRCGSDGKSAFCTRKLCYQ (SEQ ID NO:XX) SGMI-2-full-length: LEVTCEPGTTFKDKCNTCRCGSDGKSAVCTKLWCNQ (SEQ ID NO:XX). In some embodiments, a peptide such as SGMI-1 or SGMI-2 is attached to a clearance reducing moiety, e.g., a polypeptide such as an Ig Fc domain, human serum albumin, an albumin binding peptide, or a PEG or POZ.

Compounds that Inhibit C5 Activation or Activity

In certain embodiments the complement inhibitor inhibits activation of C5. For example, the complement inhibitor may bind to C5 and inhibit its cleavage. In some embodiments, the complement inhibitor inhibits physical interaction of C5 with C5 convertase by, e.g., binding to C5 or C5 convertase or to C5 at a site that would ordinarily participate in such physical interaction. Exemplary agents that inhibit C5 activation include antibodies (e.g., monoclonal antibodies), antibody fragments, polypeptides, small molecules, and aptamers. Exemplary compounds, e.g., antibodies, that bind to C5 are described, for example, in U.S. Pat. No. 6,534,058; PCT/US95/05688 (WO 1995/029697), PCT/EP2010/007197 (WO2011063980); U.S. Pat. Pub. Nos. 20050090448; 20060115476, 20100034809, 20120237515, 20160251433, and 20170073399. Exemplary polypeptides that bind to C5 are disclosed in U.S. Pat. Pub. No. 20170137468, W2016123371, and WO2017105939. U.S. Pat. Pub. No. 20060105980 discloses aptamers that bind to and inhibit C5. In some embodiments, a humanized anti-C5 monoclonal antibody, e.g., eculizumab (also known as h5G1.1-mAb; Soliris®) (Alexion), or a fragment or derivative thereof that binds to C5, is used. In some embodiments, an antibody comprising at least some of the same complementarity determining regions (CDR1, CDR2 and/or CDR3), e.g., all of CDR1, CDR2, and CDR3, as those of eculizumab's heavy chain and/or light chain is used. In some embodiments, the antibody comprises at least some of the same framework regions as eculizumab. In some embodiments, an antibody that binds to substantially the same binding site on C5 as eculizumab is used. In some embodiments, an antibody that is a variant of eculizumab engineered to have a longer half-life than eculizumab is used. Examples of such variants are described in U.S. Pat. Pub. No. 20160251433. In some embodiments the eculizumab variant referred to as BNJ441 (also known as ALXN-1210) may be used. In some embodiments, an antibody referred to as is antibody 8109 (see, e.g., US Pat. Pub. No. 20100034809) or comprising the same CDRs as antibody 8109 is used. In some embodiments, pexelizumab (also known as h5G1.1-scFv), a humanized, recombinant, single-chain antibody derived from h5G1.1-mAb, is used.

In certain embodiments the complement inhibitor comprises an antibody that binds to C5a. Exemplary compounds, e.g., antibodies, that bind to C5a are described, for example, in U.S. Pat. Pub. No. 20130224187

In certain embodiments the complement inhibitor comprises a Staphylococcus SSL7 protein from Staphylococcus aureus or a variant or derivative or mimetic of such protein that can bind to C5 and inhibit its cleavage.

As noted above, bispecific or multispecific antibodies can be used. For example, PCT/US2010/039448 (WO/2010/151526) discloses bispecific antibodies described as binding to two or more different proteins, wherein at least two of the proteins are selected from C5a, C5b, a cellular receptor for C5a (e.g., C5aR1 or C5L2), the C5b-9 complex, and a component or intermediate of terminal complement such as C5b-6, C5b-7, or C5b-8. In some embodiments an RNAi agent that inhibits expression of C5 or C5aR may be used.

In some embodiments, a complement inhibitor known as OmCI, or a variant, derivative, or mimetic thereof, is used. OmCI binds to C5 and inhibits its activation most likely by inhibiting interaction with convertase. OmCI is naturally produced by the tick Ornithodoros moubata. See, e.g., PCT/GB2004/002341 (WO/2004/106369) and PCT/GB2010/000213 (WO/2010/100396), for description of OmCI and certain variants thereof. It has been shown that OmCI binds to eicosanoids, in particular leukotriene (LKs), e.g., LTB4. In some embodiments, an OmCI polypeptide (or a variant, derivative, or fragment thereof) that retains the capacity to binds to a LK, e.g., LTB4, is used. In some embodiments, an OmCI polypeptide (or a variant, derivative, or fragment thereof) that has reduced capacity or substantially lacks capacity to bind to a LK, e.g., LTB4, is used.

In some embodiments the agent is an antagonist of a C5a receptor (C5aR). In some embodiments, the C5aR antagonist comprises a peptide. Exemplary C5a receptor antagonists include a variety of small cyclic or acyclic peptides such as those described in March, D R, et al., Mol. Pharmacol., 65(4), 2004, and in Woodruff, T M, et al., J Pharmacol Exp Ther., 314(2):811-7, 2005, U.S. Pat. No. 6,821,950; U.S. Ser. No. 11/375,587; and/or PCT/US06/08960 (WO2006/099330), or a mimetic thereof. In certain embodiments the complement inhibitor binds to C5aR and inhibits binding of C5a thereto. In certain embodiments a cyclic peptide comprising the sequence [OPdChaWR] (SEQ ID NO: 59) is used. In certain embodiments a cyclic peptide comprising the sequence [KPdChaWR] (SEQ ID NO: 60) is used. In certain embodiments a peptide comprising the sequence (Xaa)ₙ[OPdChaWR] (SEQ ID NO: 61) is used, wherein Xaa is an amino acid residue and n is between 1 and 5. In certain embodiments a peptide comprising the sequence (Xaa)ₙ[KPdChaWR] (SEQ ID NO: 62) is used, wherein Xaa is an amino acid residue and n is between 1 and 5. In certain embodiments n is 1. In certain embodiments n is 1 and Xaa is a standard or nonstandard aromatic amino acid. For example, the peptides F-[OPdChaWR](SEQ ID NO: 63), F-[KPdChaWR] (SEQ ID NO: 64); Cin-[OPdChaWR] (SEQ ID NO: 65), and HCin-[OPdChaWR] (SEQ ID NO: 66) are of use in certain embodiments. Optionally the free terminus comprises a blocking moiety, e.g., the terminal amino acid is acetylated. For example, in some embodiments the C5aR antagonist is AcF-[OPdChaWR] (SEQ ID NO: 67) (also known as PMX-53). (Abbreviations: O: omithine; Cha: cyclohexylalanine; Cin: cinnamoyl; Hcin: hydrocinnamoyl; square brackets denote internal peptide bond). In some embodiments, a C5aR antagonist comprises a compound, e.g., a peptide, disclosed in U.S. Pat. Pub. No. 20060183883 (U.S. Ser. No. 10/564,788), e.g., a compound as represented therein by formula I, formula II, formula IV, formula V, or formula VI. An exemplary C5aR antagonist is the peptide known as JPE-1375 (Jerini A G, Germany).

In some embodiments, a C5aR antagonist is a small molecule. Various small molecule C5aR antagonists are disclosed in the following references: PCT/US2005/015897 (WO/2005/110416; 4,5-DISUBSTITUTED-2-ARYL PYRIMIDINES); PCT/EP2006/005141 (WO2006128670); PCT/US2008/072902 (WO/2009/023669; SUBSTITUTED 5,6,7,8-TETRAHYDROQUINOLINE DERIVATIVES); PCT/US2009/068941 (WO/2010/075257; C5AR ANTAGONISTS). An exemplary small molecule C5aR antagonist is CCX168 (ChemoCentryx, Mountain View, Calif.).

In certain embodiments the complement inhibitor is an agent, e.g., an antibody, small molecule, aptamer, or polypeptide, that binds to substantially the same binding site on C5 or C5aR as a compound described in any of the afore-mentioned references disclosing agents that bind to C5 or C5aR.

In some embodiments the complement inhibitor is not an antagonist of a C5a receptor.

Multimodal Complement Inhibitors

In certain embodiments of the invention the complement inhibitor binds to more than one complement protein and/or inhibits more than one step in a complement activation pathway. Such complement inhibitors are referred to herein as “multimodal”. In certain embodiments of the invention the complement inhibitor comprises a virus complement control protein (VCCP). The invention contemplates use of any of the agents described in U.S. Ser. No. 11/247,886 and PCT/US2005/36547. Poxviruses and herpesviruses are families of large, complex viruses with a linear double-stranded DNA genome. Certain of these viruses encode immunomodulatory proteins that are believed to play a role in pathogenesis by subverting one or more aspects of the normal immune response and/or fostering development of a more favorable environment in the host organism (Kotwal, G J, Immunology Today, 21(5), 242-248, 2000). Among these are VCCPs. Poxvirus complement control proteins are members of the complement control protein (CCP) superfamily and typically contain 4 SCR modules. In certain embodiments the VCCP is a poxvirus complement control protein (PVCCP). The PVCCP can comprise a sequence encoded by, e.g., vaccinia virus, variola major virus, variola minor virus, cowpox virus, monkeypox virus, ectromelia virus, rabbitpox virus, myxoma virus, Yaba-like disease virus, or swinepox virus. In some embodiments the VCCP is a herpesvirus complement control protein (HVCCP). The HVCCP can comprise a sequence encoded by a Macaca fuscata rhadinovirus, cercopithecine herpesvirus 17, or human herpes virus 8. In some embodiments the HVCCP comprises a sequence encoded by herpes simplex virus saimiri ORF 4 or ORF 15 (Albrecht, J C. & Fleckenstein, B., J. Virol., 66, 3937-3940, 1992; Albrecht, J., et al., Virology, 190, 527-530, 1992).

The VCCP may inhibit the classical complement pathway, the alternate complement pathway, the lectin pathway, or any two or more of these. In certain embodiments of the invention the VCCP, e.g., a PVCCP, binds to C3b, C4b, or both. In certain embodiments of the invention the PVCCP comprises one or more putative heparin binding sites (K/R-X-K/R) and/or possesses an overall positive charge. In some embodiments the PVCCP comprises at least 3 SCR modules (e.g., modules 1-3), e.g., 4 SCR modules. The PVCCP protein can be a precursor of a mature PVCCP (i.e., can include a signal sequence that is normally cleaved off when the protein is expressed in virus-infected cells) or can be a mature form (i.e., lacking the signal sequence).

Vaccinia complement control protein (VCP) is a virus-encoded protein secreted from vaccinia infected cells. VCP is 244 amino acids in length, contains 4 SCRs, and is naturally produced by intracellular cleavage of a 263 amino acid precursor. VCP runs as an ˜35 kD protein in a 12% SDS/polyacrylamide gel under reducing conditions and has a predicted molecular mass of about 28.6 kD. VCP is described in U.S. Pat. Nos. 5,157,110 and 6,140,472, and in Kotwal, G K, et al., Nature, 355, 176-178, 1988. FIGS. 3A and 3B of U.S. Ser. No. 11/247,886 and PCT/US2005/36547 (WO2006042252) show the sequence of the precursor and mature VCP proteins, respectively. VCP has been shown to inhibit the classical pathway of complement activation via its ability to bind to C3 and C4 and act as a cofactor for factor I mediated cleavage of these components as well as promoting decay of existing convertase (Kotwal, G K, et al., Science, 250, 827-830, 1990; McKenzie et al., J. Infect. Dis., 1566, 1245-1250, 1992). It has also been shown to inhibit the alternative pathway by causing cleavage of C3b into iC3b and thereby preventing the formation of the alternative pathway C3 convertase (Sahu, A, et al., J. Immunol., 160, 5596-5604, 1998). VCP thus blocks complement activation at multiple steps and reduces levels of the proinflammatory chemotactic factors C3a, C4a, and C5a.

VCP also possesses the ability to strongly bind heparin in addition to heparan sulfate proteoglycans. VCP contains two putative heparin binding sites located in modules 1 and 4 (Jha, P and Kotwal, G J, and references therein). VCP is able to bind to the surface of endothelial cells, possibly via interaction with heparin and/or heparan sulfate at the cell surface, resulting in decreased antibody binding (Smith, S A, et al., J Virol., 74(12), 5659-5666, 2000). VCP can be taken up by mast cells and possibly persist in tissue for lengthy periods of time, thereby potentially prolonging its activity (Kotwal, G J, et al., In G P. Talwat, et al. (eds), 10^{th} International Congress of Immunology., Monduzzi Editore, Bologna, Italy, 1998). In addition, VCP can reduce chemotactic migration of leukocytes by blocking chemokine binding (Reynolds, D, et al., in S. Jameel and L. Villareal (ed., Advances in animal virology. Oxford and IBN Publishing, New Delhi, India, 1999). VCP and other PVCCPs have a relatively small size relative to mammalian CCPs, which is advantageous for delivery in the present invention.

Variola virus major and minor encode proteins that are highly homologous to VCP and are referred to as smallpox inhibitor of complement enzymes (SPICE) (Rosengard, A M, et al., Proc. Natl. Acad. Sci., 99(13), 8803-8813. U.S. Pat. No. 6,551,595). SPICE from various variola strains sequenced to date differs from VCP by about 5% (e.g., about 11 amino acid differences). Similarly to VCP, SPICE binds to C3b and C4b and causes their degradation, acting as a cofactor for factor I. However, SPICE degrades C3b approximately 100 times as fast as VCP and degrades C4b approximately 6 times as fast as VCP. The amino acid sequence of SPICE is presented in FIG. 6 (SEQ ID NO: 12) of U.S. Ser. No. 11/247,886 and PCT/US2005/36547 (WO2006042252) and can be described as follows. Referring to FIG. 6 of U.S. Ser. No. 11/247,886 and PCT/US2005/36547 (WO2006042252), a signal sequence extends from amino acid 1 to about amino acid 19. Four SCRs extend from about amino acid 20 to amino acid 263. Each SCR is characterized by four cysteine residues. The four cysteine residues form two disulfide bonds in the expressed protein. The boundaries of each SCR are best defined by the first and fourth cysteine residues in the sequence that forms the disulfide bonds of the SCR. An invariant tryptophan residue is present between cysteine 3 and cysteine 4 of each SCR. SCR1 extends from amino acid 20 or 21 to amino acid 81. Both residues are cysteines that may be involved in disulfide bonding. SCR2 extends from amino acid 86 to amino acid 143. SCR3 extends from amino acid 148 to amino acid 201. SCR4 extends from amino acid 206 to amino acid 261. The SCRs include the complement binding locations of SPICE. SPICE or any of the portions thereof that inhibit complement activation, e.g., SPICE and SPICE-related polypeptides containing four SCRs, such as those described in U.S. Pat. No. 6,551,595, are of use in the present invention.

Complement control proteins from cowpox virus (referred to as inflammation modulatory protein, IMP) and monkeypox virus (referred to herein as monkeypox virus complement control protein, MCP) have also been identified and sequenced (Miller, C G, et al., Virology, 229, 126-133, 1997 and Uvarova, E A and Shchelkunov, S N, Virus Res., 81(1-2), 39-45, 2001). MCP differs from the other PVCCPs described herein in that it contains a truncation of the C-terminal portion of the fourth SCR.

It will be appreciated that the exact sequence of complement control proteins identified in different virus isolates may differ slightly. Such proteins fall within the scope of the present invention. Complement control proteins from any such isolate may be used, provided that the protein has not undergone a mutation that substantially abolishes its activity. Thus the sequence of a VCCP such as SPICE or VCP may differ from the exact sequences presented herein or under the accession numbers listed in Table 2. It will also be appreciated that a number of amino acid alterations, e.g., additions, deletions, or substitutions such as conservative amino acid substitutions, may be made in a typical polypeptide such as a VCCP without significantly affecting its activity, such that the resulting protein is considered equivalent to the original polypeptide. The viral polypeptides identified by accession number in Table 2 below are of use in various embodiments of the invention.

TABLE 2
Representative Viral Complement Control Proteins

| Virus | Protein | Accession | Virus Type |
|---|---|---|---|
| Variola | D12L | NP_042056 | Orthopoxvirus |
| | D15L (SPICE) | AAA69423 | Orthopoxvirus |
| Vaccinia | VCP | AAO89304 | Orthopoxvirus |
| Cowpox | CPXV034 | AAM13481 | Orthopoxvirus |
| | C17L | CAA64102 | Orthopoxvirus |
| Monkeypox | D14L | AAV84857 | Orthopoxvirus |
| Ectromelia | Complement control | CAE00484 | Orthopoxvirus |
| virus | protein | | |
| Rabbitpox | RPXV017 | AAS49730 | Orthopoxvirus |
| Macaca fuscata | JM4 | AAS99981 | Rhadinavirus |
| rhadinovirus | | | (Herpesvirus) |
| Cercopithecine | Complement binding | NP_570746 | Herpesvirus |
| herpesvirus 17 | protein (ORF4) | | |
| Human herpes | Complement binding | AAB62602 | Herpesvirus |
| virus 8 | protein (ORF4) | | |

In addition to the VCCPs described above, a number of other viral proteins exist that interfere with one or more steps in a complement pathway. These proteins are also of use in certain embodiments of the present invention. Certain of these proteins do not necessarily display clear homology to cellular complement regulators known to date. For example, HSV-1, HSV-2, VZV, PRV, BHV-1, EHV-1, and EHV-4 all encode versions of a conserved glycoprotein known as gC (Schreurs, et al., J Virol., 62, 2251-2257, 1988; Mettenleiter, et al, J Virol., 64, 278-286; 1990; Herold, et al., J Virol., 65, 1090-1098; 1991). With the exception of VZV, the gC protein encoded by these viruses binds to C3b (Friedman, et al., Nature, 309, 633-634, 1984; Huemer, et al., Virus Res., 23, 271-280, 1993) gC1 (from HSV-1) accelerates decay of the classical pathway C3 convertase and inhibits binding of properdin and C5 to C3. Purified EBV virions possess an activity that accelerates decay of the alternative pathway C3 convertase and serves as a cofactor for the complement regulatory protein factor 1 (Mold et al., J Exp Med, 168, 949-969, 1988). The foregoing proteins are referred to collectively as virus complement interfering proteins (VCIPs). By any of a variety of means, such as interfering with one or more steps of complement activation, accelerating decay of a complement component, and/or enhancing activity of a complement regulatory protein, these VCIPs are said to inhibit complement. Any of these proteins, or derivatives thereof, e.g., fragments or variants thereof, can be used as a therapeutic agent in the invention. As in the case of VCCPs, will be appreciated that the exact sequence of VCIPs identified in different virus isolates may differ slightly. Such proteins fall within the scope of the present invention.

In certain embodiments of the invention a fragment or variant of a VCCP or VCIP is locally administered to a subject. Preferred fragments and variants of a PVCCP possess at least one of the following activities: (i) ability to bind to C3, C3b, or both; (ii) ability to act as a cofactor for factor I cleavage of C3; (iii) ability to bind to C4, C4b, or both; (iv) ability to act as a cofactor for factor I cleavage of C4; (v) ability to accelerate decay of existing C3 convertase of the classical pathway, alternate pathway, or both; (vi) ability to bind heparin; (vii) ability to bind to heparan sulfate proteoglycans; (viii) ability to reduce chemotactic migration of leukocytes; (ix) ability to block chemokine (e.g, MIP-1α) binding, e.g., to the surface of a cell (e.g., a leukocyte or endothelial cell surface); (x) ability to inhibit antibody binding to class I MHC molecules; (xi) ability to inhibit the classical complement pathway; (xii) ability to inhibit the alternative complement pathway; and (xiii) ability to inhibit complement-mediated cell lysis. Preferred PVCCP fragments and variants display complement binding activity, by which is meant ability to detectably bind to one or more complement components, preferably (in the case of VCCPs) selected from the group consisting of: C3, C3b, C4, and C4b. Preferred fragments or variants of HVCCPs may also display ability to detectably bind to one or more complement components. Preferably the binding of the VCCP to the complement component is specific. It will be understood that a VCCP may be able to bind to only a single complement component or may be able to bind to more than one different complement component.

In certain embodiments of the invention the PVCCP fragment or variant comprises at least 3 SCR modules (e.g., modules 1-3), preferably 4 SCR modules. Preferably each of the SCR modules displays significant sequence identity to an SCR module found in a naturally occurring PVCCP, e.g., VCP or SPICE. Preferably the multiple SCR modules are arranged in an N to C manner so as to maximize overall identity to a naturally occurring PVCCP. If the sequence of a PVCCP fragment or variant contains an SCR domain that differs from the SCR consensus sequence at one or more positions, in certain embodiments of the invention the amino acid(s) at the one or more differing positions is identical to that found at a corresponding position in the most closely related SCR found in a naturally occurring PVCCP. In certain embodiments the PVCCP variant comprises at least one SCR module from a first PVCPP and at least one SCR module from a second PVCPP. In certain embodiments the PVCCP variant comprises at least one SCR module from a PVCCP and at least one SCR from a mammalian complement control protein (RCA protein). Any number of SCR modules, e.g., 1, 2, 3, 4, or more can come from any particular PVCCP or RCA protein in various embodiments of the invention. All such combinations and permutations are contemplated, even if not explicitly set forth herein.

Generally a fragment or variant of a naturally occurring VCCP or VCIP possesses sufficient structural similarity to its naturally occurring counterpart that it is recognized by a polyclonal antibody that recognizes the naturally occurring counterpart. In certain embodiments of the invention a fragment or variant of a VCCP possesses sufficient structural similarity to VCP or SPICE so that when its 3-dimensional structure (either actual or predicted structure) is superimposed on the structure of VCP or SPICE, the volume of overlap is at least 70%, preferably at least 80%, more preferably at least 90% of the total volume of the VCP structure. A partial or complete 3-dimensional structure of the fragment or variant may be determined by crystallizing the protein as described for VCP (Murthy, 2001). Alternately, an NMR solution structure can be generated, as performed for various VCP fragments (Wiles, A P, et al., J. Mol. Biol. 272, 253-265, 1997). A modeling program such as MODELER (Sali, A. and Blundell, T L, J Mol. Biol., 234, 779-815, 1993), or any other modeling program, can be used to generate a predicted structure. The model can be based on the VCP structure and/or any known SCR structure. The PROSPECT-PSPP suite of programs can be used (Guo, J T, et al., Nucleic Acids Res. 32 (Web Server issue):W522-5, Jul. 1, 2004). Similar methods may be used to generate a structure for SPICE.

Fragments or variants of a VCCP or VCIP may be generated by any available means, a large number of which are known in the art. For example, VCCPs, VCIPs, and fragments or variants thereof can be produced using recombinant DNA technology as described below. A VCCP or VCIP fragment may be chemically synthesized, produced using PCR amplification from a cloned VCCP or VCIP sequence, generated by a restriction digest, etc. Sequences for a VCCP variant may be generated by random mutagenesis of a VCCP sequence (e.g., using X-rays, chemical agents, or PCR-based mutagenesis), site-directed mutagenesis (e.g., using PCR or oligonucleotide-directed mutagenesis, etc. Selected amino acids can be changed or added.

While not wishing to be bound by any theory, it is likely that amino acid differences between naturally occurring PVCCPs occur at positions that are relevant in conferring differences in particular properties such as ability to bind heparin, activity level, etc. For example, VCP and SPICE differ at only 11 amino acids, but SPICE has a much higher activity as a cofactor for cleavage of C3b (e.g., cleavage occurs at a much faster rate with SPICE than with VCP). The amino acid differences are likely to be responsible for the differential activities of the two proteins. The amino acids at these positions are attractive candidates for alteration to identify variants that have yet greater activity.

Additional Complement Inhibitors

In some embodiments a complement inhibitor is a naturally occurring mammalian complement regulatory protein or a fragment or derivative thereof. For example, the complement regulatory protein may be CR1, DAF, MCP, CFH, or CFI. In some embodiments of the invention the complement regulatory polypeptide is one that is normally membrane-bound in its naturally occurring state. In some embodiments of the invention a fragment of such polypeptide that lacks some or all of a transmembrane and/or intracellular domain is used. Soluble forms of complement receptor 1 (sCRI), for example, are of use in the invention. For example the compounds known as TP10 or TP20 (Avant Therapeutics) can be used. C1 inhibitor (C1-INH) is also of use. In some embodiments a soluble complement control protein, e.g., CFH, is used. In some embodiments of the invention the polypeptide is modified to increase its solubility.

In some embodiments, a complement inhibitor is a C1s inhibitor. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 68) or KTISKAKGQPREPQVYT (SEQ ID NO: 69) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

Bifunctional Complement Inhibitors

In some embodiments a complement inhibitor is a bifunctional complement inhibitor comprising a first moiety that inhibits a first complement component and a second moiety that inhibits a second complement component. In some embodiments the first moiety inhibits C3 and a second moiety that inhibits the C5a receptor. In some embodiments of the invention the first moiety inhibits C3 and a second moiety that inhibits the C3a receptor. In some embodiments of the invention the first moiety inhibits C3 and a second moiety that inhibits the C5a receptor.

Complement System Assays and Biomarkers

In some aspects, complement activation may be measured using, e.g., a suitable assay such as a functional assay based on hemolysis (e.g., lysis of sheep or chicken red blood cells); deposition or capture of complement activation products (e.g., C3a, C3b, iC3b, C5a, MAC), etc. Pathway-specific complement activation capacity may be assessed using, e.g., appropriate stimuli and assay conditions (e.g., presence or absence of calcium ions in the assay composition) to activate one or more than one of the pathways. For example, antibody (e.g., IgM or immune complex) can be used to activate the classical pathway; lipopolysaccharide (LPS) can be used to activate the alternative pathway, mannan can be used to activate the mannose-binding lectin portion of the lectin pathway, etc. In some embodiments, the total classical complement activity in a sample is measured using a CH50 test using antibody-sensitized sheep or chicken erythrocytes as the activator of the classical complement pathway and various dilutions of the test sample to determine the amount required to give 50% lysis. The percent hemolysis can be determined spectrophotometrically. The higher the dilution of the sample that can still achieve 50% lysis (i.e., the more diluted the sample), the greater complement activation capacity. In some embodiments, an ELISA-based assay is used. In some embodiments, complement activation is assessed based on iC3b levels, e.g., substantially as described in PCT/US2010/035871 (WO2010135717) (see Examples). In some embodiments, complement activation is assessed based on C3b levels, substantially as described in PCT/US2008/001483 (WO/2008/097525) Examples 1 and 2, respectively. In some embodiments, complement activation via the classical pathway is assessed using the MicroVue CH50 Eq EIA Kit (classical pathway), MicroVue Bb Plus EIA Kit (alternative pathway), MicroVue iC3b EIA Kit, or MicroVue C3a Plus EIA Kit (all from Quidel Corp.). In some embodiments, the amount of a complement activation product is normalized to the amount of intact C3 present in the sample prior to exposure to a complement activation stimulus. In some embodiments, complement activity, complement activation, or complement activation capacity, or the effect of a complement inhibitor, may be measured as described in any of the following US Patent App. Pub. Nos.: US20100166862, US20120141457, US 20120315266. In some embodiments, complement component C3 and the activation fragment C3d are measured in serum samples (e.g., as described in Smailhodzic D., et al., Ophthalmology, 119: 339-346.). The C3d/C3 ratio is calculated as a measure of C3 activation.

In some embodiments, a complement system biomarker level is measured in a sample obtained from a subject. In some embodiments a sample comprises a body fluid, e.g., blood, BAL fluid, sputum, nasal secretion, urine, etc. In some embodiments a sample comprises a tissue sample, which may be obtained from a cancer. In some embodiments a level is compared with a reference value. In some embodiments a reference value may be a normal value (e.g., a value within a normal range, e.g., an upper limit of a normal range). In some embodiments, if a measured value deviates significantly from a reference value or shows a trend towards increased deviation from a reference value in a manner indicative of increased complement activation, the subject may be considered to be an appropriate candidate for treatment with a complement inhibitor. A “normal range” may be a range that encompasses at least 95% of healthy individuals. In some embodiments a reference value or reference range may be a value or range associated with a disease in which complement activation plays a role, e.g., a value or range typically found in subjects suffering from such a disease in an untreated state. In such embodiments, a subject in whom the value is near or within the reference value or range may be considered to be an appropriate candidate for treatment with a complement inhibitor. In some embodiments a normal or disease-associated range may depend at least in part on demographic factors such as age, gender, etc., and can be adjusted accordingly. An appropriate reference value or range may be established empirically for different disorders and/or different biomarkers and/or, in some embodiments, for individual subjects.

In some embodiments, in vivo assessment of a complement system biomarker is envisioned. For example, in some embodiments a detectably labeled agent that binds to a product of complement activation administered to a subject. A suitable imaging method is used to visualize the agent in vivo. In some embodiments, for example, an image is obtained of the lungs, skin, or other location that may be affected by a complement-mediated disorder. In some embodiments in vivo detection allows assessment of the immunological microenvironment in a tumor, tissue, or organ of interest. In some embodiments a detectable label comprises a fluorescent, radioactive, ultrasound, or magnetically detectable moiety. In some embodiments an imaging method comprises magnetic resonance imaging, ultrasound imaging, optical imaging (e.g., fluorescence imaging or bioluminescence imaging), or nuclear imaging. In some embodiments a fluorescent moiety comprises a near-infrared or infrared fluorescent moiety (emitting in the near-infrared or infrared region of the spectrum). In some embodiments an imaging method comprises positron emission tomography (PET), and single photon emission computed tomography (SPECT). In some embodiments a detectable label is attached to an agent that binds directly to a target to be detected. In some embodiments a detectable label is associated with or incoporated into or comprises particles, which in some embodiments have at their surface an agent that binds directly to a target to be detected. PCT/US2013/055394 describes certain in vivo methods of detecting complement activation.

### V. Multifunctional iC3b/C3dg/C3d Inhibitors/Complement Inhibitors

In some aspects, described herein is a multifunctional agent comprising a first moiety comprising an iC3b/C3dg/C3d inhibitor and a second moiety comprising a complement inhibitor. All combinations of iC3b/C3dg/C3d inhibitor and complement inhibitor are specifically encompassed. In general, any suitable method for preparing conjugates or fusion proteins may be used to generate a multifunctional agent. In certain embodiments any of the methods of preparing conjugates described in Hermanson, supra and/or in PCT/US2012/054180 (published as WO/2013/036778); PCT/US2012/037648 (published as WO/2012/155107); U.S. Ser. No. 14/116,591; PCT/US2013/070424 (published as WO/2014/078734); PCT/US2013/070417 (published as WO/2014/078731) may be used. Two moieties may be directly conjugated or fused to each other or may be joined by a linking moiety. It will be understood that modifications may be made to either or both of the agents in order to attach them to each other or to a linking moiety, such as addition of a moiety comprising a reactive functional group, and the linking portion (e.g., comprising one or more covalent bonds) created by the linkage.

Two moieties may be directly linked to each other covalently or noncovalently or may be linked to a third moiety that links them together. Multifunctional agents comprising three or more functional domains that bind to different portions of a target molecule or different molecules (trispecific agents, tetraspecific agents) are also envisioned.

In general, any suitable method for preparing multivalent, e.g., bivalent, trivalent, or tetravalent antibodies or conjugates may be used to generate the multifunctional agents. In some embodiments the agent comprises a polypeptide chain comprising two V_{H} and two V_{L} regions linked in tandem. In some embodiments, the agent is a diabody, triabody, or tetrabody. In some embodiments, the multispecific, e.g., bispecific agent, comprises two or more chemically linked Fabs, scFvs, single domain antibodies. In some embodiments, multifunctional agents may be made using methods that make use of a suitable linker, such as those described herein. In some embodiments a bifunctional linker comprising two reactive functional groups may be used. It will be understood that suitable modifications may be made to the agents in order to attach them to each other or to a linking moiety, such as addition of a moiety comprising a reactive functional group, and the resulting agent may contain linking portion (e.g., comprising one or more covalent bonds) created by the linkage. Such modifications are within the scope of the multifunctional agents.

### VI. Assays

In some embodiments, any of the agents described herein may be tested in one or more suitable assays to quantify (i) its ability to bind to iC3b, C3dg, C3d, CR2, CR3, CR4, or any combination thereof, (ii) its ability to induce internalization of iC3b, C3dg, and/or C3d, and/or (iii) its ability to inhibit deleterious (e.g., pro-inflammatory) macrophage polarization in the presence of iC3b, C3dg and/or C3d or to otherwise modulate immune system function. In some embodiments multiple agents are generated and screened, e.g., to identify those that are particularly effective

In some embodiments the effect of an agent (e.g., an iC3b/C3dg/C3d inhibitor) or combination of agents (e.g., an iC3b/C3dg/C3d inhibitor and a complement inhibitor) on the functional state of immune cells (e.g., macrophages, dendritic cells, neutrophils, helper T cells, cytotoxic T cells, B cells, natural killer (NK) cells, etc.) may be measured. An agent may be characterized by measuring its effect on any one or more biological activities of immune cells. In some embodiments an effect may involve direct interaction of the agent with the cell of interest. In some embodiments an agent or combination of agents has a direct effect on a first cell type, which in turn exerts an effect on a second cell type, e.g., by cell-cell contact or by secreting substance(s), e.g., cytokine(s), that affect the second cell type. Those of ordinary skill in the art are aware of suitable assays that may be used to measure immune cell function. For example, a functional assay, such as a phagocytosis assay, cytokine secretion assay, or proliferation in response to an appropriate stimulus may be used. The cytokine may be interferon gamma, tumor necrosis factor alpha, or interleukin-2. In some embodiments the cell is a macrophage or microglial cell and the functional assay measures phagocytosis. For example, phagocytosis of cells or beads having one or more C3b degradation products on their surface may be measured. In some embodiments the cell is a cytotoxic T cell or an NK cell, and the functional assay measures production or release of cytotoxic substances such as perform, granzyme B, granulysin and/or cytotoxicity towards target cells. Cytotoxicity towards target cells may be assessed using, e.g., chromium release assays or other assays of membrane integrity such as MTT assay, induction of caspase activity, etc. In some embodiments the cell is a helper T cell, and function may be assayed by cytokine production, ability to promote the differentiation or function of other immune cell subsets, etc. In the case of a Treg, functional state may be assessed by ability of the cells to produce and/or secrete immunosuppressive cytokines or inhibit effector T cells in a functional assay. In some embodiments the functional state may be inferred from gene expression profile, cell surface marker expression, or other phenotypic characteristics. Those of ordinary skill in the art will be aware of appropriate methods of measuring synthesis and/or release of cytokines or cytotoxic substances, measuring gene expression profile, measuring cell surface marker expression, and performing functional assays. For example, synthesis or release of cytokines or cytotoxic substances may be detected using reporter assays (for measuring transcription driven by regulatory regions such as a promoter or enhancer of a gene that encodes a cytokine or cytotoxic protein), ELISA assays, and the like.

In some embodiments the ability of an agent (e.g., an iC3b/C3dg/C3d inhibitor) or combination of agents (e.g., an iC3b/C3dg/C3d inhibitor and a complement inhibitor) to modulate (e.g., to inhibit) T cell activation may be assessed by measuring one or more biological activities that occur during or as a result of T cell activation, such as T cell proliferation or cytokine production. In some embodiments the ability of an agent to modulate (e.g., to inhibit) T-cell responses and cytokine production may be assessed using in vitro assays such as the mixed lymphocyte reaction (measuring proliferation of lymphocytes in an in vitro culture challenged with MHC-incompatible cells), superantigen or cytomegalovirus stimulation assays, and others known in the art. In some embodiments an assay may be performed in vitro using isolated cells (e.g., primary cells or cells from a cell line) that are contacted with an appropriate stimulus (which may include co-stimulation), in vitro. In some embodiments an appropriate stimulus for measuring T cell activation or the effect of an agent on T cell activation may, for example, comprise anti-CD3 and anti-CD28 antibodies. In some embodiments an appropriate stimulus may comprise a cognate antigen presented by MHC (e.g., on an APC or target cell). In some embodiments the effect of one or more agents may be measured by contacting the cells with the agent(s) and the stimulus and measuring one or more biological of the cell (e.g., cytokine production or release, production or release of cytotoxic substances, killing of target cells, etc.).

In some embodiments, immune system cells may be isolated from a subject and their functional state may be measured ex vivo. Cells may, for example, comprise peripheral blood cells (e.g., peripheral blood mononuclear cells) or macrophages from a healthy subject or from a subject suffering from a chronic inflammatory or autoimmune disorder. Immune system cells of different types or subsets, different differentiation states, and/or different functional states may be isolated and/or quantified based, e.g., on cell surface marker expression, using methods such as flow cytometry or fluorescence activated cell sorting, as known in the art. Suitable cell surface markers are known in the art, and antibodies that may be used to stain cells that display such markers are available. Antigen-specific cells specific for a particular antigen may be isolated using MHC tetramers comprising appropriate antigen. Methods of culturing immune system cells and methods of expanding them ex vivo are known in the art. In some embodiments the subject is a human subject.

In some embodiments the ability of an agent (e.g., an iC3b/C3dg/C3d inhibitor) or combination of agents (e.g., an iC3b/C3dg/C3d inhibitor and a complement inhibitor) to modulate, e.g., to inhibit, an immune response may be assessed in a suitable animal model for, e.g., an inflammatory or autoimmune disorder. The assay may measure the ability of the animal to control or eliminate the cancer or infection. In some embodiments immune system cells are isolated from the non-human animal and any of the above-mentioned assays are performed. Those of ordinary skill in the art are aware of numerous non-human animal model systems.

### VI. Pharmaceutical Compositions, Kits, Doses, and Administration

Suitable preparations, e.g., substantially pure preparations of an iC3b/C3dg/C3d inhibitor, complement inhibitor, or both, may be combined with pharmaceutically acceptable carriers or vehicles, etc., to produce an appropriate pharmaceutical composition. The term “pharmaceutically acceptable carrier or vehicle” refers to a non-toxic carrier or vehicle that does not destroy the pharmacological activity of the compound with which it is formulated. One of skill in the art will understand that a carrier or vehicle is “non-toxic” if it is compatible with administration to a subject in an amount appropriate to deliver the compound without causing undue toxicity. Pharmaceutically acceptable carriers or vehicles that may be used include, but are not limited to, water, physiological saline, Ringer's solution, sodium acetate or potassium acetate solution, 5% dextrose, and the like. The composition may include other components as appropriate for the formulation desired, e.g., as discussed herein. Supplementary active compounds, e.g., compounds independently useful for treating a subject suffering from a chronic inflammatory or autoimmune disorder can also be incorporated into the compositions. The iC3b/C3dg/C3d inhibitor may be any iC3b/C3dg/C3d inhibitor, e.g., any of the iC3b/C3dg/C3d inhibitors described herein, in various embodiments. The complement inhibitor may be any complement inhibitor, e.g., any of the complement inhibitors described herein, in various embodiments. All genera, species, and combinations of genera and species of iC3b/C3dg/C3d inhibitor and complement inhibitor are expressly encompassed and disclosed. Where reference is made herein to a government agency responsible for regulating pharmaceutical agents, the government agency may be, e.g., the FDA, EMA, or any other government agency having similar responsibilities, e.g., in a particular jurisdiction.

In some aspects, described herein is a pharmaceutically acceptable iC3b/C3dg/C3d inhibitor or pharmaceutically acceptable composition comprising a iC3b/C3dg/C3d inhibitor, packaged together with a package insert (label) approved by a government agency responsible for regulating pharmaceutical agents, e.g., the FDA or EMA, wherein the package insert states that the iC3b/C3dg/C3d inhibitor or composition comprising an iC3b/C3dg/C3d inhibitor is approved for treatment of a chronic inflammatory or autoimmune disorder. In embodiments the label includes use of the iC3b/C3dg/C3d in combination with a complement inhibitor for treatment of a chronic inflammatory or autoimmune disorder. In some embodiments, the package insert states particular patient and/or disease characteristics or criteria that define a patient population or disease category for treatment of which the Ic3b/C3dg/C3d inhibitor or composition has been approved for use. In some embodiments the disease is age-related macular degeneration. In some embodiments the disorder is an advanced form of AMD, e.g., geographic atrophy. In some embodiments the label includes use of the agent to treat an eye with AMD that has not yet progressed to an advanced form. In some embodiments the subject's other eye may have advanced AMD (GA or neovascular AMD).

In some aspects, described herein is a pharmaceutical pack or kit comprising an iC3b/C3dg/C3d inhibitor and a complement inhibitor, or a pharmaceutical composition comprising anC3b/C3dg/C3d inhibitor and a complement inhibitor. The relative amount of each agent may be selected as appropriate for treatment of the disorder. The pack or kit may contain a package insert or label with directions to treat or describing how to treat a subject who has a disorder of interest using a combination of the C3b/C3dg/C3d inhibitor and a complement inhibitor.

In general, a therapeutic agent, e.g., an C3b/C3dg/C3d inhibitor, a complement inhibitor, or other therapeutic agent, or pharmaceutical composition comprising a therapeutic agent, may be administered using any suitable route of administration. For example, the agent or composition may be administered intravenously, intramuscularly, subcutaneously, intraarterially, by the respiratory route, intraperitoneally, topically, etc. In some embodiments of any of the methods, an C3b/C3dg/C3d inhibitor, a complement inhibitor, or both, is/are administered intravenously. In some embodiments of any of the methods, an C3b/C3dg/C3d inhibitor, a complement inhibitor, or both or both, is/are administered subcutaneously. In some embodiments of any of the methods, an C3b/C3dg/C3d inhibitor is administered intravenously, and a complement inhibitor is administered subcutaneously, or vice versa.

In some embodiments, local administration to the eye may be used. Methods of local administration to the eye include, e.g., intraocular administration, e.g., intraocular injection, e.g., intravitreal injection, choroidal injection, transscleral injection, eyedrops or eye ointments, transretinal, subconjunctival bulbar, intravitreal injection, suprachoroidal injection, subtenon injection, scleral pocket or scleral cutdown injection.

In some embodiments local administration comprises intrathecal or intraventricular administration to the central nervous system, e.g., for treatment of a disorder affecting the central nervous system.

It will be understood that “treatment” or “administration” encompasses, in various embodiments, directly administering a compound or composition to a subject, instructing a third party to administer a compound or composition to a subject, prescribing or suggesting a compound or composition to a subject (e.g., for self-administration), self-administration, and, as appropriate, other means of making a compound or composition available to a subject.

Pharmaceutical compositions suitable for injectable use (e.g., intravenous administration, subcutaneous or intramuscular administration) typically include sterile aqueous solutions (where water soluble) or dispersions and sterile powders for the extemporaneous preparation of sterile injectable solutions or dispersion. Injection encompasses bolus injection, intermittent or continuous infusion, e.g., using an infusion pump, etc. Sterile solutions can be prepared by incorporating the compound in the required amount in an appropriate solvent, optionally with one or a combination of ingredients such as buffers such as acetates, citrates, lactates or phosphates; agents for the adjustment of tonicity such as sodium chloride or dextrose; antibacterial agents such as benzyl alcohol or methyl parabens; antioxidants such as ascorbic acid, glutathione, or sodium bisulfite; chelating agents such as ethylenediaminetetraacetic acid; and other suitable ingredients etc., as desired, followed by filter-based sterilization. One of ordinary skill in the art will be aware of numerous physiologically acceptable compounds that may be included in a pharmaceutical composition. Other useful compounds include, for example, carbohydrates, such as glucose, sucrose, lactose; dextrans; amino acids such as glycine; polyols such as mannitol. These compounds may, for example, serve as bulking agents and/or stabilizers, e.g., in a powder and/or when part of the manufacture or storage process involves lyophilization. Surfactant(s) such as Tween-80, Pluronic-F108/F68, deoxycholic acid, phosphatidylcholine, etc., may be included in a composition, e.g., to increase solubility or to provide microemulsion to deliver hydrophobic drugs. pH can be adjusted with acids or bases, such as hydrochloric acid or sodium hydroxide, if desired. The parenteral preparation can be enclosed in ampoules, disposable syringes or infusion bags or multiple dose vials made of glass or plastic. Preferably solutions for injection are sterile and acceptably free of endotoxin.

Generally, dispersions are prepared by incorporating the active compound into a sterile vehicle which contains a basic dispersion medium and appropriate other ingredients from those enumerated above. In the case of sterile powders for the preparation of sterile injectable solutions, methods of preparation can include vacuum drying and freeze-drying which yields a powder of the active ingredient plus any additional desired ingredient, e.g., from a previously sterile-filtered solution thereof.

For administration by the respiratory route (inhalation), an agent may be delivered in the form of an aerosol spray from a pressured container or dispenser which contains a suitable propellant. A metered dose inhaler (MDI), dry powder inhaler, or nebulizer may be used. The aerosol may comprise liquid and/or dry particles (e.g., dry powders, large porous particles, etc.). Suitable aqueous vehicles useful in various embodiments include water or saline, optionally including an alcohol. In some embodiments the composition comprises a surfactant suitable for introduction into the lung. Other excipients suitable for pulmonary administration can be used. Respiratory administration may be used, e.g., in treating lung cancer, e.g., primary lung cancer or metastasis to the lung from a primary cancer elsewhere, or an infection in the lung.

A variety of different devices are available for respiratory administration. Nebulizers are devices that transform solutions or suspensions of medications into aerosols that are suitable for deposition in the lower airway. Nebulizer types include jet nebulizers, ultrasonic wave nebulizers, and vibrating mesh nebulizers. A partial list of available vibrating mesh nebulizers includes eFlow (Pari), i-Neb (Respironics), MicroAir (Omron), IH50 Nebulizer (Beurer), and Aeroneb® (Aerogen). A Respimat® Soft Mist™ Inhaler (Boeringer Ingelheim) may be used. A metered dose inhaler (MDI) is a handheld aerosol device that uses a propellant to deliver the therapeutic agent. MDIs include a pressurized metal canister that contains pharmacological agent in suspension or solution, propellant, surfactant (typically), and metering valve. Chloroflourocarbons (CFCs) had been widely used as propellants but have been largely replaced by hydrofluorocarbons (HFCs, also known as hydrofluoroalkanes (HFA)) such as HFC-134a and HFC-227ea. Carbon dioxide and nitrogen are other alternatives. A dry powder inhaler (DPI) is a breath-actuated device that delivers the drug in the form of particles contained in a capsule or blister that is punctured prior to use and typically does not employ a propellant. Examples of DPIs currently used to deliver medications for treating asthma and/or COPD include, e.g., Diskus, Aerolizer, HandiHaler, Twisthaler, Flexhaler. Such devices may be used to deliver an C3b/C3dg/C3d inhibitor, complement inhibitor, or both, in various embodiments. Other exemplary DPI devices that may be used in various embodiments include 3M™ Taper and 3M Conix™, TAIFUN® (AKELA Pharma), Acu-Breathe™ (Respirics).

Inhalation accessory devices (IADs) generally fall into 2 categories: spacers and holding chambers. Spacers and holding chambers extend the mouthpiece of the inhaler and direct the mist of medication toward the mouth, reducing medication lost into the air. Using a spacer with an MDI can help reduce the amount of drug that sticks to the back of the throat, improving direction and deposition of medication delivered by MDIs. Valved holding chambers (VHCs) allow for a fine cloud of medication to stay in the spacer until the patient breathes it in through a one-way valve, drawing the dose of medicine into the lungs. Examples include Aerochamber and Optichamber.

Particulate compositions may be characterized on the basis of various parameters such as the fine particle fraction (FPF), the emitted dose, the average particle density, and the mass median aerodynamic diameter (MMAD). Suitable methods are known in the art, some of which are described in U.S. Pat. Nos. 6,942,868 and 7,048,908 and U.S. Publication Nos. 20020146373, 20030012742, and 20040092470. In certain embodiments aerosol particles are between approximately 0.5 μm-10 μm (MMAD), e.g., about 5 μm for respiratory delivery, though smaller or larger particles could also be used. In certain embodiments particles having a mass mean aerodynamic diameter of between 1 μm and 25 μm, e.g., between 1 μm and 10 μm, are used.

A dry particle composition containing particles smaller than about 1 mm in diameter is also referred to herein as a dry powder. A “dry” composition has a relatively low liquid content, so that the particles are readily dispersible, e.g., in a dry powder inhalation device to form an aerosol or spray. A “powder” consists largely or essentially entirely of finely dispersed solid particles that are relatively free flowing and capable of being readily dispersed in an inhalation device and subsequently inhaled by a subject, preferably so that a significant fraction of the particles can reach a desired portion of the respiratory tract. In certain embodiments large porous particles having mean geometric diameters ranging between 3 and 15 μm and tap density between 0.04 and 0.6 g/cm³ are used. See, e.g., U.S. Pat. No. 7,048,908; Edwards, D. et al, Science 276:1868-1871, 1997; and Vanbever, R., et al., Pharmaceutical Res. 16:1735-1742, 1999).

Various considerations for respiratory delivery that may be useful in certain embodiments are discussed in Bisgaard, H., et al., (eds.), Drug Delivery to the Lung, Vol. 26 in “Lung Biology in Health and Disease”, Marcel Dekker, New York, 2002. Aerosol devices are discussed, e.g., in Dolovich M B, Dhand R. Lancet. (2011) 377(9770):1032-45.

Oral administration may be used in certain embodiments. Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. A liquid composition can also be administered orally. Formulations for oral delivery may incorporate agents to improve stability within the gastrointestinal tract and/or to enhance absorption.

For topical application, an agent may be formulated in a suitable ointment containing the active component suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated as a suitable lotion or cream containing a compstatin analog suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

Systemic administration can also be by transmucosal or transdermal means. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration can be accomplished, e.g., through the use of nasal sprays or suppositories. In some embodiments, intranasal administration is used, e.g., to administer a complement inhibitor to a subject in need of treatment for nasal polyposis, chronic rhinosinusitis, or allergic rhinitis. For transdermal administration, the active compounds are typically formulated into ointments, salves, gels, or creams as generally known in the art.

The compounds can also be prepared in the form of suppositories (e.g., with conventional suppository bases such as cocoa butter and other glycerides) or retention enemas for rectal delivery.

In certain embodiments an active agent is prepared with carrier(s) that will protect the compound against rapid elimination from the body, such as a controlled release formulation, including implants and microencapsulated delivery systems. For example, a compound may be incorporated into or encapsulated in a microparticle or nanoparticle formulation. Biodegradable, biocompatible polymers can be used, such as ethylene vinyl acetate, polyanhydrides, polyglycolic acid, collagen, polyorthoesters, polyethers, polylactic acid, PLGA, etc. Liposomes or other lipid-based particles can be used as pharmaceutically acceptable carriers. These can be prepared according to methods known to those skilled in the art, for example, as described in U.S. Pat. No. 4,522,811 and/or other references listed herein. Depot formulations may be used, wherein an active agent released from the depot over time. One of ordinary skill in the art will appreciate that the materials and methods selected for preparation of a controlled release formulation, implant, etc., should be such as to retain activity of the compound. In some embodiments, a composition is free or essentially free of one or more carrier(s) whose primary or only intended purpose or effect would be to result in sustained or controlled release of an active agent, e.g., a complement inhibitor.

When two or more agents (e.g., compounds or compositions) are used or administered “in combination” with each other, also referred to as “combination therapy”, “co-administration”, they may be given at the same time, within overlapping time periods, or sequentially (e.g., separated by up to 2-4 weeks, 4-6 weeks, 6-8 weeks, or 8-12 weeks, in time), at least once, in various embodiments. The agents may be administered in the same composition or can be administered separately but sufficiently closely in time so as to provide the desired therapeutic effect. A person of ordinary skill in the art would readily determine appropriate timing, sequence, and dosages of administration for particular agents and compositions described herein. It will be understood that any sequence may be applied repeatedly, and that different time intervals may be used over a course of treatment. There may be one or more cycles of administration of a first agent, followed by one or more cycles of administration of a second agent, and such cycles may be repeated one or more times. Agents administered in combination may be administered via the same route or different routes in various embodiments. They may be administered in either order in various embodiments. In some embodiments an agent is administered at least once between two doses of another agent. In some embodiments an agent is administered at least once between every second, third, or fourth dose of another agent. In some embodiments, agents are administered within 4, 8, 12, 24, 48, 72, or 96 hours of each other at least once. In some embodiments, agents are administered within 4, 8, 12, 24, 48, 72, or 96 hours of each other multiple times. In some embodiments, a first agent is administered prior to or after administration of the second agent, e.g., sufficiently close in time that the two agents are present at useful levels within the body at least once. In some embodiments, the agents are administered sufficiently close together in time such that no more than 50%, 75%, or 90% of the earlier administered agent has been metabolized to inactive metabolites or eliminated, e.g., excreted, from the body, at the time the second agent is administered.

It will be appreciated that a compound may be provided as a pharmaceutically acceptable salt. Pharmaceutically acceptable salts include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Also, pharmaceutically-acceptable salts can be prepared as alkaline metal or alkaline earth salts, such as sodium, potassium or calcium salts, if appropriate depending on the identity of the active agent.

It will be understood that the pharmaceutically acceptable carriers, compounds, and preparation methods mentioned herein are exemplary and non-limiting. See, e.g., Remington: The Science and Practice of Pharmacy. 21st Edition. Philadelphia, Pa. Lippincott Williams & Wilkins, 2005, for additional discussion of pharmaceutically acceptable compounds and methods of preparing pharmaceutical compositions of various types.

An agent can be used or administered to a subject in an effective amount. In some embodiments, an “effective amount” of an active agent, e.g., an C3b/C3dg/C3d inhibitor or a complement inhibitor refers to an amount of the active agent sufficient to elicit one or more biological effect(s) of interest in, for example, a subject to whom the active agent (or composition) is administered. As will be appreciated by those of ordinary skill in the art, the absolute amount of a particular agent that is effective may vary depending on such factors as the biological endpoint, the particular active agent, the target tissue, etc. Those of ordinary skill in the art will further understand that an “effective amount” may be administered in a single dose, or may be achieved by administration of multiple doses. In some embodiments an effective amount of an agent or composition may be an amount sufficient to achieve one or more of the following: an improvement in one or more symptoms or signs, a decrease in the rate of progression of one or more symptoms or signs (e.g., a decrease in the rate of GA lesion growth). Those of ordinary skill in the art will appreciate that a wide variety of assays are available for detecting and/or quantifying symptoms and signs. An effective amount of a composition, e.g., a pharmaceutical composition, contains an effective amount of one or more agents. If the composition comprises multiple active agents, the agents may be present in an amount such that the overall composition is effective.

In general, appropriate doses depend at least in part upon the potency of the agent, route of administration, etc. In general, dose ranges that are effective and well tolerated can be selected by one of ordinary skill in the art. Such doses can be determined using clinical trials as known in the art. In some embodiments, an animal model is used, for example, to help guide selection of a dose, dose range, or formulation for testing in humans, to assess one or more biological effect(s), etc. Those of ordinary skill in the art will understand that an effective amount for treating a disorder may be established based on the effect of the agent in a population of subjects to whom it is administered, e.g., in a clinical trial, and that the agent may not produce the desired therapeutic effect in every subject. Those of ordinary skill in the art will also understand that certain agents are typically used in combination with other therapies, and that an “effective amount” of such an agent for treating a disorder may be an amount such that the therapeutic effect of interest is produced by the combination of the agent and the other therapies, also used at their effective amounts. Optionally, a dose may be be tailored to the particular recipient, for example, through administration of increasing doses until a preselected desired response is achieved, such as a preselected desired degree of complement inhibition in the case of a complement inhibitor. If desired, the specific dose level for any particular subject may be selected based at least in part upon a variety of factors such as the activity of the specific compound employed, the particular condition being treated and/or its severity, the age, body weight, general health, route of administration, any concurrent medication. In some embodiments an effective amount or dose ranges from about 0.001 to 500 mg/kg body weight, e.g., about 0.01 to 100 mg/kg body weight, e.g., about 0.1 to 50 mg/kg body about 0.1 to 20 mg/kg body weight, e.g., about 1 to 10 mg/kg.

In some embodiments, a method comprises administering at least 5, 10, 15, 20, or 25 doses of an iC3b/C3dg/C3b inhibitor to a subject. In some embodiments, treatment with an iC3b/C3dg/C3b inhibitor is continued over a period at least 3, 6, 9, 12 months, or more, e.g., 1-2 years, 2-5 years, or more. In some embodiments, treatment with an iC3b/C3dg/C3b inhibitor is continued until a chronic inflammatory disorder or autoimmune disorder goes into remission. In some embodiments, between 1 and 5 doses or between 5 and 10 doses of an iC3b/C3dg/C3b inhibitor may be sufficient to cause a chronic inflammatory disorder or autoimmune disorder to go into remission. The subject may be retreated with an iC3b/C3dg/C3b inhibitor if a relapse occurs. In some embodiments the subject is also treated with a complement inhibitor during at least part of the time period during which the subject is being treated with an iC3b/C3dg/C3b inhibitor. The agents may be used in combination for any period of time over which the subject is treated with the iC3b/C3dg/C3b inhibitor. In some embodiments treatment with the complement inhibitor is continued after treatment with an iC3b/C3dg/C3b inhibitor stops.

It will be appreciated that the dosing interval and dosage amount may vary over the course of therapy. For example, in some embodiments, a course of treatment comprises two or more phases, e.g., an induction phase and a subsequent phase. In some embodiments, the induction phase (if used) occurs when a subject initiates therapy with a particular agent or combination of agents. The induction phase can consist of a period of time during which an agent, e.g., an iC3b/C3dg/C3b inhibitor, a complement inhibitor, or both, is/are administered at a higher dose and/or at more frequent intervals and/or using a different route of administration than during the subsequent phase. In some embodiments an induction phase may last for up to 1, 2, 3, 4, 5, 6, 7, 8, 10, or 12 weeks. In some embodiments an induction phase consists of a single loading dose, which is higher than doses administered during the maintenance phase. In some embodiments a dose or dosing interval is adjusted during an induction phase. For example, in some embodiments the dosing interval may be increased over time and/or the dose may be decreased or increased over time during the induction phase. In some embodiments an escalating dose regimen may be used. In some embodiments retreatment may occur on a fixed time schedule.

Further provided herein are methods comprising instructing a patient or health care provider in the use of a composition or method described herein, where the term “instructing” in this context means providing directions for the relevant treatment, treatment regimen, complement system biomarker assay, or the like, by any means, e.g., writing. Instructing can be in the form of prescribing a course of treatment, or can be in the form of package inserts or other written material. It will be understood that such methods may be applied in the context of any composition or method described herein. Instructions may specify one or more acceptable doses, dosing intervals, routes of administration, methods of monitoring the patient, etc.

Further provided are methods comprising marketing a composition or method described herein, wherein “marketing” refers to the promotion (e.g., advertising), selling, distribution of a product (e.g., a pharmaceutical agent or composition) or other activity involving or directed to commercializing a product. It will be understood that such methods may be applied in the context of any composition or method described herein.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. It will be appreciated that the invention is in no way dependent upon particular results achieved in any specific example or with any specific embodiment. Articles such as “a”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. For example, and without limitation, it is understood that where claims or description indicate that a residue at a particular position may be selected from a particular group of amino acids or amino acid analogs, the invention includes individual embodiments in which the residue at that position is any of the listed amino acids or amino acid analogs. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims or from the description above is introduced into another claim. For example, any claim that is dependent on another claim can be modified to include one or more elements, limitations, clauses, or descriptive terms, found in any other claim that is dependent on the same base claim. Furthermore, where the claims recite a composition, it is to be understood that methods of administering the composition according to any of the methods disclosed herein, and methods of using the composition for any of the purposes disclosed herein are included within the scope of the invention, and methods of making the composition according to any of the methods of making disclosed herein are included within the scope of the invention, unless otherwise indicated or unless it would be evident to one of ordinary skill in the art that a contradiction or inconsistency would arise. Methods of treating a subject can include a step of providing a subject in need of such treatment (e.g., a subject who has been diagnosed with the disease or is at increased risk of developing a disease), a step of diagnosing a subject as having a disease and/or a step of selecting a subject for treatment with one or more agents described herein.

Where elements are presented as lists, it is to be understood that each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects or embodiments of the invention, is/are referred to as comprising particular elements, features, etc., certain embodiments of the invention or aspects of the invention consist, or consist essentially of, such elements, features, etc.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Any embodiment, aspect, element, feature, etc., of the present invention may be explicitly excluded from the claims. For example, any agent, formulation, disorder, subject population or characteristic(s), dosing interval, administration route, biomarker, assay, or combination of any of the foregoing, can be explicitly excluded.

We claim:

1. A method of treating a subject who has a chronic inflammatory disorder or autoimmune disorder, the method comprising administering an iC3b/C3dg/C3d inhibitor to the subject.

2. A method of treating a subject who has a chronic inflammatory disorder or autoimmune disorder comprising administering to the subject one or both of:
(a) an iC3b/C3dg/C3d inhibitor and
(b) a complement inhibitor
so that the subject is exposed to both the iC3b/C3dg/C3d inhibitor and the complement inhibitor.

3. The method of claim 1 or claim 2, wherein the subject has a disorder associated with aberrantly high level of C3 fragment accumulation on self cells.

4. The method of any of claims 1-3, wherein the subject has a disorder characterized by non-MAC-mediated cell death.

5. The method of any of claims 1-4, wherein the iC3b/C3dg/C3d inhibitor inhibits pathologic macrophage/microglial polarization.

6. The method of any of claims 1-5, wherein the subject has age-related macular degeneration (AMD).

7. The method of any of claims 1-6, wherein the subject has geographic atrophy.

8. The method of any of claims 1-6, wherein the subject has early or intermediate AMD.

9. The method of any of claims 1-8, wherein the subject has an eye disorder and the iC3b/C3dg/C3d inhibitor is administered by intravitreal injection.

10. The method of any of claims 1-5, wherein the subject has a complement-mediated nephropathy.

11. The method of any of claims 1-5, wherein the subject has a complement-mediated neuromuscular disorder.

12. The method of any of claims 1-11, wherein the subject is under treatment with a complement inhibitor.

13. The method of any of claims 1-11, wherein the method comprises administering a complement inhibitor to the subject.

14. A composition comprising an iC3b/C3dg/C3d inhibitor and a complement inhibitor, optionally wherein the iC3b/C3dg/C3d inhibitor and the complement inhibitor are not linked.

15. The method or composition of any of claims 1-14, wherein the iC3b/C3dg/C3d inhibitor comprises an antibody, aptamer, peptide, polypeptide, or small molecule that binds to one or more of iC3b, C3dg, and C3d.

16. The method or composition of any of claims 1-15, wherein the iC3b/C3dg/C3d inhibitor binds to iC3b, C3dg, or both.

17. The method or composition of any of claims 1-16, wherein the iC3b/C3dg/C3d inhibitor binds to iC3b, C3dg, and C3d.

18. The method or composition of any of claims 1-17, wherein the iC3b/C3dg/C3d inhibitor does not detectably bind to C3.

19. The method or composition of any of claims 1-18, wherein the iC3b/C3dg/C3d inhibitor does not bind to C2.

20. The method or composition of any of claims 1-19, wherein the iC3b/C3dg/C3d inhibitor binds to a neoepitope in iC3b that is retained in C3dg.

21. The method or composition of any of claims 1-20, wherein the iC3b/C3dg/C3d inhibitor comprises an antibody, nucleic acid, peptide, polypeptide, or small molecule that binds to one or more of CR2, CR3, and CR4 but does not mimic the effect of iC3b, C3dg, or C3d.

22. The method or composition of any of claims 1-21, wherein the iC3b/C3dg/C3d inhibitor specifically binds to CR3.

23. The method or composition of any of claims 1-22, wherein the iC3b/C3dg/C3d inhibitor specifically binds to CR3 and CR4.

24. The method or composition of any of claims 1-23, wherein the iC3b/C3dg/C3d inhibitor enhances internalization of C3 degradation fragments by mammalian cells having such fragments covalently attached to their surface.

25. The method or composition of any of claims 2-24, wherein the method or composition uses or comprises a complement inhibitor that comprises an antibody, nucleic acid, peptide, polypeptide, or small molecule that binds to a complement component.

26. The method or composition of claim 25, wherein the complement inhibitor binds to C3, C5, factor B, factor D, MASP-2, or MASP-3

27. The method or composition of claim 25, wherein the complement inhibitor inhibits cleavage of C3, C5, or factor B.

28. The method or composition of claim 25, wherein the complement inhibitor comprises eculizumab, ALXN-1210, pexelizumab, lampalizumab, or a compstatin analog.

29. The method or composition of claim 25, wherein the complement inhibitor comprises a compstatin analog.

30. The method or composition of claim 25, wherein the complement inhibitor comprises a compstatin analog whose sequence comprises any of SEQ ID NOs: 3-41.

31. The method or composition of claim 25, wherein the complement inhibitor comprises a compstatin analog whose sequence comprises SEQ ID NO: 14, 21, 28, 29, 32, 33, 34, 36, or 41.

32. The method or composition of claim 25, wherein the complement inhibitor comprises a clearance-reducing moiety, optionally wherein the clearance reducing moiety comprises PEG, POZ, or a polypeptide.

33. A method of identifying a candidate agent for treatment of a chronic inflammatory disease or autoimmune disease comprising: (a) contacting a population of mammalian cells having iC3b, C3dg, C3d, or a combination thereof on the cell surface with a test agent; (b) measuring the amount of iC3b, C3dg, and C3d on the surface of the cells; and (c) identifying the test agent as a candidate agent for treatment of a chronic inflammatory disease or autoimmune disease if the amount of iC3b, C3dg, and C3d on the surface of the cells is less than that present on control cells not contacted with the test agent.

34. The method of claim 33, wherein the amount of iC3b, C3dg, and C3d on the surface of the one or more cells is measured using a labeled antibody that binds to C3d.

35. The method of claim 33 or claim 34, wherein the cells are human cells.

36. A method of identifying a candidate agent for treatment of a chronic inflammatory disease or autoimmune disease comprising: (a) contacting a population of mammalian cells having iC3b, C3dg, C3d, or a combination thereof on the cell surface with a population of macrophages or microglia in the presence of a test agent; (b) measuring the extent to which the macrophages or microglia become polarized towards a tissue destructive, pro-inflammatory phenotype; and (c) identifying the test agent as a candidate agent for treatment of a chronic inflammatory disease or autoimmune disease if the extent to which the macrophages or microglia become polarized towards a tissue destructive, pro-inflammatory phenotype is less than the extent to which control macrophages or microglia contacted with mammalian cells having iC3b, C3dg, C3d, or a combination thereof on the cell surface in the absence of the test agent become polarized towards a tissue destructive, pro-inflammatory phenotype.

37. The method of claim 36, wherein the macrophages or microglia and the mammalian cells are human cells.

38. A method of characterizing agent for treatment of a chronic inflammatory disease or autoimmune disease, the method comprising steps of:
(a) determining a cell surface level of one or more of iC3b, C3dg, and C3d on mammalian cells when the agent is present; and
(b) comparing the determined level with that observed under otherwise comparable conditions absent the agent or when a reference agent of known effect on the cell surface level(s) is present.

39. A method of characterizing an agent for treatment of a chronic inflammatory disease or autoimmune disease, the method comprising steps of:
(a) determining extent of macrophage or microglia polarization toward a tissue destructive, pro-inflammatory phenotype when the agent is present; and
(b) comparing the determined extent with that observed under otherwise comparable conditions absent the agent or when a reference agent of known effect on extent of macrophage or microglia polarization toward the tissue destructive, pro-inflammatory phenotype is present.
